(19) United States
(12) Patent Application Publication
(10) Pub. No.: US 2010/0303832 A1
(43) Pub. Date: Dec. 2, 2010

(54) GENES AND POLYMORPHISMS ASSOCIATED WITH AMD

(76) Inventor: Gregory S. Hageman, Salt Lake City, UT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/740,959

(22) PCT Filed: Nov. 3, 2008
(86) PCT No.: PCT/US08/82282

§ 371 Date: Aug. 16, 2010

Related U.S. Application Data
(60) Provisional application No. 60/984,702, filed on Nov. 1, 2007.

(51) Int. Cl. A61K 39/395; C12Q 1/68; G01N 33/50; G01N 33/561; C40B 30/04; A61K 38/02; A61K 31/7088; C07H 21/04; A61P 27/02

(52) U.S. Cl. 424/172.1; 435/6; 435/7.92; 436/516; 436/98; 506/9; 514/20.8; 514/44 R; 514/44 A; 536/24.31

(57) ABSTRACT

The invention relates to genes, gene polymorphisms, and genetic profiles associated with an elevated or a reduced risk of alternative complement cascade deregulation disease such as AMD. The invention provides methods and reagents for determination of risk, diagnosis and treatment of such diseases. In an embodiment, the present invention provides methods and reagents for determining sequence variants in the genome of a patient which facilitate assessment of risk for developing such diseases.

### RELATED APPLICATIONS

This application claims the benefit of the priority date of U.S. Provisional Application No. 60/984,702, which was filed on Nov. 1, 2007, the contents of which are incorporated herein by reference in their entirety.

### FEDERALLY SPONSORED RESEARCH AND DEVELOPMENT

This invention was made with government support under NIH R01 EY11515 and R24 EY017404, awarded by the National Institutes of Health. The government has certain rights in the invention.

### FIELD OF THE INVENTION

The invention relates to risk determination, diagnosis and prognosis of complement-related disorders such as age-related macular degeneration (AMD).

### BACKGROUND OF THE INVENTION

Age-related macular degeneration (AMD) is the leading cause of irreversible vision loss in the developed world, affecting approximately 15% of individuals over the age of 60. The prevalence of AMD increases with age: mild, or early, forms occur in nearly 30%, and advanced forms in about 7%, of the population that is 75 years and older. Clinically, AMD is characterized by a progressive loss of central vision attributable to degenerative changes that occur in the macula, a specialized region of the neural retina and underlying tissues. In the most severe, or exudative, form of the disease neovascular fronds derived from the choroidal vasculature breach Bruch's membrane and the retinal pigment epithelium (RPE) typically leading to detachment and subsequent degeneration of the retina.

Numerous studies have implicated inflammation in the pathobiology of AMD (Anderson et al. (2002) Am. J. Opthalmol. 134:41 1-31; Hageman et al. (2001) Prog. Retin. Eye Res. 20:705-32; Mullins et al. (2000) Faseb J. 14:835-46; Johnson et al. (2001) Exp. Eye Res. 73:887-96; Crabb et al. (2002)PNAS 99:14682-7; Bok (2005) PNAS 102:7053-4). Dysfunction of the complement pathway may induce significant bystander damage to macular cells, leading to atrophy, degeneration, and the elaboration of choroidal neovascular membranes, similar to damage that occurs in other complement-mediated disease processes (Hageman et al. (2005) PNAS 102:7227-32: Morgan and Walport (1991) Immunol. Today 12:301-6; Kinoshita (1991) Immunol. Today 12:291-5; Holers and Thurman (2004) Mol. Immunol. 41: 147-52).

AMD, a late-onset complex disorder, appears to be caused and/or modulated by a combination of genetic and environmental factors. According to the prevailing hypothesis, the majority of AMD cases is not a collection of multiple single-gene disorders, but instead represents a quantitative phenotype, an expression of interaction of multiple susceptibility loci. The number of loci involved, the attributable risk conferred, and the interactions between various loci remain obscure, but significant progress has been made in determining the genetic contribution to these diseases. See, for example, U.S. Patent Publication No. 20070020647, U.S. Patent Publication No. 20060281120, PCT publication WO 2008/013893, and U.S. Patent Publication No. 20080152659.

Thus, variations in complement-related genes have been found to be correlated with AMD. A common haplotype in the complement regulatory gene factor H(HF1/CFH) predisposes individuals to age-related macular degeneration. Hageman et al., 2005, Proc. Nat'l Acad Sci 102: 7227-32. Similarly, the non-synonymous polymorphism at amino acid position 1210 in exon 22 of the Factor H gene is strongly associated with AMD. See, for example, Hageman et al. WO 2006/088950; Hageman et al. WO2007/095287 and Hughes et al., 2006, Nat. Genet. 38:458-62. Deletions and other variations in other genes of the RCA locus (such as CFH-related 3 [FHR3] and CFH-related 1 [FHR1], among others) have also been correlated with AMD. See, for example, International Publication No. WO2008/008986, and Hughes et al., 2006, Nat. Genet. 38:458-62. Sequence variations in other complement regulators, such as complement component C2 and complement factor B, which are closely linked on chromosome 6, have also been associated with AMD risk. See, for example, International Publication No. WO 2007/095185. Closely linked genes on chromosome 10, including LOC387715, HTRA1, and PLEKHA1 have also been shown to harbor sequence variations informative of AMD risk. See, for example, U.S. Patent Application Publication No. US 2006/0281120; International Publication No. WO 2007/044897; and International Publication No. WO 2008/013893

Analysis of single polynucleotide polymorphisms (SNPs) is a powerful technique for diagnosis and/or determination of risk for complement-related disorders such as AMD.

### SUMMARY

The invention arises, in part, from a high density, large sample size, genetic association study designed to detect genetic characteristics associated with complement cascade dysregulation diseases such as AMD. The study revealed a large number of new genes never before reported and a still larger number of SNPs (and/or combination of certain SNPs) which were not previously reported to be associated with risk for, or protection from, AMD. The invention disclosed herein thus relates to the discovery of new genes and polymorphisms that are associated with the development of age-related macular degeneration (AMD). The polymorphisms are found within or near genes such as CCL28, FBN2, ADAM12, PTPRC, IGLC1, HS3ST4, PRELP, PPID, SPOCK, APOB, SLC2A2, COL4A1, COL6A3, MYOC, ADAM19, FGFR2, C8A, FCN1, IFNAR2, C1NH, C7, and ITGA4, which are shown in Tables I and II. The informative value of many of the specific SNPs disclosed herein has never before been recognized or reported, as far as the inventor is aware. The invention provides methods of screening for individuals at risk of developing AMD and/or for predicting the likely progression of early- or mid-stage established disease and/or for predicting the likely outcome of a particular therapeutic or prophylactic strategy.

In one aspect, the invention provides a diagnostic method of determining an individual's propensity to complement dysregulation comprising screening (directly or indirectly) for the presence or absence of a genetic profile characterized by polymorphisms in the individual's genome associated with complement dysregulation, wherein the presence of said genetic profile is indicative of the individual's risk of complement dysregulation. The profile may reveal that the individual's risk is increased, or decreased, as the profile may evidence increased risk for, or increased protection from, developing AMD. A genetic profile associated with complement dysregulation comprises one or more, typically multiple, single nucleotide polymorphisms selected from Table I or Table II. In certain embodiments, a genetic profile associated with complement dysregulation comprises any combination of at least 2, at least 5, or at least 10 single nucleotide polymorphisms selected from Table I or Table II.

In one aspect, the invention provides a diagnostic method of determining an individual's propensity to develop, or for predicting the course of progression, of AMD, comprising screening (directly or indirectly) for the presence or absence of a genetic profile in the any one of the genes, or combinations thereof, listed in Tables I and/or II, which are informative of an individual's (increased or decreased) risk for developing AMD. A genetic profile comprises one or more, typically multiple, single nucleotide polymorphisms selected from at least one gene, typically multiple, shown in Table I or II. In other embodiments, a genetic profile comprises any combination of at least 2, at least 5, or at least 10 single nucleotide polymorphisms selected from at least one gene, typically multiple, shown in Table I or II.

In one embodiment, a method for determining an individual's propensity to develop, or for predicting the course of progression of, age-related macular degeneration, comprises screening for a combination of at least one, typically multiple, risk-associated polymorphism and at least one, typically multiple, protective polymorphism set forth in Table I or II. For example, the method may comprise screening for a SNP selected from the group consisting of: rs7380703, rs10057855, rs1676717, rs1932433, rs1065464, rs4441276, rs947367, rs8396, rs1229729, rs1229731, rs10057405, rs17670373, rs331075, rs12714097, rs7646014, rs7338606, rs3770115, rs1874573, rs2236875, rs12992087, rs1621212, rs7689418, rs4926, rs11842143, rs12035960, rs12189024, rs2961633, rs2981582, MRD_{—}4048, rs17676236, rs6891153, MRD_{—}4044, rs10117466, rs2826552, rs2271708, rs1055021, and rs1055021, or combinations thereof. Risk polymorphisms indicate that an individual has increased susceptibility to developing AMD relative to the control population. Protective polymorphisms indicate that the individual has a reduced likelihood of developing AMD relative to the control population. Neutral polymorphisms do not segregate significantly with risk or protection, and have limited or no diagnostic or prognostic value.

Additional, previously known informative polymorphisms may and typically will be included in the screen. For example, additional risk-associated polymorphisms may include rs1061170, rs203674, rs1061147, rs2274700, rs12097550, rs203674, a polymorphism in exon 22 of CFH(R1210C), rs9427661, rs9427662, rs10490924, rs11200638, rs2230199, rs2511989, rs3753395, rs1410996, rs393955, rs403846, rs1329421, rs10801554, rs12144939, rs12124794, rs2284664, rs16840422, rs6695321, rs2511989, rs1409153, rs10922153, rs12066959, and rs12027476. Additional protection-associated polymorphisms may include: rs800292, rs3766404, rs529825, rs641153, rs4151667, rs547154, and rs9332739. In one embodiment, the screening incorporates one or more polymorphisms from the RCA locus, such as those included in Table VI.

In another embodiment, a method for determining an individual's propensity to develop, or for predicting the course of progression, of AMD, comprises screening additionally for deletions within the RCA locus (i.e., a region of DNA sequence located on chromosome one that extends from the Complement Factor H(CFH) gene through the CD46 gene (also known as the MCP gene, e.g., from CFH through complement factor 13B) that are associated with AMD risk or protection. An exemplary deletion that is protective of AMD is a deletion at least portions of the FHR3 and FHR1 genes. See, e.g., Hageman et al., 2006, “Extended haplotypes in the complement factor H(CFH) and CFH-related (CFHR) family of genes protect against age-related macular degeneration: characterization, ethnic distribution and evolutionary implications,”Ann Med. 38:592-604 and US Patent Publication No. 2008/152659.

The methods may include inspecting a data set indicative of genetic characteristics previously derived from analysis of the individual's genome. A data set of genetic characteristics of the individual may include, for example, a listing of single nucleotide polymorphisms in the patient's genome or a complete or partial sequence of the individual's genomic DNA. Alternatively, the methods include obtaining and analyzing a nucleic acid sample (e.g., DNA or RNA) from an individual to determine whether the DNA contains informative polymorphisms in one or more of the genes shown in Tables I and/or II. In another embodiment, the methods include obtaining a biological sample from the individual and analyzing the sample from the individual to determine whether the individual's proteome contains an allelic variant isoform that is a consequence of the presence of a polymorphisms in the individual's genome.

In another aspect, the invention provides a method of treating, preventing, or delaying development of symptoms of AMD in an individual (e.g., an individual in whom a genetic profile indicative of elevated risk of developing AMD is detected), comprising prophylactically or therapeutically treating an individual identified as having a genetic profile in one or more of the genes, typically multiple, shown in Tables I and/or II, which are indicative of increased risk of development or progression of AMD, wherein the genetic profile includes one or more single nucleotide polymorphisms selected from Tables I and/or II.

In one embodiment, the method of treating or preventing AMD in an individual comprises prophylatically or therapeutically treating the an individual by administering a composition comprising a Factor H polypeptide. The Factor H polypeptide may be a wild type Factor H polypeptide or a variant Factor H polypeptide. The Factor H polypeptide may be a Factor H polypeptide with a sequence encoded by a protective or neutral allele. In one embodiment, the Factor H polypeptide is encoded by a Factor H protective haplotype. A protective Factor H haplotype can encode an isoleucine residue at amino acid position 62 and/or an amino acid other than a histidine at amino acid position 402. For example, a factor H polypeptide can comprise an isoleucine residue at amino acid position 62, a tyrosine residue at amino acid position 402, and/or an arginine residue at amino acid position 1210. Exemplary Factor H protective haplotypes include the H2 haplotype or the H4 haplotype. Alternatively, the Factor H polypeptide may be encoded by a Factor H neutral haplotype. A neutral haplotype encodes an amino acid other than an isoleucine at amino acid position 62 and an amino acid other than a histidine at amino acid position 402. Exemplary Factor H neutral haplotypes include the H3 haplotype or the H5 haplotype. For details on therapeutic forms of CFH, and how to make and use them, see U.S. Patent Publication No. 20070060247, the disclosure of which is incorporated herein by reference.

In some embodiments, the method of treating or preventing AMD in an individual includes prophylactically or therapeutically treating the individual by inhibiting a variant polypeptide encoded by a gene selected from Table I or II in the individual. A variant polypeptide encoded by a gene selected from Table I or II can be inhibited, for example, by administering an antibody or other protein that binds to the variant polypeptide. Alternatively, the variant polypeptide can be inhibited by administering a nucleic acid inhibiting its expression or activity, such as an inhibitory RNA, a nucleic acid encoding an inhibitory RNA, an antisense nucleic acid, or an aptamer.

In other embodiments, the method of treating or preventing AMD in an individual includes prophylactically or therapeutically treating the individual by inhibiting Factor B and/or C2 in the individual. Factor B can be inhibited, for example, by administering an antibody or other protein (e.g., an antibody variable domain, an addressable fibronectin protein, etc.) that binds Factor B. Alternatively, Factor B can be inhibited by administering a nucleic acid inhibiting Factor B expression or activity, such as an inhibitory RNA, a nucleic acid encoding an inhibitory RNA, an antisense nucleic acid, or an aptamer, or by administering a small molecule that interferes with Factor B activity (e.g., an inhibitor of the protease activity of Factor B). C2 can be inhibited, for example, by administering an antibody or other protein (e.g., an antibody variable domain, an addressable fibronectin protein, etc.) that binds C2. Alternatively, C2 can be inhibited by administering a nucleic acid inhibiting C2 expression or activity, such as an inhibitory RNA, a nucleic acid encoding an inhibitory RNA, an antisense nucleic acid, or an aptamer, or by administering a small molecule that interferes with C2 activity (e.g., an inhibitor of the protease activity of C2).

In yet other embodiments, the method of treating or preventing AMD in an individual includes prophylactically or therapeutically treating the individual by inhibiting HTRA1 in the individual. HTRA1 can be inhibited, for example, by administering an antibody or other protein (e.g. an antibody variable domain, an addressable fibronectin protein, etc.) that binds HTRA1. Alternatively, HTRA1 can be inhibited by administering a nucleic acid inhibiting HTRA1 expression or activity, such as an inhibitory RNA, a nucleic acid encoding an inhibitory RNA, an antisense nucleic acid, or an aptamer, or by administering a small molecule that interferes with HTRA1 activity (e.g. an inhibitor of the protease activity of HTRA1).

In another aspect, the invention provides detectably labeled oligonucleotide probes or primers for hybridization with DNA sequence in the vicinity of at least one polymorphism to facilitate identification of the base present in the individual's genome. In one embodiment, a set of oligonucleotide primers hybridizes to a region adjacent to at least one polymorphism in one of the gene shown in Table I or II for inducing amplification thereof, thereby facilitating sequencing of the region and determination of the base present in the individual's genome at the sites of the polymorphism. Preferred polymorphisms for detection include the polymorphisms listed in Tables I and II. Further, one of skill in the art will appreciate that other methods for detecting polymorphisms are well known in the art.

In another aspect, the invention relates to a healthcare method that includes authorizing the administration of, or authorizing payment for the administration of, a diagnostic assay to determine an individual's susceptibility for development or progression of AMD comprising screening for the presence or absence of a genetic profile in at least one gene, typically multiple, shown in Table I or II, wherein the genetic profile comprises one or more SNPs selected from at least one gene, typically multiple, shown in Table I or II.

### DETAILED DESCRIPTION OF THE INVENTION

### I. Definitions and Conventions

The term “polymorphism” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and may be homozygous or heterozygous for allelic forms. The first identified allelic form is arbitrarily designated the reference form or allele; other allelic forms are designated as alternative or variant alleles. The most frequently occurring allelic form in a selected population is typically referred to as the wild-type form.

A “polymorphic site” is the position or locus at which sequence divergence occurs at the nucleic acid level and is sometimes reflected at the amino acid level. The polymorphic region or polymorphic site refers to a region of the nucleic acid where the nucleotide difference that distinguishes the variants occurs, or, for amino acid sequences, a region of the amino acid sequence where the amino acid difference that distinguishes the protein variants occurs. A polymorphic site can be as small as one base pair, often termed a “single nucleotide polymorphism” (SNP). The SNPs can be any SNPs in loci identified herein, including intragenic SNPs in exons, introns, or upstream or downstream regions of a gene, as well as SNPs that are located outside gene sequences. Examples of such SNPs include, but are not limited to, those provided in the tables herein below.

Individual amino acids in a sequence are represented herein as AN or NA, wherein A is the amino acid in the sequence and N is the position in the sequence. In the case that position N is polymorphic, it is convenient to designate the more frequent variant as A₁N and the less frequent variant as NA₂. Alternatively, the polymorphic site, N, is represented as A₁NA₂, wherein A₁ is the amino acid in the more common variant and A₂ is the amino acid in the less common variant. Either the one-letter or three-letter codes are used for designating amino acids (see Lehninger, Biochemistry 2nd ed., 1975, Worth Publishers, Inc. New York, N.Y.: pages 73-75, incorporated herein by reference). For example, 150V represents a single-amino-acid polymorphism at amino acid position 50 of a given protein, wherein isoleucine is present in the more frequent protein variant in the population and valine is present in the less frequent variant.

Similar nomenclature may be used in reference to nucleic acid sequences. In the Tables provided herein, each SNP is depicted by “N₁/N₂” where N₁ is a nucleotide present in a first allele referred to as Allele 1, and N₂ is another nucleotide present in a second allele referred to as Allele 2. It will be clear to those of skill in the art that in a double-stranded form, the complementary strand of each allele will contain the complementary base at the polymorphic position.

The term “genotype” as used herein denotes one or more polymorphisms of interest found in an individual, for example, within a gene of interest. Diploid individuals have a genotype that comprises two different sequences (heterozygous) or one sequence (homozygous) at a polymorphic site.

The term “haplotype” refers to a DNA sequence comprising one or more polymorphisms of interest contained on a subregion of a single chromosome of an individual. A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms in the regulation of complement activation (RCA) locus, which includes gene sequences for complement factor H(CFH), FHR3, FHR1, FHR4, FHR2, FHR5, and F13B and intergenic sequences (i.e., intervening intergenic sequences, upstream sequences, and downstream sequences that are in linkage disequilibrium with polymorphisms in the genic region). The term “haplotype” can refer to a set of single nucleotide polymorphisms (SNPs) found to be statistically associated on a single chromosome. A haplotype can also refer to a combination of polymorphisms (e.g., SNPs) and other genetic markers (e.g., a deletion) found to be statistically associated on a single chromosome. A haplotype, for instance, can also be a set of maternally inherited alleles, or a set of paternally inherited alleles, at any locus.

The term “genetic profile,” as used herein, refers to a collection of one or more single nucleotide polymorphisms comprising polymorphisms shown in Table I and/or II, optionally in combination with other genetic characteristics such as deletions, additions or duplications, and optionally combined with other SNPs known to be associated with AMD risk or protection. Thus, a genetic profile, as the phrase is used herein, is not limited to a set of characteristics defining a haplotype, and may comprise SNPs from diverse regions of the genome. For example, a genetic profile for AMD comprises one or a subset of single nucleotide polymorphisms selected from Table I and/or Table II, optionally in combination with other genetic characteristics known to be associated with AMD. It is understood that while one SNP in a genetic profile may be informative of an individual's increased or decreased risk (i.e., an individual's propensity or susceptibility) to develop a complement-related disease such as AMD, more than one SNP in a genetic profile may and typically will be analyzed and will be more informative of an individual's increased or decreased risk of developing a complement-related disease. A genetic profile may include at least one SNP disclosed herein in combination with other polymorphisms or genetic markers (e.g., a deletion) and/or environmental factors (e.g., smoking or obesity) known to be associated with AMD. In some cases, a SNP may reflect a change in regulatory or protein coding sequences that change gene product levels or activity in a manner that results in increased likelihood of development of a disease. In addition, it will be understood by a person of skill in the art that one or more SNPs that are part of a genetic profile may be in linkage disequilibrium with, and serve as a proxy or surrogate marker for another genetic marker or polymorphism that is causative, protective, or otherwise informative of disease.

The term “gene,” as used herein, refers to a region of a DNA sequence that encodes a polypeptide or protein, intronic sequences, promoter regions, and upstream (i.e., proximal) and downstream (i.e., distal) non-coding transcription control regions (e.g., enhancer and/or repressor regions).

The term “allele,” as used herein, refers to a sequence variant of a genetic sequence (e.g., typically a gene sequence as described hereinabove, optionally a protein coding sequence). For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs. In certain embodiments of the invention, an allele is defined by the genotypes of at least 1, 2, 4, 8 or 16 or more SNPs (including those provided in Tables I and II below) in a gene.

A “causative” SNP is a SNP having an allele that is directly responsible for a difference in risk of development or progression of AMD. Generally, a causative SNP has an allele producing an alteration in gene expression or in the expression, structure, and/or function of a gene product, and therefore is most predictive of a possible clinical phenotype. One such class includes SNPs falling within regions of genes encoding a polypeptide product, i.e. “coding SNPs” (cSNPs). These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic disease. Examples of genes in which a SNP within a coding sequence causes a genetic disease include sickle cell anemia and cystic fibrosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of, or predisposition to, or an increased risk in developing the disease. These SNPs, although not causative, are nonetheless also useful for diagnostics, disease predisposition screening, and other uses.

An “informative” or “risk-informative” SNP refers to any SNP whose sequence in an individual provides information about that individual's relative risk of development or progression of AMD. An informative SNP need not be causative. Indeed, many informative SNPs have no apparent effect on any gene product, but are in linkage disequilibrium with a causative SNP. In such cases, as a general matter, the SNP is increasingly informative when it is more tightly in linkage disequilibrium with a causative SNP. For various informative SNPs, the relative risk of development or progression of AMD is indicated by the presence or absence of a particular allele and/or by the presence or absence of a particular diploid genotype.

The term “linkage” refers to the tendency of genes, alleles, loci, or genetic markers to be inherited together as a result of their location on the same chromosome or as a result of other factors. Linkage can be measured by percent recombination between the two genes, alleles, loci, or genetic markers. Some linked markers may be present within the same gene or gene cluster.

In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium is influenced by a number of factors including genetic linkage, the rate of recombination, the rate of mutation, random drift, non-random mating, and population structure. “Linkage disequilibrium” or “allelic association” thus means the non-random association of a particular allele or genetic marker with another specific allele or genetic marker more frequently than expected by chance for any particular allele frequency in the population. A marker in linkage disequilibrium with an informative marker, such as one of the SNPs listed in Tables I or II can be useful in detecting susceptibility to disease. A SNP that is in linkage disequilibrium with a causative, protective, or otherwise informative SNP or genetic marker is referred to as a “proxy” or “surrogate” SNP. A proxy SNP may be in at least 50%, 60%, or 70% in linkage disequilibrium with the causative SNP, and preferably is at least about 80%, 90%, and most preferably 95%, or about 100% in LD with the genetic marker.

A “nucleic acid,” “polynucleotide,” or “oligonucleotide” is a polymeric form of nucleotides of any length, may be DNA or RNA, and may be single- or double-stranded. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA) and any other polymers capable of serving as a template for an amplification reaction using an amplification technique, for example, a polymerase chain reaction, a ligase chain reaction, or non-enzymatic template-directed replication.

Oligonucleotides are usually prepared by synthetic means. Nucleic acids include segments of DNA, or their complements spanning any one of the polymorphic sites shown in the Tables provided herein. Except where otherwise clear from context, reference to one strand of a nucleic acid also refers to its complement strand. The segments are usually between 5 and 100 contiguous bases, and often range from a lower limit of 5, 10, 12, 15, 20, or 25 nucleotides to an upper limit of 10, 15, 20, 25, 30, 50 or 100 nucleotides (where the upper limit is greater than the lower limit). Nucleic acids between 5-10, 5-20, 10-20, 12-30, 15-30, 10-50, 20-50 or 20-100 bases are common. The polymorphic site can occur within any position of the segment. The segments can be from any of the allelic forms of DNA shown in the Tables provided herein.

“Hybridization probes” are nucleic acids capable of binding in a base-specific manner to a complementary strand of nucleic acid. Such probes include nucleic acids and peptide nucleic acids. Hybridization is usually performed under stringent conditions which are known in the art. A hybridization probe may include a “primer.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The nucleic acids, including any primers, probes and/or oligonucleotides can be synthesized using a variety of techniques currently available, such as by chemical or biochemical synthesis, and by in vitro or in vivo expression from recombinant nucleic acid molecules, e.g., bacterial or retroviral vectors. For example, DNA can be synthesized using conventional nucleotide phosphoramidite chemistry and the instruments available from Applied Biosystems, Inc. (Foster City, Calif.); DuPont (Wilmington, Del.); or Milligen (Bedford, Mass.). When desired, the nucleic acids can be labeled using methodologies well known in the art such as described in U.S. Pat. Nos. 5,464,746; 5,424,414; and 4,948,882 all of which are herein incorporated by reference. In addition, the nucleic acids can comprise uncommon and/or modified nucleotide residues or non-nucleotide residues, such as those known in the art.

An “isolated” nucleic acid molecule, as used herein, is one that is separated from nucleotide sequences which flank the nucleic acid molecule in nature and/or has been completely or partially purified from other biological material (e.g., protein) normally associated with the nucleic acid. For instance, recombinant DNA molecules in heterologous organisms, as well as partially or substantially purified DNA molecules in solution, are “isolated” for present purposes.

The term “target region” refers to a region of a nucleic acid which is to be analyzed and usually includes at least one polymorphic site.

“Stringent” as used herein refers to hybridization and wash conditions at 50° C. or higher. Other stringent hybridization conditions may also be selected. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 50° C. As other factors may significantly affect the stringency of hybridization, including, among others, base composition, length of the nucleic acid strands, the presence of organic solvents, the extent of base mismatching, and the combination of parameters is more important than the absolute measure of any one.

Generally, increased or decreased risk-associated with a polymorphism or genetic profile for a disease is indicated by an increased or decreased frequency, respectively, of the disease in a population or individuals harboring the polymorphism or genetic profile, as compared to otherwise similar individuals, who are for instance matched by age, by population, and/or by presence or absence of other polymorphisms associated with risk for the same or similar diseases. The risk effect of a polymorphism can be of different magnitude in different populations. A polymorphism, haplotype, or genetic profile can be negatively associated (“protective polymorphism”) or positively associated (“predisposing polymorphism”) with a complement-related disease such as AMD. The presence of a predisposing genetic profile in an individual can indicate that the individual has an increased risk for the disease relative to an individual with a different profile. Conversely, the presence of a protective polymorphism or genetic profile in an individual can indicate that the individual has a decreased risk for the disease relative to an individual without the polymorphism or profile.

The terms “susceptibility,” “propensity,” and “risk” refer to either an increased or decreased likelihood of an individual developing a disorder (e.g., a condition, illness, disorder or disease) relative to a control and/or non-diseased population. In one example, the control population may be individuals in the population (e.g., matched by age, gender, race and/or ethnicity) without the disorder, or without the genotype or phenotype assayed for.

The terms “diagnose” and “diagnosis” refer to the ability to determine or identify whether an individual has a particular disorder (e.g., a condition, illness, disorder or disease). The term prognose or prognosis refers to the ability to predict the course of the disease and/or to predict the likely outcome of a particular therapeutic or prophylactic strategy.

The term “screen” or “screening” as used herein has a broad meaning. It includes processes intended for the diagnosis or for determining the susceptibility, propensity, risk, or risk assessment of an asymptomatic subject for developing a disorder later in life. Screening also includes the prognosis of a subject, i.e., when a subject has been diagnosed with a disorder, determining in advance the progress of the disorder as well as the assessment of efficacy of therapy options to treat a disorder. Screening can be done by examining a presenting individual's DNA, RNA, or in some cases, protein, to assess the presence or absence of the various SNPs disclosed herein (and typically other SNPs and genetic or behavioral characteristics) so as to determine where the individual lies on the spectrum of disease risk-neutrality-protection. Proxy SNPs may substitute for any of these SNPs. A sample such as a blood sample may be taken from the individual for purposes of conducting the genetic testing using methods known in the art or yet to be developed. Alternatively, if a health provider has access to a pre-produced data set recording all or part of the individual's genome (e.g., a listing of SNPs in the patient;s genome) screening may be done simply by inspection of the database, optimally by computerized inspection. Screening may further comprise the step of producing a report identifying the individual and the identity of alleles at the site of at least one or more polymorphisms shown in Table I or II.

### II. Introduction

A study was conducted to elucidate potential associations between complement system genes and other selected genes with age-related macular degeneration (AMD). The associations discovered form the basis of the present invention, which provides methods for identifying individuals at increased risk, or at decreased risk, relative to the general population for a complement-related disease such as AMD. The present invention also provides kits, reagents and devices useful for making such determinations. The methods and reagents of the invention are also useful for determining prognosis.

### Use of Polymorphisms to Detect Risk and Protection

The present invention provides a method for detecting an individual's increased or decreased risk for development or progression of a complement-related disease such as AMD by detecting the presence of certain polymorphisms present in the individual's genome that are informative of his or her future disease status (including prognosis and appearance of signs of disease). The presence of such a polymorphism can be regarded as indicative of increased or decreased risk for the disease, especially in individuals who lack other predisposing or protective polymorphisms for the same disease(s). Even in cases where the predictive contribution of a given polymorphism is relatively minor by itself, genotyping contributes information that nevertheless can be useful for a characterization of an individual's predisposition to developing a disease. The information can be particularly useful when combined with genotype information from other loci (e.g., the presence of a certain polymorphism may be more predictive or informative when used in combination with at least one other polymorphism).

### III. New SNPs Associated with Propensity to Develop Disease

In order to identify new single nucleotide polymorphisms (SNPs) associated with increased or decreased risk of developing complement-related diseases such as age-related macular degeneration (AMD), 74 complement pathway-associated genes (and a number of inflammation-associated genes including toll-like receptors, or TLRs) were selected for SNP discovery. New SNPs in the candidate genes were discovered from a pool of 475 DNA samples derived from study participants with a history of AMD using a multiplexed SNP enrichment technology called Mismatch Repair Detection (ParAllele Biosciences/Affymetrix), an approach that enriches for variants from pooled samples. This SNP discovery phase (also referred to herein as Phase I) was conducted using DNA derived solely from individuals with AMD based upon the rationale that the discovered SNPs might be highly relevant to disease (e.g., AMD-associated).

### IV. Association of SNPs and Complement-Related Conditions

In Phase II of the study, 1162 DNA samples were employed for genotyping known and newly discovered SNPs in 340 genes. Genes investigated in Phase II included the complement and inflammation-associated genes used for SNP Discovery (Phase I). The remaining genes were selected based upon a tiered strategy, which was designed as follows. Genes received the highest priority if they fell within an AMD-harboring locus established by genome-wide linkage analysis or conventional linkage, or if they were differentially expressed at the RPE-choroid interface in donors with AMD compared to donors without AMD. Particular attention was paid to genes known to participate in inflammation, immune-associated processes, coagulation/fibrinolysis and/or extracellular matrix homeostasis.

In choosing SNPs for these genes, a higher SNP density in the genic regions, which was defined as 5 Kb upstream from the start of transcription until 5 Kb downstream from the end of transcription, was applied. In these regions, an average density of 1 SNP per 10 Kb was used. In the non-genic regions of clusters of complement-related genes, an average of 1 SNP per 20 Kb was employed. The SNPs were chosen from HapMap data in the Caucasian population, the SNP Consortium (Marshall 1999 Science 284[5413]:406-407), Whitehead, NCBI and the Celera SNP database. Selection included intronic SNPs, variants from the regulatory regions (mainly promoters) and coding SNPs (cSNPs) included in open reading frames. Data obtained by direct screening were used to validate the information extracted from databases. Thus, the overall sequence variation of functionally important regions of candidate genes was investigated, not only on a few polymorphisms using a previously described algorithm for tag selection.

Positive controls included CEPH members (i.e., DNA samples derived from lymphoblastoid cell lines from 61 reference families provided to the NIGMS Repository by the Centre de'Etude du Polymorphism Humain (CEPH), Foundation Jean Dausset in Paris, France) of the HapMap trios; the nomenclature used for these samples is the Coriell sample name (i.e., family relationships were verified by the Coriell Institute for Medical Research Institute for Medical Research). The panel also contained a limited number of X-chromosome probes from two regions. These were included to provide additional information for inferring sample sex. Specifically, if the sample is clearly heterozygous for any X-chromosome markers, it must have two X-chromosomes. However, because there are a limited number of X-chromosome markers in the panel, and because their physical proximity likely means that there are even fewer haplotypes for these markers, we expected that samples with two X-chromosomes might also genotype as homozygous for these markers. The standard procedure for checking sample concordance involved two steps. The first step was to compare all samples with identical names for repeatability. In this study, the only repeats were positive controls and those had repeatability greater than 99.3% (range 99.85% to 100%). The second step was to compare all unique samples to all other unique samples and identify highly concordant sample pairs. Highly concordant sample pairs were used to identify possible tracking errors. The concordance test resulted in 20 sample pairs with concordance greater than 99%.

Samples were genotyped using multiplexed Molecular Inversion Probe (MIP) technology (ParAllele Biosciences/Affymetrix). Successful genotypes were obtained for 3,267 SNPs in 347 genes in 1113 unique samples (out of 1162 unique submitted samples; 3,267 successful assays out 3,308 assays attempted). SNPs with more than 5% failed calls (45 SNPs), SNPs with no allelic variation (354 alleles) and subjects with more than 5% missing genotypes (11 subjects) were deleted.

The resulting genotype data were analyzed in multiple sub-analyses, using a variety of appropriate statistical analyses, as described below.

A. Genes Associated with AMD

The study revealed a large number of new genes which were not previously reported to be associated with risk for, or protection from, AMD. These genes are shown in Tables I and include ADAM12, ADAM19, APBA2, APOB, BMP7, C1Qa, C1RL, C4BPA, C5, C8A, CCL28, CLU, COL9A1, FGFR2, HABP2, EMID2, COL6A3, IFNAR2, COL4A1, FBLN2, FBN2, FCN1, HS3ST4, IGLC1, IL12RB1, ITGAX, MASP1, MASP2, MYOC, PPID, PTPRC, SLC2A2, SPOCK, and TGFBR2. Additional genes that are associated with AMD are shown in Table II and include C3, C7, C9, C1NH, and ITGA4. The presence or absence of a polymorphism or variant in any one of these genes may be indicative of increased or decreased risk of AMD. In some embodiments, the presence or absence of a combination of polymorphisms in a combination of genes selected from Table I and/or II may be indicative of increased or decreased risk. In one embodiment, ADAM12 is associated with AMD and an individual's genetic profile comprises at least one SNP in ADAM12.

A short description of each of the genes identified in the study as associated with AMD is provided herein. In addition, gene identifiers based on the EnsEMBL database are provided in Table 5.

The ADAM12 (ADAM metallopeptidase domain 12, also known as MCMP, MLTN, MLTNA, and MCMPM1tna) gene is located at chromosome 10q26.3. ADAM12 is a member of the ADAM (a disintegrin and metalloprotease) protein family. Members of this family are membrane-anchored proteins structurally related to snake venom disintegrins, and have been implicated in a variety of biological processes involving cell-cell and cell-matrix interactions, including fertilization, muscle development, and neurogenesis. ADAM12 is known to cleave insulin-like growth factor binding proteins IGFBP-3 and IGFBP-5 as well as the heparin-binding epidermal growth factor (HB-EGF). Recently, it has been demonstrated that inhibitors of the ADAM12 processing of HB-EGF attenuate cardiac hypertrophy. ADAM12 has also been implicated in the differentiation of mesenchymal cells such as skeletal myoblasts and osteoblasts Inhibitors of ADAM12 may have therapeutic potential for the treatment of cardiac hypertrophy and cancer.

The ADAM19 (ADAM metallopeptidase domain 19, also known as MLTNB, FKSG34, and MADDAM) gene is located at chromosome 5q32-q33. ADAM19 is another member of the ADAM (a disintegrin and metalloprotease) protein family. ADAM19 was initially identified in muscle cells and was later found to be expressed in several other tissues, including the heart, lung, and bone, during dendritic cell differentiation, and Notch-induced T-cell maturation. ADAM19 has also been implicated in ectodomain shedding of neuregulin I-β, a protein that is essential for proper trabeculation of the heart during early development.

The APBA2 (Amyloid Beta (A4) Precursor Protein-Binding, Family A, Member 2, also known as X11L, MINT2, and LIN-10) gene is located at chromosome15q11-q12. APBA2 is a member of the X11 protein family. APBA2 is a neuronal adapter protein that interacts with the Alzheimer's disease amyloid precursor protein (APP). It stabilizes APP and inhibits production of proteolytic APP fragments including the A beta peptide that is deposited in the brains of Alzheimer's disease patients. APBA2 is also regarded as a putative vesicular trafficking protein in the brain that can form a complex with the potential to couple synaptic vesicle exocytosis to neuronal cell adhesion. Inhibitors of APBA2 may have therapeutic potential in treatments for Alzheimer's disease.

The ApoB (Apolipoprotein B, also known as ApoB-48 and ApoB-100) gene is located at chromosome 2p24-p23. ApoB is the main component of low density lipoprotein (LDL), chylomicrons and very low density lipoprotein (VLDL). ApoB occurs in the plasma in 2 main forms, ApoB48 and ApoB100. In humans, the first is synthesized exclusively in the gut by intestinal cells, and the second by the liver. ApoB100 is a component of VLDL, intermediate density lipoprotein (IDL) and low density lipoproteins (LDL) and contributes to hepatic and peripheral tissue uptake of LDL by receptor recognition. ApoB48 is an important component of chylomicrons and is required for their formation. Inhibitors of ApoB have been suggested as therapeutic targets for the treatment of atherosclerosis, hypertriglyceridemia, and/or hypercholesteremia.

The BMP7 (Bone Morphogenetic Protein 7, also known as OP-1) gene is located chromosome 20q13, and is expressed in the brain, kidneys, and bladder. BMP7 is a member of the bone morphogenetic protein (BMP) family. BMP proteins are secreted signaling molecules that play a key role in the transformation of mesenchymal cells into bone and cartilage. Many BMPs, including BMP7, are part of the transforming growth factor-beta (TGFB) superfamily. BMP7 is involved in bone homeostasis. BMP7 induces the phosphorylation of SMAD1 and SMAD5, which in turn induce transcription of numerous osteogenic genes. Human recombinant BMP7 is used to prevent neurologic trauma and in the treatment of tibial non-union, frequently in cases where a bone graft has failed. BMP7 also has the potential for treating chronic kidney disease and obesity.

The C1Qa (complement component 1, q subcomponent, A chain) gene is located at at chromosome 1p36.12 and encodes a major constituent of the human complement subcomponent C1q. C1q associates with C1r and C1s in order to yield the first component of the serum complement system. Deficiency of C1q has been associated with lupus erythematosus and glomerulonephritis. C1q is composed of 18 polypeptide chains: six A-chains, six B-chains, and six C-chains. Each chain contains a collagen-like region located near the N terminus and a C-terminal globular region. C1Qa is the A-chain polypeptide of human complement subcomponent C1q.

The C1RL (Complement Component 1, R Subcomponent-like, also known as C1RL1, C1RLP, CLSPa, and C1r-LP) gene is located at chromosome 12p13.31, and is expressed primarily in the liver. C1RL possesses protease activity and specifically cleaves pro-C1s into two fragments that are active C1s.

The C4BPA (Complement Component 4 Binding Protein, Alpha, also known as PRP and C4BP) gene is located on chromosome 1q32. C4BPA belong to a superfamily of proteins composed predominant of tandemly arrayed short consensus repeats of approximately 60 amino acids. Along with a single, unique beta-chain, seven identical alpha-chains encoded by this gene assemble into the predominant isoform of C4b-binding protein (C4BP), a multimeric protein that controls activation of the complement cascade through the classical pathway. C4BP inhibits the action of C4. It cleaves C4 convertase and is a cofactor for factor I, which cleaves C4b. C4BP also binds Cd40 on B cells which potentiates proliferation and costimulation.

The C5 (Complement Component 5) gene is located at chromosome 9q33-q34. C5 is the fifth component of complement, which plays an important role in inflammatory and cell killing processes. C5 is comprised of alpha and beta polypeptide chains that are linked by a disulfide bridge. C5a is derived from the alpha polypeptide via cleavage with a convertase, and is an anaphylatoxin that possesses potent spasmogenic and chemotactic activity. The C5b macromolecular cleavage product can form a complex with the C6 complement component, and this complex is the basis for formation of the membrane attack complex, which includes additional complement components. In certain embodiments, C5 inhibitors may be used for the treatment of, for example, sepsis, adult respiratory distress syndrome, and glomerulonephritis.

The C8A (Complement Component 8, Alpha Polypeptide) gene is located at chromosome 1p32. C8 is a component of the complement system and is comprised of three polypeptides, alpha (C8A), beta and gamma. C8 is one of five complement components (C5b, C6, C7, C8, and C9) that assemble on bacterial membranes to form a porelike structure referred to as the “membrane attack complex” (MAC). Membrane attack is important for mammalian immune defense against invading microorganisms and infected host cells.

The CCL28 (Chemokine (C—C motif) Ligand 28, also known as MEC, CCK1, and SCYA28) gene is located at chromosome 5p12. CCL28 belongs to the subfamily of small cytokine CC genes. CCL28 regulates the chemotaxis of cells that express the chemokine receptors CCR3 and CCR10. CCL28 is expressed by columnar epithelial cells in the gut, lung, breast and the salivary glands and drives the mucosal homing of T and B lymphocytes that express CCR10, and the migration of eosinophils expressing CCR3. This chemokine is constitutively expressed in the colon, but its levels can be increased by pro-inflammatory cytokines and certain bacterial products implying a role for CCL28 in effector cell recruitment to sites of epithelial injury. CCL28 has also been implicated in the migration of IgA-expressing cells to the mammary gland, salivary gland, intestine, and other mucosal tissues. It has also been shown as a potential antimicrobial agent effective against certain pathogens, such as Gram negative and Gram positive bacteria.

The CLU (Clusterin, also known as CLI, AAG4, APOJ, KUB1, SGP2, SP-40, and TRPM2) gene is located at chromosome 8p21-p12. CLU is a multifunctional glycoprotein that was first isolated from the male reproductive system. Subsequently, it has been shown that CLU is ubiquitously distributed among tissues, having a wide range of biologic properties. Among its many roles, CLU is a component of the soluble SCb-5 complement complex which is assembled in the plasma upon activation of the complement cascade. Binding of CLU has been shown to abolish the membranolytic potential of complement complexes and it has therefore been termed complement lysis inhibitor (CLI). Further investigations of CLU demonstrated that it circulates in plasma as a high density lipoprotein (HDL) complex, which serves not only as an inhibitor of the lytic complement cascade, but as a regulator of lipid transport and local lipid redistribution. CLU has also been shown to participate in the cellular process of programmed cell death or apoptosis. CLU expression demarcates cells undergoing apoptosis. In certain embodiments, CLU inhibitors may be used in the treatment of prostate cancer, renal cell cancer, and breast cancer.

The COL9A1 (Collagen, Type IX, Alpha 1, also known as MED and EDM6) gene is located at chromosome 6q12-q14. COL9A1 is one of the three alpha chains of type IX collagen, which is a component of hyaline cartilage and the vitreous body of the eye.

The FGFR2 (Fibroblast Growth Factor Receptor 2, also known as BEK, JWS, CEK3, CFD1, ECT1, KGFR, TK14, TK25, BFR-1, CD332, and K-SAM) gene is located at chromosome 10q26. FGFR2 is a member of the fibroblast growth factor receptor family. FGFR family members differ from one another in their ligand affinities and tissue distribution. A full-length FGFR protein consists of an extracellular region, composed of three immunoglobulin-like domains, a single hydrophobic membrane-spanning segment and a cytoplasmic tyrosine kinase domain. The extracellular portion of the protein interacts with fibroblast growth factors to activate a cascade of downstream signals that ultimately regulate mitogenesis and differentiation. FGFR2 is a high-affinity receptor for acidic, basic and/or keratinocyte growth factor, depending on the isoform.

The HABP2 (Hyaluronan Binding Protein 2, also known as FSAP, HABP, PHBP, and HGFAL) gene is located at chromosome 10q25.3. HABP2 is an extracellular serine protease that binds hyaluronic acid and is involved in cell adhesion. HABP2 is involved with hemostasis by cleaving the urinary plasminogen activator, coagulation factor VII, and the alpha and beta chains of fibrinogen. HABP2 is also involved in the inhibition of vascular smooth muscle cell (VSMC) proliferation and migration as well as neointima formation.

The EMID2 (EMI Domain Containing 2, also known as EMI6, EMU2, and COL26A1) gene is located at chromosome 7q22.1. EMID2 was first isolated as a gene involved in early kidney development. Biochemical studies showed that EMID2 is a glycosylated protein that is secreted into the extracellular space, where it forms homo- or heterodimers. EMID2 expression is restricted to mesenchymal cells in tissues such as the kidney, salivary gland, and skeletal muscle.

The COL6A3 (Collagen, Type VI, Alpha 3) gene is located at chromosome 2q37. COL6A3 is one of the three alpha chains of type VI collagen, a beaded filament collagen found in most connective tissues. The alpha 3 chain of type VI collagen is much larger than the alpha 1 and 2 chains. This difference in size is largely due to an increase in the number of subdomains found in the amino terminal globular domain of all the alpha chains. These domains have been shown to bind extracellular matrix proteins, an interaction critical for the function of this collagen in organizing matrix components.

The IFNAR2 (interferon [alpha, beta and omega] receptor 2, also known as IFN-R; IFNABR; IFNARB; IFN-alpha-REC) gene is located at chromosome 21q22.11/q22.1 and encodes a type I membrane protein that forms one of the two chains of a receptor for interferons alpha and beta. Binding and activation of the receptor stimulates Janus protein kinases, which in turn phosphorylate several proteins, including STAT1 and STAT2. They are potent inhibitors of type I IFN activity. The IRNAR2 protein has been reported to highly expressed in liver, kidney, peripheral blood B cells and monocytes.

The COL4A1 (Collagen, type IV, alpha 1, also known as Arresten) gene is located at chromosome 13q34 and encodes one of the six type IV collagen isoforms, alpha 1(IV)-alpha 6(IV), each of which can form a triple helix structure with 2 other chains to generate type IV collagen. Type IV collagen is the major structural component of glomerular basement membranes (GBM), forming a “chicken-wire” meshwork together with laminins, proteoglycans and entactin/nidogen. It potently inhibits endothelial cell proliferation and angiogenesis, potentially via mechanisms involving cell surface proteoglycans and the alpha and beta integrins of endothelial cells. Like the other members of the type IV collagen gene family, this gene is organized in a head-to-head conformation with another type IV collagen gene so that each gene pair shares a common promoter.

The FBLN2 (Fibulin 2) gene is located at chromosome 3p25.1 and encodes an extracellular matrix protein that belongs to the fibulin protein family. The FBLN2 protein has been found abundantly distributed in elastic fibers in many tissues, and is prominently expressed during morphogenesis of the heart and aortic arch vessels and at early stages of cartilage development. It may play a role during organ development, in particular, during the differentiation of heart, skeletal and neuronal structures.

The FBN2 (Fibrillin 2, also known as CCA and DA9) gene is located at chromosome 5q23-q31. FBN2 is a member of the fibrillin protein family. Fibrillin proteins are large glycoproteins that are structural components of 10-12 nm extracellular calcium-binding microfibrils, which occur either in association with elastin or in elastin-free bundles. FBN2-containing microfibrils may regulate the early process of elastic fiber assembly.

The FCN1 (Ficolin [collagen/fibrinogen domain containing] 1, also known as FCNM) gene is located at chromosome 9q34. FCN1 is a member of the ficolin protein family. The ficolin family of proteins are characterized by the presence of a leader peptide, a short N-terminal segment, followed by a collagen-like region, and a C-terminal fibrinogen-like domain. However, all these proteins recognize different targets, and are functionally distinct. Ficolin 1 encoded by the FCN1 gene is predominantly expressed in the peripheral blood leukocytes, and may function as a plasma protein with elastin-binding activity.

The HS3ST4 (heparan sulfate [glucosamine] 3-O-sulfotransferase 4, also known as 30ST4; 30ST4; 3-OST-4) gene is located at chromosome 16p11.2. HS3ST4 is one of the isoforms of the enzyme heparan sulfate D-glucosaminyl 3-O-sulfotransferase. This enzyme generates 3-O-sulfated glucosaminyl residues in heparan sulfate. Cell surface heparan sulfate is used as a receptor by herpes simplex virus type 1 (HSV-1), and the HS3ST4 protein is thought to play a role in HSV-1 pathogenesis. It is primarily expressed in brain.

The IGLC1 (immunoglobulin lambda constant 1 [Mcg marker], also known as IGLC) gene is located at chromosome 22q11.2 and encodes the immunoglobulin lambda chain.

IL12RB1 (interleukin 12 receptor, beta 1, also known as CD212; IL12RB; MGC34454; IL-12R-BETA1) gene is located at chromosome 19p13.1 and encodes a type I transmembrane protein that belongs to the hemopoietin receptor superfamily. The IL12RB1 protein binds to interleukin 12 (IL12) with a low affinity, and is thought to be a part of an IL12 receptor complex. This protein forms a disulfide-linked oligomer, which is required for its IL12 binding activity. The coexpression of this and IL12RB2 proteins was shown to lead to the formation of high-affinity IL12 binding sites and reconstitution of IL12 dependent signaling. The lack of expression of this gene was found to result in the immunodeficiency of patients with severe mycobacterial and Salmonella infections.

The ITGAX (integrin, alpha X [complement component 3 receptor 4 subunit], also known as CD11C) gene is located at chromosome 16p11.2 and encodes an alpha X chain of Integrins, which are heterodimeric integral membrane proteins composed of an alpha chain and a beta chain. The alpha X chain associates with beta 2 chain to form a leukocyte-specific integrin referred to as inactivated-C3b (iC3b) receptor 4 (CR4). The intergrin alpha X/beta 2 is involved in the adherence of neutrophils and monocytes to stimulated endothelium cells, and in the phagocytosis of complement coated particles. ITGAX is found primarily on myeloid cells, where its expression is regulated both during differentiation and during monocyte maturation into tissue macrophages.

The MASP1 (mannan-binding lectin serine peptidase 1 [C4/C2 activating component of Ra-reactive factor], also known as MASP, RaRF, CRARF, PRSS5, CRARF1, FLJ26383, MGC126283, MGC126284, and DKFZp686I01199) gene is located at chromosome 3q27-q28. MASP1 is a member of the mannan-binding lectin (MBL) associated serine proteases (MASPs), which are involved in the lectin pathway of the complement system. Mannose-binding lectin (MBL) is an oligomeric serum lectin. In the lectin pathway, MBL and serum ficolins bind directly to sugars or N-acetyl groups on pathogenic cells and activate the MASPs, which then trigger the activation of complement cascade by activating the C4 and C2 components. In the lectin pathway, the MASP1 protein cleaves the C2 component.

The MASP2 (mannan-binding lectin serine peptidase 1 [C4/C2 activating component of Ra-reactive factor], also known as sMAP, MAP19, and MASP-2) gene is located at chromosome 1p36.2-p36.3. MASP2 is another member of the mannan-binding lectin (MBL) associated serine proteases (MASPs), which are involved in the lectin pathway of the complement system. Mannose-binding lectin (MBL) is an oligomeric serum lectin. In the lectin pathway, MBL and serum ficolins bind directly to sugars or N-acetyl groups on pathogenic cells and activate the MASPs, which then trigger the activation of complement cascade by activating the C4 and C2 components. In the lectin pathway, the MASP2 protein cleaves the C4 and C2 components, leading to their activation and to the formation of C3 convertase.

The MYOC (myocilin, trabecular meshwork inducible glucocorticoid response, also known as GPOA, JOAG, TIGR, GLC1A and JOAG1) gene is located at chromosome 1q23-24 and encodes a protein that has a role in cytoskeletal function. The MYOC protein is expressed in many occular tissues, including the trabecular meshwork, which is a specialized eye tissue essential in regulating intraocular pressure. The unprocessed myocilin with signal peptide is a 55-kDa protein with 504 amino acids. Mature myocilin is known to form multimers. Wild type myocilin protein is normally secreted into the trabecular extracellular matrix (ECM) and there appears to interact with various ECM materials. The deposition of high amounts of myocilin in trabecular ECM could affect aqueous outflow either by physical barrier and/or through cell-mediated process leading to elevation of IOP. The MYOC protein is also the trabecular meshwork glucocorticoid-inducible response protein (TIGR). Mutations in MYOC have been identified as the cause of hereditary juvenile-onset open-angle glaucoma.

The PPID (peptidylprolyl isomerase D, also known as CYPD; CYP-40; MGC33096) gene is located at chromosome 4q31.3 and encodes a member of the peptidyl-prolyl cis-trans isomerase (PPIase) family. PPIases catalyze the cis-trans isomerization of proline imidic peptide bonds in oligopeptides and accelerate the folding of proteins. The PPID protein possess PPIase activity and, similar to other family members, can bind to the immunosuppressants cyclosporin A. The PPID protein is a key factor in the regulation of a mitochondrial protein complex called the permeability transition pore, which mediates the permeabilization of the mitochondrial membrane and cytochrome c release and is involved in the process of apoptosis. Overexpress of the PPID protein suppresses apoptosis.

The PTPRC (protein tyrosine phosphatase, receptor type, C, also known as LCA; LY5; B220, CD45, T200, CD45R, and GP180) gene is located at chromosome 1q31-q32. PTPRC is a member of the protein tyrosine phosphatase (PTP) family. PTPs are known to be signaling molecules that regulate a variety of cellular processes including cell growth, differentiation, mitotic cycle, and oncogenic transformation. It is specifically expressed in hematopoietic cells. The PTP protein encoded by the PTPRC gene contains an extracellular domain, a single transmembrane segment and two tandem intracytoplasmic catalytic domains, and thus belongs to receptor type PTP. The PTPRC protein has also been shown to be an essential regulator of T- and B-cell antigen receptor signaling. It also functions through either direct interaction with components of the antigen receptor complexes, or by activating various Src family kinases required for the antigen receptor signaling. The PTPRC protein also suppresses JAK kinases, and thus functions as a regulator of cytokine receptor signaling.

The SLC2A2 (solute carrier family 2 [facilitated glucose transporter], member 2, also known as GLUT2) gene is located at chromosome 3q26.1-q26.2 and encodes an integral plasma membrane glycoprotein. It is expressed in the liver, islet beta cells, intestine, and kidney epithelium. The SLC2A2 protein mediates facilitated bidirectional transportation of glucose across the plasma membrane of hepatocytes and is responsible for uptake of glucose by the beta cells. It may comprise part of the glucose-sensing mechanism of the beta cell, and may also participate with the Na(+)/glucose cotransporter in the transcellular transport of glucose in the small intestine and kidney.

The SPOCK (sparc/osteonectin, cwcv and kazal-like domains proteoglycan [testican] 1, also known as TIC1 and FLJ37170) gene is located at chromosome 5q31 and encodes the protein core of a plasma proteoglycan containing chondroitin- and heparan-sulfate chains. The function of the SPOCK protein is unknown, although similarity to thyropin-type cysteine protease-inhibitors suggests its function may be related to protease inhibition. The SPOCK gene is primarily expressed in brain.

The TGFBR2 (transforming growth factor, beta receptor II (70/80 kDa), also known as AAT3, FAA3, MFS2, RIIC, LDS1B, LDS2B, TAAD2, TGFR-2, and TGFbeta-RII) gene is located at chromosome 3p22. TGFBR2 is a member of the Ser/Thr protein kinase family and the transforming growth factor-beta (TGFB) receptor subfamily. The encoded protein is a transmembrane protein that has a protein kinase domain, forms a heterodimeric complex with another receptor protein, and binds TGF-beta. This receptor/ligand complex phosphorylates proteins, which then enter the nucleus and regulate the transcription of a subset of genes related to cell proliferation. Mutations in this gene have previously been associated with Marfan Syndrome, Loeys-Deitz Aortic Aneurysm Syndrome, and the development of various types of tumors.

Additional genes that are associated with AMD include C3, C7, C9, C1NH, and ITGA4. A brief summary of the biological function of each of these genes is provided below.

The C3 (complement component 3, also known as ASP, ARMD9, and CPAMD1) gene is located at chromosome 19p13.3-p13.2 and encodes an essential protein of the immune system. The C3 protein plays a central role in the complement system and contributes to innate immunity. Its activation is required for both classical and alternative complement activation pathways. Soluble C3-convertase, also known as C4b2a, catalyzes the proteolytic cleavage of C3 into C3a and C3b as part of the classical complement system as well as the lectin pathway. C3a is an anaphylotoxin, and C3b serves as an opsonizing agent. Factor I can cleave C3b into C3c and C3d, the latter of which plays a role in enhancing B cell responses. In the alternative complement pathway, C3 is cleaved by iC3Bb, another form of C3-convertase.

The C7 (complement component 7) gene is located at chromosome 5p13 and encodes a component of the complement system. It participates in the formation of the complement Membrane Attack Complex (MAC), which is a large, membrane-bound protein complex that performs the cell lysis function of complement.

The C9 (complement component 9) gene is located at chromosome 5p14-p12 and encodes a component of the complement system. It is the final component of the complement system to be added in the assembly of the complement Membrane Attack Complex (MAC), which is a large, membrane-bound protein complex that performs the cell lysis function of complement.

The C1NH(C1 Inhibitor, also known as SERPING1, C1IN, C1-INH, HAE1, and HAE2) gene is located at chromosome 11q12-q13.1. C1NH is a highly glycosylated plasma protein involved in the regulation of the complement cascade. C1NH is a serine protease inhibitor protein that inhibits the complement system to prevent spontaneous activation. The C1NH protein inhibits activated C1r and C1s of the first complement component, and thus regulates complement activation. Although named after its complement inhibitory activity, C1NH also inhibits proteinases of the fibrinolytic, clotting, and kinin pathways. Most notably, C1NH is also the physiological inhibitor of plasma kallikrein, fXIa and fXIIa. In some embodiments, recombinant C1NH may be used for the treatment of hereditary angioneurotic edema (HANE) and heart attack by preventing the activation of the complement cascade.

The ITGA4 (integrin, alpha 4 [antigen CD49D, alpha 4 subunit of VLA-4 receptor], also known as IA4; CD49D; MGC90518) gene is located at chromosome 2q31.3. It encodes an alpha 4 chain of an integrin, which is a heterodimeric integral membrane protein composed of an alpha chain and a beta chain. The alpha 4 chain associates with a beta 1 chain or beta 7 chain. Integrins alpha-4/beta-1 (VLA-4) and alpha-4/beta-7 are receptors for fibronectin and VCAM1. Integrin alpha-4/beta-7 is also a receptor for MADCAM1. On activated endothelial cells, integrin VLA-4 triggers homotypic aggregation for most VLA-4-positive leukocyte cell lines. It may also participate in cytolytic T-cell interactions with target cells. Integrin VLA-4 is expressed on monocytes, lymphocytes and at a low level on neutrophils, and supports both slow rolling and firm adhesion to the activated endothelium via ligation of VCAM-1 or fibronectin. Deletion of the VLA-4 (ITGA4) gene results in fetal death.

### B. Polymorphisms Associated with AMD

One genotype association analysis was performed on all SNPs comparing samples derived from patients with AMD to those derived from an age-matched control cohort. All genotype associations were assessed using a statistical software program known as SAS®. SNPs showing significant association with AMD are shown in Table I. Tables I and II include SNPs from CCL28, FBN2, ADAM12, PTPRC, IGLC1, HS3ST4, PRELP, PPID, SPOCK, APOB, SLC2A2, COL4A1, COL6A3, MYOC, ADAM19, FGFR2, C8A, FCN1, IFNAR2, C1NH, C7, and ITGA4, with additional raw data provided in Tables III and IV as discussed in greater detail hereinbelow. Table VI includes SNPs from the RCA locus from FHR1 through F13B.

The genotypes depicted in Tables are organized by gene symbol. AMD associated SNPs identified in a given gene are designated by SNP number or MRD designation. For each SNP, allele frequencies are shown as percentages in both control and disease (AMD) populations. Allele frequencies are provided for individuals homozygous for allele 1 and allele 2, and for heterozygous individuals. For example, for SNP rs1676717, which is located in ADAM metallopeptidase domain 12 (ADAM12), 17.6% of the control population is homozygous for allele 1 (i.e., the individual has a “A” base at this position), 29% of the control population is homozygous for allele 2 (i.e., the individual has a “G” base at this position), and 53.4% of the control population is heterozygous. The overall frequency for allele 1 (i.e., the “A” allele) in the control population is 44.3% and the overall frequency for allele 2 (i.e., the “G” allele) in the control population is 55.7%. In the AMD population, 13.5% of the population is homozygous for allele 1 (the “A” allele), 41.2% of population is homozygous for allele 2 (the “G” allele), and 45.3% of the population is heterozygous. The overall frequency for allele 1 (the “A” allele) in the AMD population is 36.1% and the overall frequency for allele 2 (the “G” allele) in the AMD population is 63.9%. Genotype likelihood ratios (3 categories; genotype p value) and Chi Square (“Freq. Chi Square (both collapsed-2 categories)”) values are provided for each SNP. Tables VII and VIII provide the nucleotide sequences flanking the SNPs disclosed in Tables I and II. For each sequence, the “N” refers to the polymorphic site. The nucleotide present at the polymorphic site is either allele 1 or allele 2 as shown in Tables I and II.

In some cases in Table I, “MRD” designations are provided in place of SNP number designations. MRD_{—}4048 corresponds to the following sequence AGCTTCGATATGACTCCACCTGTGAACGTCT(C/G)TACTATGGAGATGATGAGAA ATACTTTCGGA, which is the region flanking the SNP present in the C8A gene: (SEQ ID NO: 1). MRD_{—}4044 corresponds to the following sequence AGGAGAGTAAGACGGGCAGCTACACCCGCAG(A/C)AGTTACCTGCCAGCTGAGC AACTGGTCAGAG, which is the region flanking the SNP present in the C8A gene: (SEQ ID NO: 2). MRD_{—}4452 corresponds to the following sequence GCGTGGTCAGGGGCTGAGTTTTCCAGTTCAG(A/G)ATCAGGACTATGGAGGCACA ACATGGAGGCC, which is the region flanking the SNP present in the CLU gene: (SEQ ID NO: 3). The polymorphic site indicating the SNP associated alleles are shown in parentheses. Further, certain SNPs presented in Table I were previously identified by MRD designations in provisional application, U.S. Application No. 60/984,702. For example, the SNP designated rs2511988 is also called MRD_{—}4083; the SNP designated rs172376 is also called MRD_{—}4035; the SNP designated rs61917913 is also called MRD_{—}4110; the SNP designated rs2230214 is also called MRD_{—}4475; the SNP designated rs10985127 is also called MRD_{—}4477; the SNP designated rs10985126 is also called MRD_{—}4476; the SNP designated rs7857015 is also called MRD_{—}4502; the SNP designated rs3012788 is also called MRD_{—}4495; the SNP designated rs2230429 is also called MRD_{—}4146; the SNP designated rs12142107 is also called MRD_{—}3848; the SNP designated rs2547438 is also called MRD_{—}4273; the SNP designated rs2230199 is also called MRD_{—}4274; the SNP designated rs1047286 is also called MRD_{—}4270; and the SNP designated rs11085197 is also called MRD_{—}4269.

The presence in the genome or the transcriptome of an individual of one or more polymorphisms listed in Tables I and/or II is associated with an increased or decreased risk of AMD. Accordingly, the detection of a polymorphism shown in Tables I and/or II in a nucleic acid sample of an individual can indicate that the individual is at increased risk for developing AMD. One of skill in the art will be able to refer to Table Ito identify alleles associated with increased (or decreased) likelihood of developing AMD. For example, in the gene ADAM12, allele 2 of the SNP rs1676717 is found in 63.9% of AMD chromosomes, but only in 55.7% of the control chromosomes indicating that a person having allele 2 has a greater likelihood of developing AMD than a person not having allele 2 (See Table I). Allele 2 (“G”) is the more common allele (i.e. the “wild type” allele). The “A” allele is the rarer allele, but is more prevalent in the control population than in the AMD population: it is therefore a “protective polymorphism.” Table III(A-B) provides the raw data from which the percentages of allele frequencies as shown in Table I were calculated. Table III(C) depicts the difference in percentage allele frequence in homozygotes for allele 1 and allele 2 between control and disease populations, the difference in percentage allele frequency in heterozygotes between control and disease populations, and the difference in percentage for undetermined subjects between control and disease populations.

Table II provides additional genes and single nucleotide polymorphisms that were discovered to be associated with AMD. As described for Table I, the genotypes depicted in Table II are organized by gene symbol. For each SNP, allele frequencies are presented as percentages in by control and disease populations. Allele frequencies are shown for individuals homozygous for allele 1 and allele 2, and heterozygous individuals. Genotype-likelihood ratios and Chi square values are provided for each SNP. Table IV (A-B) provides the raw data from which the percentages of allele frequencies shown in Table II were calculated. Table IV(C) depicts the difference in percentage allele frequency in homozygotes for allele 1 and allele 2 between control and disease populations, the difference in percentage allele frequency in heterozygotes between control and disease populations, and the difference in percentage for undetermined subjects between control and disease populations.

In other embodiments, the presence of a combination of multiple (e.g., two or more, or three or more, four or more, or five or more) AMD-associated polymorphisms shown in Tables I and/or II indicates an increased (or decreased) risk for AMD.

In addition to the new AMD SNP associations defined herein, these experiments confirmed previously reported associations of AMD with variations/SNPs in the CFH, FHR1-5, F13B, LOC387715, PLEKHA1 and PRSS11 genes.

### V. Determination of Risk (Screening)

### Determining the Risk of an Individual

An individual's relative risk (i.e., susceptibility or propensity) of developing a particular complement-related disease can be determined by screening for the presence or absence of a genetic profile in at least one of the genes shown in Table I and/or II. In a preferred embodiment, the complement-related disease is AMD.

A genetic profile for AMD comprises one or more single nucleotide polymorphisms (SNPs) selected from Tables I and/or II. The presence of any one of the SNPs listed in Table I and/or II is informative (i.e., indicative) of an individual's increased or decreased risk of developing AMD or for predicting the course of progression of AMD in the individual (i.e., a patient).

In one embodiment, the predictive value of a genetic profile for AMD can be increased by screening for a genetic profile in two or more, three or more, four or more, or five or more genes selected from Tables I and/or II.

In another embodiment, the predictive value of a genetic profile for AMD can be increased by screening for a combination of SNPs selected from Tables I and/or II, typically from multiple genes. In one embodiment, predictive value of a genetic profile is increased by screening for the presence of at least 2 SNPs, at least 3 SNPs, at least 4 SNPs, at least 5 SNPs, at least 6 SNPs, at least 7 SNPs, at least 8 SNPS, at least 9 SNPs, or at least 10 SNPs selected from Tables I and/or II, typically from multiple genes. In another embodiment, the predictive value of a genetic profile for AMD is increased by screening for the presence of at least one SNP from Tables I and/or II and at least one additional SNP selected from the group consisting of a polymorphism in exon 22 of CFH (R1210C), rs1061170, rs203674, rs1061147, rs2274700, rs12097550, rs203674, rs9427661, rs9427662, rs10490924, rs11200638, rs2230199, rs800292, rs3766404, rs529825, rs641153, rs4151667, rs547154, rs9332739, rs2511989, rs3753395, rs1410996, rs393955, rs403846, rs1329421, rs10801554, rs12144939, rs12124794, rs2284664, rs16840422, and rs6695321. In certain embodiments, the method may comprise screening for at least one SNP from Tables I and/or II and at least one additional SNP associated with risk of AMD selected from the group consisting of: a polymorphism in exon 22 of CFH(R1210C), rs1061170, rs203674, rs1061147, rs2274700, rs12097550, rs203674, rs9427661, rs9427662, rs10490924, rs11200638, and rs2230199.

The predictive value of a genetic profile for AMD can also be increased by screening for a combination of predisposing and protective polymorphisms. For example, the absence of at least one, typically multiple, predisposing polymorphisms and the presence of at least one, typically multiple, protective polymorphisms may indicate that the individual is not at risk of developing AMD. Alternatively, the presence of at least one, typically multiple, predisposing SNPs and the absence of at least one, typically multiple, protective SNPs indicate that the individual is at risk of developing AMD. In one embodiment, a genetic profile for AMD comprises screening for the presence of at least one SNP selected from Tables I and/or II and the presence or absence of at least one protective SNP selected from the group consisting of: rs800292, rs3766404, rs529825, rs641153, rs4151667, rs547154, and rs9332739.

In some embodiments, the genetic profile for AMD includes at least one SNP from ADAM12. In one embodiment, the at least one SNP includes rs1676717. In one embodiment, the at least one SNP includes rs1621212. In one embodiment, the at least one SNP includes rs12779767. In one embodiment, the at least one SNP includes rs11244834.

In some embodiments, the genetic profile for AMD includes at least one SNP from ADAM19. In one embodiment, the at least one SNP includes rs12189024. In one embodiment, the at least one SNP includes rs7725839. In one embodiment, the at least one SNP includes rs11740315. In one embodiment, the at least one SNP includes rs7719224. In one embodiment, the at least one SNP includes rs6878446.

In some embodiments, the genetic profile for AMD includes at least one SNP from APBA2. In one embodiment, the at least one SNP includes rs3829467.

In some embodiments, the genetic profile for AMD includes at least one SNP from APOB. In one embodiment, the at least one SNP includes rs12714097.

In some embodiments, the genetic profile for AMD includes at least one SNP from BMP7. In one embodiment, the at least one SNP includes rs6014959. In one embodiment, the at least one SNP includes rs6064517. In one embodiment, the at least one SNP includes rs162315. In one embodiment, the at least one SNP includes rs162316.

In some embodiments, the genetic profile for AMD includes at least one SNP from C1Qa. In one embodiment, the at least one SNP includes rs172376.

In some embodiments, the genetic profile for AMD includes at least one SNP from C1RL. In one embodiment, the at least one SNP includes rs61917913.

In some embodiments, the genetic profile for AMD includes at least one SNP from C4BPA. In one embodiment, the at least one SNP includes rs2842706. In one embodiment, the at least one SNP includes rs1126618.

In some embodiments, the genetic profile for AMD includes at least one SNP from C5. In one embodiment, the at least one SNP includes rs7033790. In one embodiment, the at least one SNP includes rs10739585. In one embodiment, the at least one SNP includes rs2230214. In one embodiment, the at least one SNP includes rs10985127. In one embodiment, the at least one SNP includes rs2300932. In one embodiment, the at least one SNP includes rs12683026. In one embodiment, the at least one SNP includes rs4837805.

In some embodiments, the genetic profile for AMD includes at least one SNP from C8A. In one embodiment, the at least one SNP includes MRD_{—}4048. In one embodiment, the at least one SNP includes MRD_{—}4044.

In some embodiments, the genetic profile for AMD includes at least one SNP from CCL28. In one embodiment, the at least one SNP includes rs7380703. In one embodiment, the at least one SNP includes rs11741246. In one embodiment, the at least one SNP includes rs4443426.

In some embodiments, the genetic profile for AMD includes at least one SNP from CLU. In one embodiment, the at least one SNP includes MRD_{—}4452.

In some embodiments, the genetic profile for AMD includes at least one SNP from COL9A1. In one embodiment, the at least one SNP includes rs1135056.

In some embodiments, the genetic profile for AMD includes at least one SNP from FGFR2. In one embodiment, the at least one SNP includes rs2981582. In one embodiment, the at least one SNP includes rs2912774. In one embodiment, the at least one SNP includes rs1319093. In one embodiment, the at least one SNP includes rrs10510088. In one embodiment, the at least one SNP includes rs12412931.

In some embodiments, the genetic profile for AMD includes at least one SNP from HABP2. In one embodiment, the at least one SNP includes rs3740532. In one embodiment, the at least one SNP includes rs7080536.

In some embodiments, the genetic profile for AMD includes at least one SNP from EMID2. In one embodiment, the at least one SNP includes rs17135580. In one embodiment, the at least one SNP includes rs12536189. In one embodiment, the at least one SNP includes rs7778986. In one embodiment, the at least one SNP includes rs11766744.

In some embodiments, the genetic profile for AMD includes at least one SNP from COL6A3. In one embodiment, the at least one SNP includes rs4663722. In one embodiment, the at least one SNP includes rs1874573. In one embodiment, the at least one SNP includes rs12992087.

In some embodiments, the genetic profile for AMD includes at least one SNP from IFNAR2. In one embodiment, the at least one SNP includes rs2826552.

In some embodiments, the genetic profile for AMD includes at least one SNP from COL4A1. In one embodiment, the at least one SNP includes rs7338606. In one embodiment, the at least one SNP includes rs11842143. In one embodiment, the at least one SNP includes rs595325. In one embodiment, the at least one SNP includes rs9301441. In one embodiment, the at least one SNP includes rs754880. In one embodiment, the at least one SNP includes rs7139492. In one embodiment, the at least one SNP includes rs72509.

In some embodiments, the genetic profile for AMD includes at least one SNP from FBLN2. In one embodiment, the at least one SNP includes rs9843344. In one embodiment, the at least one SNP includes rs1562808.

In some embodiments, the genetic profile for AMD includes at least one SNP from FBN2. In one embodiment, the at least one SNP includes rs10057855. In one embodiment, the at least one SNP includes rs10057405. In one embodiment, the at least one SNP includes rs331075. In one embodiment, the at least one SNP includes rs17676236. In one embodiment, the at least one SNP includes rs6891153. In one embodiment, the at least one SNP includes rs17676260. In one embodiment, the at least one SNP includes rs154001. In one embodiment, the at least one SNP includes rs3805653. In one embodiment, the at least one SNP includes rs3828661. In one embodiment, the at least one SNP includes rs11241955. In one embodiment, the at least one SNP includes rs6882394. In one embodiment, the at least one SNP includes rs432792. In one embodiment, the at least one SNP includes rs13181926.

In some embodiments, the genetic profile for AMD includes at least one SNP from FCN1. In one embodiment, the at least one SNP includes rs10117466. In one embodiment, the at least one SNP includes rs7857015. In one embodiment, the at least one SNP includes rs2989727. In one embodiment, the at least one SNP includes rs3012788.

In some embodiments, the genetic profile for AMD includes at least one SNP from HS3ST4. In one embodiment, the at least one SNP includes rs4441276. In one embodiment, the at least one SNP includes rs12921387.

In some embodiments, the genetic profile for AMD includes at least one SNP from IGLC1. In one embodiment, the at least one SNP includes rs1065464. In one embodiment, the at least one SNP includes rs4820495.

In some embodiments, the genetic profile for AMD includes at least one SNP from IL12RB1. In one embodiment, the at least one SNP includes rs273493.

In some embodiments, the genetic profile for AMD includes at least one SNP from ITGAX. In one embodiment, the at least one SNP includes rs2230429. In one embodiment, the at least one SNP includes rs11574630.

In some embodiments, the genetic profile for AMD includes at least one SNP from MASP1. In one embodiment, the at least one SNP includes rs12638131.

In some embodiments, the genetic profile for AMD includes at least one SNP from MASP2. In one embodiment, the at least one SNP includes rs12142107.

In some embodiments, the genetic profile for AMD includes at least one SNP from MYOC. In one embodiment, the at least one SNP includes rs2236875. In one embodiment, the at least one SNP includes rs12035960. In one embodiment, the at least one SNP includes rs235868.

In some embodiments, the genetic profile for AMD includes at least one SNP from PPID. In one embodiment, the at least one SNP includes rs8396. In one embodiment, the at least one SNP includes rs7689418.

In some embodiments, the genetic profile for AMD includes at least one SNP from PTPRC. In one embodiment, the at least one SNP includes rs1932433. In one embodiment, the at least one SNP includes rs17670373. In one embodiment, the at least one SNP includes rs10919560.

In some embodiments, the genetic profile for AMD includes at least one SNP from SLC2A2. In one embodiment, the at least one SNP includes rs7646014. In one embodiment, the at least one SNP includes rs1604038. In one embodiment, the at least one SNP includes rs5400. In one embodiment, the at least one SNP includes rs11721319.

In some embodiments, the genetic profile for AMD includes at least one SNP from SPOCK. In one embodiment, the at least one SNP includes rs1229729. In one embodiment, the at least one SNP includes rs1229731. In one embodiment, the at least one SNP includes rs2961633. In one embodiment, the at least one SNP includes rs2961632. In one embodiment, the at least one SNP includes rs12656717.

In some embodiments, the genetic profile for AMD includes at least one SNP from TGFBR2. In one embodiment, the at least one SNP includes rs4955212. In one embodiment, the at least one SNP includes rs1019855. In one embodiment, the at least one SNP includes rs2082225. In one embodiment, the at least one SNP includes rs9823731.

In some embodiments, the genetic profile for AMD includes at least one SNP from C3. In one embodiment, the at least one SNP includes rs2547438. In one embodiment, the at least one SNP includes rs2230199. In one embodiment, the at least one SNP includes rs1047286. In one embodiment, the at least one SNP includes rs3745567. In one embodiment, the at least one SNP includes rs11569507. In one embodiment, the at least one SNP includes rs11085197.

In some embodiments, the genetic profile for AMD includes at least one SNP from C7. In one embodiment, the at least one SNP includes rs2271708. In one embodiment, the at least one SNP includes rs1055021.

In some embodiments, the genetic profile for AMD includes at least one SNP from C9. In one embodiment, the at least one SNP includes rs476569.

In some embodiments, the genetic profile for AMD includes at least one SNP from C1NH. In one embodiment, the at least one SNP includes rs4926. In one embodiment, the at least one SNP includes rs2511988. In one embodiment, the at least one SNP includes rs11740315.

In some embodiments, the genetic profile for AMD includes at least one SNP from ITGA4. In one embodiment, the at least one SNP includes rs3770115. In one embodiment, the at least one SNP includes rs4667319.

Although the predictive value of the genetic profile can generally be enhanced by the inclusion of multiple SNPs, no one of the SNPs is indispensable. Accordingly, in various embodiments, one or more of the SNPs is omitted from the genetic profile.

In certain embodiments, the genetic profile comprises a combination of at least two SNPs selected from the pairs of genes identified below:

Exemplary pairwise combinations of informative SNPs

| | ADAM12 | ADAM19 | APBA2 | APOB | BMP7 | C1NH | C1Qa | C1RL | C4BPA | C5 | C8A | C9 | CCL28 | CLU |
|---|---|---|---|---|---|---|---|---|---|---|---|---|---|---|
| ADAM12 | | X | X | X | X | X | X | X | X | X | X | X | X | X |
| ADAM19 | X | | X | X | X | X | X | X | X | X | X | X | X | X |
| APBA2 | X | X | | X | X | X | X | X | X | X | X | X | X | X |
| APOB | X | X | X | | X | X | X | X | X | X | X | X | X | X |
| BMP7 | X | X | X | X | | X | X | X | X | X | X | X | X | X |
| C1NH | X | X | X | X | X | | X | X | X | X | X | X | X | X |
| C1Qa | X | X | X | X | X | X | | X | X | X | X | X | X | X |
| C1RL | X | X | X | X | X | X | X | | X | X | X | X | X | X |
| C4BPA | X | X | X | X | X | X | X | X | | X | X | X | X | X |
| C5 | X | X | X | X | X | X | X | X | X | | X | X | X | X |
| C8A | X | X | X | X | X | X | X | X | X | X | | X | X | X |
| C9 | X | X | X | X | X | X | X | X | X | X | X | | X | X |
| CCL28 | X | X | X | X | X | X | X | X | X | X | X | X | | X |
| CLU | X | X | X | X | X | X | X | X | X | X | X | X | X | |
| COL9A1 | X | X | X | X | X | X | X | X | X | X | X | X | X | X |
| FGFR2 | X | X | X | X | X | X | X | X | X | X | X | X | X | X |
| HABP2 | X | X | X | X | X | X | X | X | X | X | X | X | X | X |
| EMID2 | X | X | X | X | X | X | X | X | X | X | X | X | X | X |
| COL6A3 | X | X | X | X | X | X | X | X | X | X | X | X | X | X |
| IFNAR2 | X | X | X | X | X | X | X | X | X | X | X | X | X | X |
| COL4A1 | X | X | X | X | X | X | X | X | X | X | X | X | X | X |
| FBLN2 | X | X | X | X | X | X | X | X | X | X | X | X | X | X |
| FBN2 | X | X | X | X | X | X | X | X | X | X | X | X | X | X |
| FCN1 | X | X | X | X | X | X | X | X | X | X | X | X | X | X |
| HS3ST4 | X | X | X | X | X | X | X | X | X | X | X | X | X | X |
| IGLC1 | X | X | X | X | X | X | X | X | X | X | X | X | X | X |
| IL12RB1 | X | X | X | X | X | X | X | X | X | X | X | X | X | X |
| ITGA4 | X | X | X | X | X | X | X | X | X | X | X | X | X | X |
| ITGAX | X | X | X | X | X | X | X | X | X | X | X | X | X | X |
| MASP1 | X | X | X | X | X | X | X | X | X | X | X | X | X | X |
| MASP2 | X | X | X | X | X | X | X | X | X | X | X | X | X | X |
| MYOC | X | X | X | X | X | X | X | X | X | X | X | X | X | X |
| PPID | X | X | X | X | X | X | X | X | X | X | X | X | X | X |
| PTPRC | X | X | X | X | X | X | X | X | X | X | X | X | X | X |
| SLC2A2 | X | X | X | X | X | X | X | X | X | X | X | X | X | X |
| SPOCK | X | X | X | X | X | X | X | X | X | X | X | X | X | X |
| TGFBR2 | X | X | X | X | X | X | X | X | X | X | X | X | X | X |
| C3 | X | X | X | X | X | X | X | X | X | X | X | X | X | X |
| C7 | X | X | X | X | X | X | X | X | X | X | X | X | X | X |

| | COL9A1 | FGFR2 | HABP2 | EMID2 | COL6A | IFNAR | COL4A | FBLN2 | FBN2 | FCN1 | HS3ST | IGLC1 | IL12RB |
|---|---|---|---|---|---|---|---|---|---|---|---|---|---|
| ADAM12 | X | X | X | X | X | X | X | X | X | X | X | X | X |
| ADAM19 | X | X | X | X | X | X | X | X | X | X | X | X | X |
| APBA2 | X | X | X | X | X | X | X | X | X | X | X | X | X |
| APOB | X | X | X | X | X | X | X | X | X | X | X | X | X |
| BMP7 | X | X | X | X | X | X | X | X | X | X | X | X | X |
| C1NH | X | X | X | X | X | X | X | X | X | X | X | X | X |
| C1Qa | X | X | X | X | X | X | X | X | X | X | X | X | X |
| C1RL | X | X | X | X | X | X | X | X | X | X | X | X | X |
| C4BPA | X | X | X | X | X | X | X | X | X | X | X | X | X |
| C5 | X | X | X | X | X | X | X | X | X | X | X | X | X |
| C8A | X | X | X | X | X | X | X | X | X | X | X | X | X |
| C9 | X | X | X | X | X | X | X | X | X | X | X | X | X |
| CCL28 | X | X | X | X | X | X | X | X | X | X | X | X | X |
| CLU | X | X | X | X | X | X | X | X | X | X | X | X | X |
| COL9A1 | | X | X | X | X | X | X | X | X | X | X | X | X |
| FGFR2 | X | | X | X | X | X | X | X | X | X | X | X | X |
| HABP2 | X | X | | X | X | X | X | X | X | X | X | X | X |
| EMID2 | X | X | X | | X | X | X | X | X | X | X | X | X |
| COL6A3 | X | X | X | X | | X | X | X | X | X | X | X | X |
| IFNAR2 | X | X | X | X | X | | X | X | X | X | X | X | X |
| COL4A1 | X | X | X | X | X | X | | X | X | X | X | X | X |
| FBLN2 | X | X | X | X | X | X | X | | X | X | X | X | X |
| FBN2 | X | X | X | X | X | X | X | X | | X | X | X | X |
| FCN1 | X | X | X | X | X | X | X | X | X | | X | X | X |
| HS3ST4 | X | X | X | X | X | X | X | X | X | X | | X | X |
| IGLC1 | X | X | X | X | X | X | X | X | X | X | X | | X |
| IL12RB1 | X | X | X | X | X | X | X | X | X | X | X | X | |
| ITGA4 | X | X | X | X | X | X | X | X | X | X | X | X | X |
| ITGAX | X | X | X | X | X | X | X | X | X | X | X | X | X |
| MASP1 | X | X | X | X | X | X | X | X | X | X | X | X | X |
| MASP2 | X | X | X | X | X | X | X | X | X | X | X | X | X |
| MYOC | X | X | X | X | X | X | X | X | X | X | X | X | X |
| PPID | X | X | X | X | X | X | X | X | X | X | X | X | X |
| PTPRC | X | X | X | X | X | X | X | X | X | X | X | X | X |
| SLC2A2 | X | X | X | X | X | X | X | X | X | X | X | X | X |
| SPOCK | X | X | X | X | X | X | X | X | X | X | X | X | X |
| TGFBR2 | X | X | X | X | X | X | X | X | X | X | X | X | X |
| C3 | X | X | X | X | X | X | X | X | X | X | X | X | X |
| C7 | X | X | X | X | X | X | X | X | X | X | X | X | X |

| | ITGA4 | ITGAX | MASP1 | MASP2 | MYOC | PPID | PTPRC | SLC2A | SPOCK | TGFBR | C3 | C7 |
|---|---|---|---|---|---|---|---|---|---|---|---|---|
| ADAM12 | X | X | X | X | X | X | X | X | X | X | X | X |
| ADAM19 | X | X | X | X | X | X | X | X | X | X | X | X |
| APBA2 | X | X | X | X | X | X | X | X | X | X | X | X |
| APOB | X | X | X | X | X | X | X | X | X | X | X | X |
| BMP7 | X | X | X | X | X | X | X | X | X | X | X | X |
| C1NH | X | X | X | X | X | X | X | X | X | X | X | X |
| C1Qa | X | X | X | X | X | X | X | X | X | X | X | X |
| C1RL | X | X | X | X | X | X | X | X | X | X | X | X |
| C4BPA | X | X | X | X | X | X | X | X | X | X | X | X |
| C5 | X | X | X | X | X | X | X | X | X | X | X | X |
| C8A | X | X | X | X | X | X | X | X | X | X | X | X |
| C9 | X | X | X | X | X | X | X | X | X | X | X | X |
| CCL28 | X | X | X | X | X | X | X | X | X | X | X | X |
| CLU | X | X | X | X | X | X | X | X | X | X | X | X |
| COL9A1 | X | X | X | X | X | X | X | X | X | X | X | X |
| FGFR2 | X | X | X | X | X | X | X | X | X | X | X | X |
| HABP2 | X | X | X | X | X | X | X | X | X | X | X | X |
| EMID2 | X | X | X | X | X | X | X | X | X | X | X | X |
| COL6A3 | X | X | X | X | X | X | X | X | X | X | X | X |
| IFNAR2 | X | X | X | X | X | X | X | X | X | X | X | X |
| COL4A1 | X | X | X | X | X | X | X | X | X | X | X | X |
| FBLN2 | X | X | X | X | X | X | X | X | X | X | X | X |
| FBN2 | X | X | X | X | X | X | X | X | X | X | X | X |
| FCN1 | X | X | X | X | X | X | X | X | X | X | X | X |
| HS3ST4 | X | X | X | X | X | X | X | X | X | X | X | X |
| IGLC1 | X | X | X | X | X | X | X | X | X | X | X | X |
| IL12RB1 | X | X | X | X | X | X | X | X | X | X | X | X |
| ITGA4 | | X | X | X | X | X | X | X | X | X | X | X |
| ITGAX | X | | X | X | X | X | X | X | X | X | X | X |
| MASP1 | X | X | | X | X | X | X | X | X | X | X | X |
| MASP2 | X | X | X | | X | X | X | X | X | X | X | X |
| MYOC | X | X | X | X | | X | X | X | X | X | X | X |
| PPID | X | X | X | X | X | | X | X | X | X | X | X |
| PTPRC | X | X | X | X | X | X | | X | X | X | X | X |
| SLC2A2 | X | X | X | X | X | X | X | | X | X | X | X |
| SPOCK | X | X | X | X | X | X | X | X | | X | X | X |
| TGFBR2 | X | X | X | X | X | X | X | X | X | | X | X |
| C3 | X | X | X | X | X | X | X | X | X | X | | X |
| C7 | X | X | X | X | X | X | X | X | X | X | X | |

In a further embodiment, the determination of an individual's genetic profile can include screening for a deletion or a heterozygous deletion within the RCA locus that is associated with AMD risk or protection. Exemplary deletions that are associated with AMD protection include deletion of FHR3 and FHR1 genes. The deletion may encompass one gene, multiple genes, a portion of a gene, or an intergenic region, for example. If the deletion impacts the size, conformation, expression or stability of an encoded protein, the deletion can be detected by assaying the protein, or by querying the nucleic acid sequence of the genome or transcriptome of the individual.

Further, determining an individual's genetic profile may include determining an individual's genotype or haplotype to determine if the individual is at an increased or decreased risk of developing AMD. In one embodiment, an individual's genetic profile may comprise SNPs that are in linkage disequilibrium with other SNPs associated with AMD that define a haplotype (i.e., a set of polymorphisms in the RCA locus) associated with risk or protection of AMD. In another embodiment, a genetic profile may include multiple haplotypes present in the genome or a combination of haplotypes and polymorphisms, such as single nucleotide polymorphisms, in the genome, e.g., a haplotype in the RCA locus and a haplotype or at least one SNP on chromosome 10.

Further studies of the identity of the various SNPs and other genetic characteristics disclosed herein with additional cohorts, and clinical experience with the practice of this invention on patient populations, will permit ever more precise assessment of AMD risk bases on emergent SNP patterns. This work will result in refinement of which particular set of SNPs are characteristic of a genetic profile which is, for example, indicative of an urgent need for intervention, or indicative that the early stages of AMD observed in a individual is unlikely to progress to more serious disease, or is likely to progress rapidly to the wet form of the disease, or that the presenting individual is not at significant risk of developing AMD, or that a particular AMD therapy is most likely to be successful with this individual and another therapeutic alternative less likely to be productive. Thus, it is anticipated that the practice of the invention disclosed herein, especially when combined with the practice of risk assessment using other known risk-indicative and protection-indicative SNPs, will permit disease management and avoidance with increasing precision.

A single nucleotide polymorphism comprised within a genetic profile for AMD as described herein may be detected directly or indirectly. Direct detection refers to determining the presence or absence of a specific SNP identified in the genetic profile using a suitable nucleic acid, such as an oligonucleotide in the form of a probe or primer as described below. Alternatively, direct detection can include querying a pre-produced database comprising all or part of the individual's genome for a specific SNP in the genetic profile. Other direct methods are known to those skilled in the art. Indirect detection refers to determining the presence or absence of a specific SNP identified in the genetic profile by detecting a surrogate or proxy SNP that is in linkage disequilibrium with the SNP in the individual's genetic profile. Detection of a proxy SNP is indicative of a SNP of interest and is increasingly informative to the extent that the SNPs are in linkage disequilibrium, e.g., at least 50%, 60%, 70%, 80%, 90%, 95%, 98%, or about 100% LD. Another indirect method involves detecting allelic variants of proteins accessible in a sample from an individual that are consequent of a risk-associated or protection-associated allele in DNA that alters a codon.

It is also understood that a genetic profile as described herein may comprise one or more nucleotide polymorphism(s) that are in linkage disequilibrium with a polymorphism that is causative of disease. In this case, the SNP in the genetic profile is a surrogate SNP for the causative polymorphism.

Genetically linked SNPs, including surrogate or proxy SNPs, can be identified by methods known in the art. Non-random associations between polymorphisms (including single nucleotide polymorphisms, or SNPs) at two or more loci are measured by the degree of linkage disequilibrium (LD). The degree of linkage disequilibrium is influenced by a number of factors including genetic linkage, the rate of recombination, the rate of mutation, random drift, non-random mating and population structure. Moreover, loci that are in LD do not have to be located on the same chromosome, although most typically they occur as clusters of adjacent variations within a restricted segment of DNA. Polymorphisms that are in complete or close LD with a particular disease-associated SNP are also useful for screening, diagnosis, and the like.

SNPs in LD with each other can be identified using methods known in the art and SNP databases (e.g., the Perlegen database, at http://genome.perlegen.com/browser/download.html and others). For illustration, SNPs in linkage disequilibrium (LD) with the CFH SNP rs800292 were identified using the Perlegen database. This database groups SNPs into LD bins such that all SNPs in the bin are highly correlated to each other. For example, AMD-associated SNP rs800292 was identified in the Perlegen database under the identifier ‘afd0678310’. A LD bin (European LD bin #1003371; see table below) was then identified that contained linked SNPs—including afd1152252, afd4609785, afd4270948, afd0678315, afd0678311, and afd0678310—and annotations.

| SNP ID | | | | | | Allele Frequency |
|---|---|---|---|---|---|---|
| Perlegen | | SNP Position | | | | European |
| ‘afd’ ID* | ‘ss’ ID | Chromosome | Accession | Position | Alleles | American |
| afd1152252 | ss23875287 | 1 | NC_000001.5 | 193872580 | A/G | 0.21 |
| afd4609785 | ss23849009 | 1 | NC_000001.5 | 193903455 | G/A | 0.79 |
| afd4270948 | ss23849019 | 1 | NC_000001.5 | 193905168 | T/C | 0.79 |
| afd0678315 | ss23857746 | 1 | NC_000001.5 | 193923365 | G/A | 0.79 |
| afd0678311 | ss23857767 | 1 | NC_000001.5 | 193930331 | C/T | 0.79 |
| afd0678310 | ss23857774 | 1 | NC_000001.5 | 193930492 | G/A | 0.79 |

*Perlegen AFD identification numbers can be converted into conventional SNP database identifiers (in this case, rs4657825, rs576258, rs481595, rs529825, rs551397, and rs800292) using the NCBI database (http://www.ncbi.nlm.nih.gov/sites/entrez?db=snp&cmd=search&term=).

Also, for illustration, SNPs in linkage disequilibrium (LD) with the PTPCR SNP rs1932433 were identified using the Perlegen database, which groups SNPs into LD bins such that all SNPs in the bin are highly correlated to each other. For example, AMD-associated SNP rs1932433 was identified in the Perlegen database under its ‘afd’ identifier. A LD bin (see table below) was then identified that contained linked SNPs—including afd3989407, afd3989410, afd1154319, afd1154321, afd1154322, afd4258456, afd4214530, and afd4284908—and annotations.

| PTPCR | | | | | | Allele |
|---|---|---|---|---|---|---|
| SNP ID | | | | | | Frequency |
| Perlegen | | SNP Position | | | | European |
| ‘afd’ ID* | ss ID | Chromosome | Accession | Position | Alleles | American |
| afd3989407 | ss23850038 | 1 | NC_000001.5 | 195976204 | C/T | 0.28 |
| afd3989410 | ss23850048 | 1 | NC_000001.5 | 195983803 | G/C | 0.28 |
| afd1154319 | ss23870320 | 1 | NC_000001.5 | 195988050 | C/T | 0.29 |
| afd1154321 | ss23870329 | 1 | NC_000001.5 | 195989546 | T/C | 0.29 |
| afd1154322 | ss23870335 | 1 | NC_000001.5 | 195990918 | C/T | 0.29 |
| afd4258456 | ss23870351 | 1 | NC_000001.5 | 195995474 | A/C | 0.29 |
| afd4214530 | ss23870386 | 1 | NC_000001.5 | 196004761 | C/T | 0.29 |
| afd4284908 | ss23171843 | 1 | NC_000001.5 | 196018852 | A/G | 0.26 |

*Perlegen AFD identification numbers can be converted into conventional SNP database identifiers (in this case, rs6696003, rs4483440, rs12120762, rs1932433, rs4915155, rs7555443, rs4478839, and rs4915319) using the NCBI database (http://www.ncbi.nlm.nih.gov/sites/entrez?db=snp&cmd=search&term=).

The frequencies of these alleles in disease versus control populations may be determined using the methods described herein.

As a second example, the LD tables computed by HapMap were downloaded (http://ftp.hapmap.org/ld_data/latest/). Unlike the Perlegen database, the HapMap tables use ‘rs’ SNP identifiers directly. All SNPs with an R² value greater than 0.80 when compared to rs800292 were extracted from the database in this illustration. Due to the alternate threshold used to compare SNPs and the greater SNP coverage of the HapMap data, more SNPs were identified using the HapMap data than the Perlegen data.

| | SNP #2 | | | | | | |
|---|---|---|---|---|---|---|---|
| SNP 1 Location | Location | Population | SNP #1 ID | SNP #2 ID | D′ | R² | LOD |
| 194846662 | 194908856 | CEU | rs10801551 | rs800292 | 1 | 0.84 | 19.31 |
| 194850944 | 194908856 | CEU | rs4657825 | rs800292 | 1 | 0.9 | 21.22 |
| 194851091 | 194908856 | CEU | rs12061508 | rs800292 | 1 | 0.83 | 18.15 |
| 194886125 | 194908856 | CEU | rs505102 | rs800292 | 1 | 0.95 | 23.04 |
| 194899093 | 194908856 | CEU | rs6680396 | rs800292 | 1 | 0.84 | 19.61 |
| 194901729 | 194908856 | CEU | rs529825 | rs800292 | 1 | 0.95 | 23.04 |
| 194908856 | 194928161 | CEU | rs800292 | rs12124794 | 1 | 0.84 | 18.81 |
| 194908856 | 194947437 | CEU | rs800292 | rs1831281 | 1 | 0.84 | 19.61 |
| 194908856 | 194969148 | CEU | rs800292 | rs2284664 | 1 | 0.84 | 19.61 |
| 194908856 | 194981223 | CEU | rs800292 | rs10801560 | 1 | 0.84 | 19.61 |
| 194908856 | 194981293 | CEU | rs800292 | rs10801561 | 1 | 0.84 | 19.61 |
| 194908856 | 195089923 | CEU | rs800292 | rs10922144 | 1 | 0.84 | 19.61 |

As indicated above, publicly available databases such as the HapMap database (http://ftp.hapmap.org/ld_data/latest/) and Haploview (Barrett, J. C. et al., Bioinformatics 21, 263 (2005)) may be used to calculate linkage disequilibiurm between two SNPs. The frequency of identified alleles in disease versus control populations may be determined using the methods described herein. Statistical analyses may be employed to determine the significance of a non-random association between the two SNPs (e.g., Hardy-Weinberg Equilibrium, Genotype likelihood ratio (genotype p value), Chi Square analysis, Fishers Exact test). A statistically significant non-random association between the two SNPs indicates that they are in linkage disequilibrium and that one SNP can serve as a proxy for the second SNP.

The screening step to determine an individual's genetic profile may be conducted by inspecting a data set indicative of genetic characteristics previously derived from analysis of the individual's genome. A data set indicative of an individual's genetic characteristics may include a complete or partial sequence of the individual's genomic DNA, or a SNP map. Inspection of the data set including all or part of the individual's genome may optimally be performed by computer inspection. Screening may further comprise the step of producing a report identifying the individual and the identity of alleles at the site of at least one or more polymorphisms shown in Table I or II and/or proxy SNPs.

Alternatively, the screening step to determine an individual's genetic profile comprises analyzing a nucleic acid (i.e., DNA or RNA) sample obtained from the individual. A sample can be from any source containing nucleic acids (e.g., DNA or RNA) including tissues such as hair, skin, blood, biopsies of the retina, kidney, or liver or other organs or tissues, or sources such as saliva, cheek scrapings, urine, amniotic fluid or CVS samples, and the like. Typically, genomic DNA is analyzed. Alternatively, RNA, cDNA, or protein can be analyzed. Methods for the purification or partial purification of nucleic acids or proteins from an individual's sample, and various protocols for analyzing samples for use in diagnostic assays are well known.

A polymorphism such as a SNP can be conveniently detected using suitable nucleic acids, such as oligonucleotides in the form of primers or probes. Accordingly, the invention not only provides novel SNPs and/or novel combinations of SNPs that are useful in assessing risk for a complement-related disease, but also nucleic acids such as oligonucleotides useful to detect them. A useful oligonucleotide for instance comprises a sequence that hybridizes under stringent hybridization conditions to at least one polymorphism identified herein. Where appropriate, at least one oligonucleotide comprises a sequence that is fully complementary to a nucleic acid sequence comprising at least one polymorphism identified herein. Such oligonucleotide(s) can be used to detect the presence of the corresponding polymorphism, for example by hybridizing to the polymorphism under stringent hybridizing conditions, or by acting as an extension primer in either an amplification reaction such as PCR or a sequencing reaction, wherein the corresponding polymorphism is detected either by amplification or sequencing. Suitable detection methods are described below.

An individual's genotype can be determined using any method capable of identifying nucleotide variation, for instance at single nucleotide polymorphic sites. The particular method used is not a critical aspect of the invention. Although considerations of performance, cost, and convenience will make particular methods more desirable than others, it will be clear that any method that can detect one or more polymorphisms of interest can be used to practice the invention. A number of suitable methods are described below.

### 1) Nucleic Acid Analysis

### General

Polymorphisms can be identified through the analysis of the nucleic acid sequence present at one or more of the polymorphic sites. A number of such methods are known in the art. Some such methods can involve hybridization, for instance with probes (probe-based methods). Other methods can involve amplification of nucleic acid (amplification-based methods). Still other methods can include both hybridization and amplification, or neither.

### a) Amplification-Based Methods

### Preamplification Followed by Sequence Analysis:

Where useful, an amplification product that encompasses a locus of interest can be generated from a nucleic acid sample. The specific polymorphism present at the locus is then determined by further analysis of the amplification product, for instance by methods described below. Allele-independent amplification can be achieved using primers which hybridize to conserved regions of the genes. The genes contain many invariant or monomorphic regions and suitable allele-independent primers can be selected routinely.

Upon generation of an amplified product, polymorphisms of interest can be identified by DNA sequencing methods, such as the chain termination method (Sanger et al., 1977, Proc. Natl. Acad. Sci., 74:5463-5467) or PCR-based sequencing. Other useful analytical techniques that can detect the presence of a polymorphism in the amplified product include single-strand conformation polymorphism (SSCP) analysis, denaturing gradient gel electropohoresis (DGGE) analysis, and/or denaturing high performance liquid chromatography (DHPLC) analysis. In such techniques, different alleles can be identified based on sequence- and structure-dependent electrophoretic migration of single stranded PCR products. Amplified PCR products can be generated according to standard protocols, and heated or otherwise denatured to form single stranded products, which may refold or form secondary structures that are partially dependent on base sequence. An alternative method, referred to herein as a kinetic-PCR method, in which the generation of amplified nucleic acid is detected by monitoring the increase in the total amount of double-stranded DNA in the reaction mixture, is described in Higuchi et al., 1992, Bio/Technology, 10:413-417, incorporated herein by reference.

### Allele-Specific Amplification:

Alleles can also be identified using amplification-based methods. Various nucleic acid amplification methods known in the art can be used in to detect nucleotide changes in a target nucleic acid. Alleles can also be identified using allele-specific amplification or primer extension methods, in which amplification or extension primers and/or conditions are selected that generate a product only if a polymorphism of interest is present.

### Amplification Technologies

A preferred method is the polymerase chain reaction (PCR), which is now well known in the art, and described in U.S. Pat. Nos. 4,683,195; 4,683,202; and 4,965,188; each incorporated herein by reference. Other suitable amplification methods include the ligase chain reaction (Wu and Wallace, 1988, Genomics 4:560-569); the strand displacement assay (Walker et al., 1992, Proc. Natl. Acad. Sci. USA 89:392-396, Walker et al. 1992, Nucleic Acids Res. 20:1691-1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., 1989, Proc. Natl. Acad. Sci. USA, 86:1173-1177); and self-sustained sequence replication (3SR) (Guatelli et al., 1990, Proc. Natl. Acad. Sci. USA, 87:1874-1878 and WO 92/08800); each incorporated herein by reference. Alternatively, methods that amplify the probe to detectable levels can be used, such as QB-replicase amplification (Kramer et al., 1989, Nature, 339:401-402, and Lomeli et al., 1989, Clin. Chem., 35:1826-1831, both of which are incorporated herein by reference). A review of known amplification methods is provided in Abramson et al., 1993, Current Opinion in Biotechnology, 4:41-47, incorporated herein by reference.

Amplification of mRNA

Genotyping also can also be carried out by detecting and analyzing mRNA under conditions when both maternal and paternal chromosomes are transcribed. Amplification of RNA can be carried out by first reverse-transcribing the target RNA using, for example, a viral reverse transcriptase, and then amplifying the resulting cDNA, or using a combined high-temperature reverse-transcription-polymerase chain reaction (RT-PCR), as described in U.S. Pat. Nos. 5,310,652; 5,322,770; 5,561,058; 5,641,864; and 5,693,517; each incorporated herein by reference (see also Myers and Sigua, 1995, in PCR Strategies, supra, chapter 5).

### Selection of Allele-Specific Primers

The design of an allele-specific primer can utilize the inhibitory effect of a terminal primer mismatch on the ability of a DNA polymerase to extend the primer. To detect an allele sequence using an allele-specific amplification or extension-based method, a primer complementary to the genes of interest is chosen such that the nucleotide hybridizes at or near the polymorphic position. For instance, the primer can be designed to exactly match the polymorphism at the 3′ terminus such that the primer can only be extended efficiently under stringent hybridization conditions in the presence of nucleic acid that contains the polymorphism. Allele-specific amplification- or extension-based methods are described in, for example, U.S. Pat. Nos. 5,137,806; 5,595,890; 5,639,611; and U.S. Pat. No. 4,851,331, each incorporated herein by reference.

### Analysis of Heterozygous Samples

If so desired, allele-specific amplification can be used to amplify a region encompassing multiple polymorphic sites from only one of the two alleles in a heterozygous sample.

### b) Probe-Based Methods:

### General

Alleles can be also identified using probe-based methods, which rely on the difference in stability of hybridization duplexes formed between a probe and its corresponding target sequence comprising an allele. For example, differential probes can be designed such that under sufficiently stringent hybridization conditions, stable duplexes are formed only between the probe and its target allele sequence, but not between the probe and other allele sequences.

### Probe Design

A suitable probe for instance contains a hybridizing region that is either substantially complementary or exactly complementary to a target region of a polymorphism described herein or their complement, wherein the target region encompasses the polymorphic site. The probe is typically exactly complementary to one of the two allele sequences at the polymorphic site. Suitable probes and/or hybridization conditions, which depend on the exact size and sequence of the probe, can be selected using the guidance provided herein and well known in the art. The use of oligonucleotide probes to detect nucleotide variations including single base pair differences in sequence is described in, for example, Conner et al., 1983, Proc. Natl. Acad. Sci. USA, 80:278-282, and U.S. Pat. Nos. 5,468,613 and 5,604,099, each incorporated herein by reference.

### Pre-Amplification Before Probe Hybridization

In an embodiment, at least one nucleic acid sequence encompassing one or more polymorphic sites of interest are amplified or extended, and the amplified or extended product is hybridized to one or more probes under sufficiently stringent hybridization conditions. The alleles present are inferred from the pattern of binding of the probes to the amplified target sequences.

### Some Known Probe-Based Genotyping Assays

Probe-based genotyping can be carried out using a “TaqMan” or “5′-nuclease assay,” as described in U.S. Pat. Nos. 5,210,015; 5,487,972; and 5,804,375; and Holland et al., 1988, Proc. Natl. Acad. Sci. USA, 88:7276-7280, each incorporated herein by reference. Examples of other techniques that can be used for SNP genotyping include, but are not limited to, Amplifluor, Dye Binding-Intercalation, Fluorescence Resonance Energy Transfer (FRET), Hybridization Signal Amplification Method (HSAM), HYB Probes, Invader/Cleavase Technology (Invader/CFLP), Molecular Beacons, Origen, DNA-Based Ramification Amplification (RAM), Rolling circle amplification (RCA), Scorpions, Strand displacement amplification (SDA), oligonucleotide ligation (Nickerson et al., Proc. Natl. Acad. Sci. USA, 87: 8923-8927) and/or enzymatic cleavage. Popular high-throughput SNP-detection methods also include template-directed dye-terminator incorporation (TDI) assay (Chen and Kwok, 1997, Nucleic Acids Res. 25: 347-353), the 5′-nuclease allele-specific hybridization TaqMan assay (Livak et al. 1995, Nature Genet. 9: 341-342), and the recently described allele-specific molecular beacon assay (Tyagi et al. 1998, Nature Biotech. 16: 49-53).

### Assay Formats

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference. In some embodiments multiple assays are conducted using a microfluidic format. See, e.g., Unger et al., 2000, Science 288:113-6.

### Nucleic Acids Containing Polymorphisms of Interest

The invention also provides isolated nucleic acid molecules, e.g., oligonucleotides, probes and primers, comprising a portion of the genes, their complements, or variants thereof as identified herein. Preferably the variant comprises or flanks at least one of the polymorphic sites identified herein, for example variants associated with AMD.

Nucleic acids such as primers or probes can be labeled to facilitate detection. Oligonucleotides can be labeled by incorporating a label detectable by spectroscopic, photochemical, biochemical, immunochemical, radiological, radiochemical or chemical means. Useful labels include ³²P, fluorescent dyes, electron-dense reagents, enzymes, biotin, or haptens and proteins for which antisera or monoclonal antibodies are available.

### 2) Protein-Based or Phenotypic Detection of Polymorphism:

Where polymorphisms are associated with a particular phenotype, then individuals that contain the polymorphism can be identified by checking for the associated phenotype. For example, where a polymorphism causes an alteration in the structure, sequence, expression and/or amount of a protein or gene product, and/or size of a protein or gene product, the polymorphism can be detected by protein-based assay methods.

### Techniques for Protein Analysis

Protein-based assay methods include electrophoresis (including capillary electrophoresis and one- and two-dimensional electrophoresis), chromatographic methods such as high performance liquid chromatography (HPLC), thin layer chromatography (TLC), hyperdiffusion chromatography, and mass spectrometry.

### Antibodies

Where the structure and/or sequence of a protein is changed by a polymorphism of interest, one or more antibodies that selectively bind to the altered form of the protein can be used. Such antibodies can be generated and employed in detection assays such as fluid or gel precipitin reactions, immunodiffusion (single or double), immunoelectrophoresis, radioimmunoassay (RIA), enzyme-linked immunosorbent assays (ELISAs), immunofluorescent assays, Western blotting and others.

### 3) Kits

In certain embodiments, one or more oligonucleotides of the invention are provided in a kit or on an array useful for detecting the presence of a predisposing or a protective polymorphism in a nucleic acid sample of an individual whose risk for a complement-related disease such as AMD is being assessed. A useful kit can contain oligonucleotide specific for particular alleles of interest as well as instructions for their use to determine risk for a complement-related disease such as AMD. In some cases, the oligonucleotides may be in a form suitable for use as a probe, for example fixed to an appropriate support membrane. In other cases, the oligonucleotides can be intended for use as amplification primers for amplifying regions of the loci encompassing the polymorphic sites, as such primers are useful in the preferred embodiment of the invention. Alternatively, useful kits can contain a set of primers comprising an allele-specific primer for the specific amplification of alleles. As yet another alternative, a useful kit can contain antibodies to a protein that is altered in expression levels, structure and/or sequence when a polymorphism of interest is present within an individual. Other optional components of the kits include additional reagents used in the genotyping methods as described herein. For example, a kit additionally can contain amplification or sequencing primers which can, but need not, be sequence-specific, enzymes, substrate nucleotides, reagents for labeling and/or detecting nucleic acid and/or appropriate buffers for amplification or hybridization reactions.

### 4) Arrays

The present invention also relates to an array, a support with immobilized oligonucleotides useful for practicing the present method. A useful array can contain oligonucleotide probes specific for polymorphisms identified herein. The oligonucleotides can be immobilized on a substrate, e.g., a membrane or glass. The oligonucleotides can, but need not, be labeled. The array can comprise one or more oligonucleotides used to detect the presence of one or more SNPs provided herein. In some embodiments, the array can be a micro-array.

The array can include primers or probes to determine assay the presence or absence of at least two of the SNPs listed in Tables I and/or II, sometimes at least three, at least four, at least five or at least six of the SNPs. In one embodiment, the array comprises probes or primers for detection of fewer than about 1000 different SNPs, often fewer than about 100 different SNPs, and sometimes fewer than about 50 different SNPs.

### VI. Therapeutic Nucleic Acids Encoding Polypeptides

In certain embodiments, the invention provides isolated and/or recombinant nucleic acids corresponding to any one of the genes shown in Table I or II encoding polypeptides, including functional variants selected from the group consisting of ADAM12, ADAM19, APBA2, APOB, BMP7, C1NH, C1Qa, C1RL, C4BPA, C5, C8A, C9, CCL28, CLU, COL9A1, FGFR2, HABP2, EMID2, COL6A3, IFNAR2, COL4A1, FBLN2, FBN2, FCN1, HS3ST4, IGLC1, IL12RB1, ITGA4, ITGAX, MASP1, MASP2, MYOC, PPID, PTPRC, SLC2A2, SPOCK, TGFBR2, C3, and C7. In certain embodiments the functional variants include dominant negative variants. One skilled in the art will understand dominant negative variants to be polypeptides that compete with the wildtype polypeptides for a certain function. The utility of dominant negative variants and concepts of generating dominant negative variants are well known in the art and have been applied in many context for a long time (see, for example, Mendenhall M, PNAS, 85:4426-4430 (1988); Haruki N, Cancer Res. 65:3555-3561 (2005)) and some dominant negative proteins are produced commercially (for example, by Cytoskeleton).

The subject nucleic acids may be single-stranded or double stranded. Such nucleic acids may be DNA or RNA molecules. These nucleic acids may be used, for example, in methods for making a polypeptide selected from the group consisting of ADAM12, ADAM19, APBA2, APOB, BMP7, C1NH, C1Qa, C1RL, C4BPA, C5, C8A, C9, CCL28, CLU, COL9A1, FGFR2, HABP2, EMID2, COL6A3, IFNAR2, COL4A1, FBLN2, FBN2, FCN1, HS3ST4, IGLC1, IL12RB1, ITGA4, ITGAX, MASP1, MASP2, MYOC, PPID, PTPRC, SLC2A2, SPOCK, TGFBR2, C3, and C7, or as direct therapeutic agents (e.g., in a gene therapy approach).

In certain embodiments, the invention provides isolated or recombinant nucleic acid sequences that are at least 80%, 85%, 90%, 95%, 97%, 98%, 99% or 100% identical to the sequences for any one of the genes shown in Table I or II. One of ordinary skill in the art will appreciate that nucleic acid sequences complementary to the sequences shown in Table V, and variants of the sequences shown in Table V are also within the scope of this invention. In further embodiments, the nucleic acid sequences of the invention can be isolated, recombinant, and/or fused with a heterologous nucleotide sequence, or in a DNA library.

In other embodiments, nucleic acids of the invention also include nucleic acids that hybridize under stringent conditions to the nucleotide sequence designated in the sequences in Table V, complement sequence of the sequences in Table V, or fragments thereof. As discussed above, one of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the invention provides nucleic acids which hybridize under low stringency conditions of 6×SSC at room temperature followed by a wash at 2×SSC at room temperature.

Isolated nucleic acids which differ from the wildtype nucleic acids for any of the genes shown in Tables I and/or II due to degeneracy in the genetic code are also within the scope of the invention. For example, a number of amino acids are designated by more than one triplet. Codons that specify the same amino acid, or synonyms (for example, CAU and CAC are synonyms for histidine) may result in “silent” variations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of thejtiucleotides) of the nucleic acids encoding a particular protein may exist among individuals of a given species due to natural allelic variation. Any and all such nucleotide variations and resulting amino acid polymorphisms are within the scope of this invention.

The nucleic acids and polypeptides of the invention may be produced using standard recombinant methods. For example, the recombinant nucleic acids of the invention may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the invention. The promoters may be either naturally occurring promoters, or hybrid promoters that combine elements of more than one promoter. An expression construct may be present in a cell on an episome, such as a plasmid, or the expression construct may be inserted in a chromosome. The expression vector may also contain a selectable marker gene to allow the selection of transformed host cells. Selectable marker genes are well known in the art and will vary with the host cell used. In certain embodiments of the invention, the subject nucleic acid is provided in an expression vector comprising a nucleotide sequence encoding polypeptide selected from the group consisting of ADAM12, ADAM19, APBA2, APOB, BMP7, C1Qa, C1RL, C4BPA, C5, C8A, CCL28, CLU, COL9A1, FGFR2, HABP2, EMID2, COL6A3, IFNAR2, COL4A1, FBLN2, FBN2, FCN1, HS3ST4, IGLC1, IL12RB1, ITGAX, MASP1, MASP2, MYOC, PPID, PTPRC, SLC2A2, SPOCK, TGFBR2, C3, C7, C9, C1NH, and ITGA4, and operably linked to at least one regulatory sequence. Regulatory sequences are art-recognized and are selected to direct expression of a selected polypeptide. Accordingly, the term “regulatory sequence” includes promoters, enhancers, termination sequences, preferred ribosome binding site sequences, preferred mRNA leader sequences, preferred protein processing sequences, preferred signal sequences for protein secretion, and other expression control elements. Examples of regulatory sequences are described in Goeddel; Gene Expression Technology: Methods in Enzymology, Academic Press, San Diego, Calif. (1990). For instance, any of a wide variety of expression control sequences that control the expression of a DNA sequence when operatively linked to it may be used in these vectors to express DNA sequences encoding a polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the invention can be produced by ligating the cloned gene, or a portion thereof, into a vector suitable for expression in either prokaryotic cells, eukaryotic cells (yeast, avian, insect or mammalian), or both. Expression vehicles for production of recombinant polypeptides include plasmids and other vectors. For instance, suitable vectors include plasmids of the types: pBR322— derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic, transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells.

Alternatively, derivatives of viruses such as the bovine papilloma virus (BPV-I), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning: A Laboratory Manual, Cold Spring Harbor Laboratory (2001). In some instances, it may be desirable to express the recombinant polypeptide by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In one embodiment, a vector will be designed for production of a selected polypeptide in CHO cells, such as a Pcmv-Script vector (Stratagene, La Jolla, Calif.), pcDNA4 vectors (Invitrogen, Carlsbad, Calif.) and pCI-neo vectors (Promega, Madison, Wise). In other embodiments, the vector is designed for production of a subject SRF, AP2 alpha, HTRA1 or CFH polypeptide in prokaryotic host cells (e.g., E. coli and B. subtilis), eukaryotic host cells such as, for example, yeast cells, insect cells, myeloma cells, fibroblast 3T3 cells, monkey kidney or COS cells, mink-lung epithelial cells, human foreskin fibroblast cells, human glioblastoma cells, and teratocarcinoma cells. Alternatively, the genes may be expressed in a cell-free system such as the rabbit reticulocyte lysate system.

As will be apparent, the subject gene constructs can be used to express the selected polypeptide in cells propagated in culture, e.g., to produce proteins, including fusion proteins or variant proteins, for purification. This invention also pertains to a host cell transfected with a recombinant gene including a coding sequence for one or more of the selected polypeptides. The host cell may be any prokaryotic or eukaryotic cell. For example, a selected polypeptide of the invention may be expressed in bacterial cells such as E. coli, insect cells (e.g., using a baculovirus expression system), yeast, or mammalian cells. Other suitable host cells are known to those skilled in the art.

Accordingly, the present invention further pertains to methods of producing a polypeptide selected from the group consisting of ADAM12, ADAM19, APBA2, APOB, BMP7, C1Qa, C1RL, C4BPA, C5, C8A, CCL28, CLU, COL9A1, FGFR2, HABP2, EMID2, COL6A3, IFNAR2, COL4A1, FBLN2, FBN2, FCN1, HS3ST4, IGLC1, IL12RB1, ITGAX, MASP1, MASP2, MYOC, PPID, PTPRC, SLC2A2, SPOCK, TGFBR2, C3, C7, C9, C1NH, and ITGA4. For example, a host cell transfected with an expression vector encoding a selected polypeptide can be cultured under appropriate conditions to allow expression of the selected polypeptide to occur. As such, the polypeptide may be secreted and isolated from a mixture of cells and medium containing the selected polypeptide. Alternatively, the polypeptide may be retained cytoplasmically or in a membrane fraction and the cells harvested, lysed and the protein isolated. A cell culture includes host cells, media and other byproducts. Suitable media for cell culture are well known in the art. The polypeptide can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the polypeptide. In a particular embodiment, the selected polypeptide is a fusion protein containing a domain which facilitates the purification of said polypeptide.

In another embodiment, a fusion gene coding for a purification leader sequence, such as a poly-(His)/enterokinase cleavage site sequence at the N-terminus of the desired portion of the recombinant polypeptide, can allow purification of the expressed fusion protein by affinity chromatography using a Ni²⁺ metal resin. The purification leader sequence can then be subsequently removed by treatment with enterokinase to provide the purified polypeptide (e.g., see Hochuli et al., (1987) J. Chromatography 411:177; and Janknecht et al., PNAS USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### VII. Other Therapeutic Modalities

### Antisense Polynucleotides

In certain embodiments, the invention provides polynucleotides that comprise an antisense sequence that acts through an antisense mechanism for inhibiting expression of any one of the genes listed in Table I or II. Antisense technologies have been widely utilized to regulate gene expression (Buskirk et al., Chem. Biol. 11, 1157-63 (2004); and Weiss et al., Cell MoI Life Sci 55, 334-58 (1999)). As used herein, “antisense” technology refers to administration or in situ generation of molecules or their derivatives which specifically hybridize (e.g., bind) under cellular conditions, with the target nucleic acid of interest (mRNA and/or genomic DNA) encoding one or more of the target proteins so as to inhibit expression of that protein, e.g., by inhibiting transcription and/or translation, such as by steric hinderance, altering splicing, or inducing cleavage or other enzymatic inactivation of the transcript. The binding may be by conventional base pair complementarity, or, for example, in the case of binding to DNA duplexes, through specific interactions in the major groove of the double helix. In general, “antisense” technology refers to the range of techniques generally employed in the art, and includes any therapy that relies on specific binding to nucleic acid sequences.

A polynucleotide that comprises an antisense sequence of the present invention can be delivered, for example, as a component of an expression plasmid which, when transcribed in the cell, produces a nucleic acid sequence that is complementary to at least a unique portion of the target nucleic acid. Alternatively, the polynucleotide that comprises an antisense sequence can be generated outside of the target cell, and which, when introduced into the target cell causes inhibition of expression by hybridizing with the target nucleic acid. Polynucleotides of the invention may be modified so that they are resistant to endogenous nucleases, e.g. exonucleases and/or endonucleases, and are therefore stable in vivo. Examples of nucleic acid molecules for use in polynucleotides of the invention are phosphoramidate, phosphothioate and methylphosphonate analogs of DNA (see also U.S. Pat. Nos. 5,176,996; 5,264,564; and 5,256,775). General approaches to constructing polynucleotides useful in antisense technology have been reviewed, for example, by van der Krol et al. (1988) Biotechniques 6:958-976; and Stein et al. (1988) Cancer Res 48:2659-2668.

Antisense approaches involve the design of polynucleotides (either DNA or RNA) that are complementary to a target nucleic acid encoding a risk-associated polymorphism of any one of the genes shown in Table I or II. The antisense polynucleotide may bind to an mRNA transcript and prevent translation of a protein of interest. Absolute complementarity, although preferred, is not required. In the case of double-stranded antisense polynucleotides, a single strand of the duplex DNA may thus be tested, or triplex formation may be assayed. The ability to hybridize will depend on both the degree of complementarity and the length of the antisense sequence. Generally, the longer the hybridizing nucleic acid, the more base mismatches with a target nucleic acid it may contain and still form a stable duplex (or triplex, as the case may be). One skilled in the art can ascertain a tolerable degree of mismatch by use of standard procedures to determine the melting point of the hybridized complex.

Antisense polynucleotides that are complementary to the 5′ end of an mRNA target, e.g., the 5′ untranslated sequence up to and including the AUG initiation codon, should work most efficiently at inhibiting translation of the mRNA. However, sequences complementary to the 3′ untranslated sequences of mRNAs have been shown to be effective at inhibiting translation of mRNAs as well (Wagner, R. 1994. Nature 372:333). Therefore, antisense polynucleotides complementary to either the 5′ or 3′ untranslated, non-coding regions of a variant gene shown in Table I or II could be used in an antisense approach to inhibit translation of a corresponding variant mRNA. Antisense polynucleotides complementary to the 5′ untranslated region of an mRNA should include the complement of the AUG start codon. Antisense polynucleotides complementary to mRNA coding regions are less efficient inhibitors of translation but could also be used in accordance with the invention. Whether designed to hybridize to the 5′, 3′, or coding region of mRNA, antisense polynucleotides should be at least six nucleotides in length, and are preferably less that about 100 and more preferably less than about 50, 25, 17 or 10 nucleotides in length.

Regardless of the choice of target sequence, it is preferred that in vitro studies are first performed to quantitate the ability of the antisense polynucleotide to inhibit expression of the selected gene. It is preferred that these studies utilize controls that distinguish between antisense gene inhibition and nonspecific biological effects of antisense polynucleotide. It is also preferred that these studies compare levels of the target KNA or protein with that of an internal control RNA or protein. Additionally, it is envisioned that results obtained using the antisense polynucleotide are compared with those obtained using a control antisense polynucleotide. It is preferred that the control antisense polynucleotide is of approximately the same length as the test antisense polynucleotide and that the nucleotide sequence of the control antisense polynucleotide differs from the antisense sequence of interest no more than is necessary to prevent specific hybridization to the target sequence.

Polynucleotides of the invention, including antisense polynucleotides, can be DNA or RNA or chimeric mixtures or derivatives or modified versions thereof, single-stranded or double-stranded. Polynucleotides of the invention can be modified at the base moiety, sugar moiety, or phosphate backbone, for example, to improve stability of the molecule, hybridization, etc. Polynucleotides of the invention may include other appended groups such as peptides (e.g., for targeting host cell receptors), or agents facilitating transport across the cell membrane (see, e.g., Letsinger et al., 1989, Proc Natl Acad. Sci. USA 86:6553-6556; Lemaitre et al., 1987, Proc Natl Acad Sci USA 84:648-652; PCT Publication No. W088/09810, published Dec. 15, 1988) or the blood-brain barrier (see, e.g., PCT Publication No. W089/10134, published Apr. 25, 1988), hybridization-triggered cleavage agents. (See, e.g., Krol et al., 1988, BioTechniques 6:958-976) or intercalating agents. (See, e.g., Zon, Pharm. Res. 5:539-549 (1988)). To this end, a polynucleotide of the invention may be conjugated to another molecule, e.g., a peptide, hybridization triggered cross-linking agent, transport agent, hybridization-triggered cleavage agent, etc.

Polynucleotides of the invention, including antisense polynucleotides, may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil; beta-D-mannosylqueosine, 5-methoxycarboxymethyruracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N2-carboxypropyl)uracil, and 2,6-diaminopurine.

Polynucleotides of the invention may also comprise at least one modified sugar moiety selected from the group including but not limited to arabinose, 2-fiuoroarabinose, xylulose, and hexose.

A polynucleotide of the invention can also contain a neutral peptide-like backbone. Such molecules are termed peptide nucleic acid (PNA)-oligomers and are described, e.g., in Perry-O'Keefe et al. (1996) Proc Natl Acad Sci USA 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, a polynucleotide of the invention comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, polynucleotides of the invention, including antisense polynucleotides are anomeric oligonucleotides. An anomeric oligonucleotide forms specific double-stranded hybrids with complementary RNA in which, contrary to the usual units, the strands run parallel to each other (Gautier et al., 1987, Nucl AcidsRes. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Polynucleotides of the invention, including antisense polynucleotides, may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. Nucl. Acids Res. 16:3209 (1988)), methylphosphonate oligonucleotides can be prepared by use of controlled pore glass polymer supports (Sarin et al., Proc Natl Acad Sci USA 85:7448-7451 (1988)).

While antisense sequences complementary to the coding region of an mRNA sequence can be used, those complementary to the transcribed untranslated region and to the region comprising the initiating methionine are most preferred.

Antisense polynucleotides can be delivered to cells that express target genes in vivo. A number of methods have been developed for delivering nucleic acids into cells; e.g., they can be injected directly into the tissue site, or modified nucleic acids, designed to target the desired cells (e.g., antisense polynucleotides linked to peptides or antibodies that specifically bind receptors or antigens expressed on the target cell surface) can be administered systematically.

However, it may be difficult to achieve intracellular concentrations of the antisense polynucleotides sufficient to attenuate the activity of a selected gene or mRNA in certain instances. Therefore, another approach utilizes a recombinant DNA construct in which the antisense polynucleotide is placed under the control of a strong pol III or pol II promoter. The use of such a construct to transfect target cells in the patient will result in the transcription of sufficient amounts of antisense polynucleotides that will form complementary base pairs with the selected gene or mRNA and thereby attenuate the activity of said protein. For example, a vector can be introduced in vivo such that it is taken up by a cell and directs the transcription of an antisense polynucleotide that targets a selected gene or mRNA. Such a vector can remain episomal or become chromosomally integrated, as long as it can be transcribed to produce the desired antisense polynucleotide. Such vectors can be constructed by recombinant DNA technology methods standard in the art. Vectors can be plasmid, viral, or others known in the art, used for replication and expression in mammalian cells. A promoter may be operably linked to the sequence encoding the antisense polynucleotide. Expression of the sequence encoding the antisense polynucleotide can be by any promoter known in the art to act in mammalian, preferably human cells. Such promoters can be inducible or constitutive. Such promoters include but are not limited to: the SV40 early promoter region (Bernoist and Chambon, Nature 290:304-310 (1981)), the promoter contained in the 3′ long terminal repeat of Rous sarcoma virus (Yamamoto et al., Cell 22:787-797 (1980)), the herpes thymidine kinase promoter (Wagner et al., Proc Natl Acad Sci USA 78:1441-1445 (1981)), the regulatory sequences of the metallothionine gene (Brinster et al., Nature 296:3 S 42 (1982)), etc. Any type of plasmid, cosmid, YAC or viral vector can be used to prepare the recombinant DNA construct that can be introduced directly into the tissue site. Alternatively, viral-vectors can be used which selectively infect the desired tissue, in which case administration may be accomplished by another route (e.g., systemically).

RNAi Constructs—siRNAs and miRNAs

RNA interference (RNAi) is a phenomenon describing double-stranded (ds)RNA-dependent gene specific posttranscriptional silencing. Initial attempts to harness this phenomenon for experimental manipulation of mammalian cells were foiled by a robust and nonspecific antiviral defense mechanism activated in response to long dsRNA molecules (Gil et al., Apoptosis 2000, 5: 107-114). The field was significantly advanced upon the demonstration that synthetic duplexes of 21 nucleotide RNAs could mediate gene specific RNAi in mammalian cells, without invoking generic antiviral defense mechanisms (Elbashir et al., Nature 2001, 411:494-498; Caplen et al., Proc Natl Acad Sci 2001, 98:9742-9747). As a result, small-interfering RNAs (siRNAs) and micro RNAs (miRNAs) have become powerful tools to dissect gene function. The chemical synthesis of small RNAs is one avenue that has produced promising results. Numerous groups have also sought the development of DNA-based vectors capable of generating such siRNA within cells. Several groups have recently attained this goal and published similar strategies that, in general, involve transcription of short hairpin (sh)RNAs that are efficiently processed to form siRNAs within cells (Paddison et al., PNAS 2002, 99:1443-1448; Paddison et al., Genes & Dev 2002, 16:948-958; Sui et al., PNAS 2002, 8:5515-5520; and Brummelkamp et al., Science 2002, 296:550-553). These reports describe methods to generate siRNAs capable of specifically targeting numerous endogenously and exogenously expressed genes.

Accordingly, the present invention provides a polynucleotide comprising an RNAi sequence that acts through an RNAi or miRNA mechanism to attenuate expression of a gene selected from Table I or II. For instance, a polynucleotide of the invention may comprise a miRNA or siRNA sequence that attenuates or inhibits expression of a CCL28 gene. In one embodiment, the miRNA or siRNA sequence is between about 19 nucleotides and about 75 nucleotides in length, or preferably, between about 25 base pairs and about 35 base pairs in length. In certain embodiments, the polynucleotide is a hairpin loop or stem-loop that may be processed by RNAse enzymes (e.g., Drosha and Dicer). An RNAi construct contains a nucleotide sequence that hybridizes under physiologic conditions of the cell to the nucleotide sequence of at least a portion of the mRNA transcript for HTRA1 gene. The double-stranded RNA need only be sufficiently similar to natural RNA that it has the ability to mediate RNAi. The number of tolerated nucleotide mismatches between the target sequence and the RNAi construct sequence is no more than 1 in 5 basepairs, or 1 in 10 basepairs, or 1 in 20 basepairs, or 1 in 50 basepairs. It is primarily important the that RNAi construct is able to specifically target the selected gene from Table I or II. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of polynucleotides comprising RNAi sequences can be carried out by any of the methods for producing polynucleotides described herein. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. Polynucleotides of the invention, including wildtype or antisense polynucleotides, or those that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. Polynucleotides of the invention may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration). The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxy 1 group.

In other embodiments, the subject RNAi constructs are “miRNAs.” microRNAs (miRNAs) are small non-coding RNAs that direct post transcriptional regulation of gene expression through interaction with homologous mRNAs. miRNAs control the expression of genes by binding to complementary sites in target mRNAs from protein coding genes. miRNAs are similar to siRNAs. miRNAs are processed by nucleolytic cleavage from larger double-stranded precursor molecules. These precursor molecules are often hairpin structures of about 70 nucleotides in length, with 25 or more nucleotides that are base-paired in the hairpin. The RNAse III-like enzymes Drosha and Dicer (which may also be used in siRNA processing) cleave the miRNA precursor to produce an miRNA. The processed miRNA is single-stranded and incorporates into a protein complex, termed RISC or miRNP. This RNA-protein complex targets a complementary mRNA. miRNAs inhibit translation or direct cleavage of target mRNAs (Brennecke et al., Genome Biology 4:228 (2003); Kim et al., Mol. Cells 19: 1-15 (2005).

The miRNA and siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA and miRNA molecules. In addition, chromatography (e.g., size exclusion chromatography), glycerol gradient centrifugation, affinity purification with antibody can be used to purify siRNAs and miRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythyinidine is tolerated and does not affect the efficiency of RNAi. The absence of a T hydroxyl significantly enhances the nuclease resistance of the overhang in tissue culture medium and may be beneficial in vivo.

In certain embodiments, a polynucleotide of the invention that comprises an RNAi sequence or an RNAi precursor is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, (Paddison et al., Genes Dev, 2002, 16:948-58; McCaffrey et al, Nature, 2002, 418:38-9; McManus et al., RNA 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that miRNAs and siRNAs can be produced by processing a hairpin RNA in the cell.

In yet other embodiments, a plasmid is used to deliver the double-stranded RNA, e.g., as a transcriptional product. After the coding sequence is transcribed, the complementary RNA transcripts base-pair to form the double-stranded RNA. Several RNAi constructs specifically targeting HTRA1 are commercially available (for example Stealth Select RNAi from Invitrogen).

### Aptamers and Small Molecules

The present invention also provides therapeutic aptamers that specifically bind to a variant polypeptide encoded by a gene selected from Table I or II, thereby modulating activity of said polypeptide. An “aptamer” may be a nucleic acid molecule, such as RNA or DNA that is capable of binding to a specific molecule with high affinity and specificity (Ellington et al., Nature 346, 818-22 (1990); and Tuerk et al., Science 249, 505-10 (1990)). An aptamer will most typically have been obtained by in vitro selection for binding of a target molecule. For example, an aptamer that specifically binds a variant polypeptide encoded by a gene selected from Table I or II can be obtained by in vitro selection for binding to the polypeptide from a pool of polynucleotides. However, in vivo selection of an aptamer is also possible. Aptamers have specific binding regions which are capable of forming complexes with an intended target molecule in an environment wherein other substances in the same environment are not complexed to the nucleic acid. The specificity of the binding is defined in terms of the comparative dissociation constants (Kd) of the aptamer for its ligand (e.g., the selected polypeptide) as compared to the dissociation constant of the aptamer for other materials in the environment or unrelated molecules in general. A ligand (e.g., selected polypeptide) is one which binds to the aptamer with greater affinity than to unrelated material. Typically, the Kd for the aptamer with respect to its ligand will be at least about 10-fold less than the Kd for the aptamer with unrelated material or accompanying material in the environment. Even more preferably, the Kd will be at least about 50-fold less, more preferably at least about 100-fold less, and most preferably at least about 200-fold less. An aptamer will typically be between about 10 and about 300 nucleotides in length. More commonly, an aptamer will be between about 30 and about 100 nucleotides in length.

Methods for selecting aptamers specific for a target of interest are known in the art. For example, organic molecules, nucleotides, amino acids, polypeptides, target features on cell surfaces, ions, metals, salts, saccharides, have all been shown to be suitable for isolating aptamers that can specifically bind to the respective ligand. For instance, organic dyes such as Hoechst 33258 have been successfully used as target ligands for in vitro aptamer selections (Werstuck and Green, Science 282:296-298 (1998)). Other small organic molecules like dopamine, theophylline, sulforhodamine B, and cellobiose have also been used as ligands in the isolation of aptamers. Aptamers have also been isolated for antibiotics such as kanamycin A, lividomycin, tobramycin, neomycin B, viomycin, chloramphenicol and streptomycin. For a review of aptamers that recognize small molecules, see (Famulok, Science 9:324-9 (1999)).

An aptamer of the invention can be comprised entirely of RNA. In other embodiments of the invention, however, the aptamer can instead be comprised entirely of DNA, or partially of DNA, or partially of other nucleotide analogs. To specifically inhibit translation in vivo, RNA aptamers are preferred. Such RNA aptamers are preferably introduced into a cell as DNA that is transcribed into the RNA aptamer. Alternatively, an RNA aptamer itself can be introduced into a cell. Aptamers are typically developed to bind particular ligands by employing known in vivo or in vitro (most typically, in vitro) selection techniques known as SELEX (Ellington et al., Nature 346, 818-22 (1990); and Tuerk et al., Science 249, 505-10 (1990)). Methods of making aptamers are also described in, for example, (U.S. Pat. No. 5,582,981, PCT Publication No. WO 00/20040, U.S. Pat. No. 5,270,163, Lorsch and Szostak, Biochemistry, 33:973 (1994), Mannironi et al., Biochemistry 36:9726 (1997), Blind, Proc Natl Acad Sci USA 96:3606-3610 (1999), Huizenga and Szostak, Biochemistry, 34:656-665 (1995), PCT Publication Nos. WO 99/54506, WO 99/27133, WO 97/42317 and U.S. Pat. No. 5,756,291).

Generally, in their most basic form, in vitro selection techniques for identifying aptamers involve first preparing a large pool of DNA molecules of the desired length that contain at least some region that is randomized or mutagenized. For instance, a common oligonucleotide pool for aptamer selection might contain a region of 20-100 randomized nucleotides flanked on both ends by an about 15-25 nucleotide long region of defined sequence useful for the binding of PCR primers. The oligonucleotide pool is amplified using standard PCR techniques, although any means that will allow faithful, efficient amplification of selected nucleic acid sequences can be employed. The DNA pool is then in vitro transcribed to produce RNA transcripts. The RNA transcripts may then be subjected to affinity chromatography, although any protocol which will allow selection of nucleic acids based on their ability to bind specifically to another molecule (e.g., a protein or any target molecule) may be used. In the case of affinity chromatography, the transcripts are most typically passed through a column or contacted with magnetic beads or the like on which the target ligand has been immobilized. RNA molecules in the pool which bind to the ligand are retained on the column or bead, while nonbinding sequences are washed away. The RNA molecules which bind the ligand are then reverse transcribed and amplified again by PCR (usually after elution). The selected pool sequences are then put through another round of the same type of selection. Typically, the pool sequences are put through a total of about three to ten iterative rounds of the selection procedure. The cDNA is then amplified, cloned, and sequenced using standard procedures to identify the sequence of the RNA molecules which are capable of acting as aptamers for the target ligand. Once an aptamer sequence has been successfully identified, the aptamer may be further optimized by performing additional rounds of selection starting from a pool of oligonucleotides comprising the mutagenized aptamer sequence. For use in the present invention, the aptamer is preferably selected for ligand binding in the presence of salt concentrations and temperatures which mimic normal physiological conditions.

The unique nature of the in vitro selection process allows for the isolation of a suitable aptamer that binds a desired ligand despite a complete dearth of prior knowledge as to what type of structure might bind the desired ligand. The association constant for the aptamer and associated ligand is preferably such that the ligand functions to bind to the aptamer and have the desired effect at the concentration of ligand obtained upon administration of the ligand. For in vivo use, for example, the association constant should be such that binding occurs well below the concentration of ligand that can be achieved in the serum or other tissue. Preferably, the required ligand concentration for in vivo use is also below that which could have undesired effects on the organism.

The present invention also provides small molecules and antibodies that specifically bind to a variant polypeptide encoded by a gene selected from Table I or II, thereby inhibiting the activity of the variant polypeptide. In another embodiment, the small molecules and antibodies that specifically bind to the variant polypeptide prevent the secretion of the polypeptide out of the producing cell (see Poage R, J Neurophysiol, 82:50-59 (1999) for discussion of steric hindrance through antibody binding and cross-linking of vesicles). Examples of small molecules include, without limitation, drugs, metabolites, intermediates, cofactors, transition state analogs, ions, metals, toxins and natural and synthetic polymers (e.g., proteins, peptides, nucleic acids, polysaccharides, glycoproteins, hormones, receptors and cell surfaces such as cell walls and cell membranes). An inhibitor for HTRA1 activity, NVP-LBG976, is available from Novartis, Basel (see also, Grau S, PNAS, (2005) 102: 6021-6026).

### Antibodies

Another aspect of the invention pertains to antibodies. In one embodiment, an antibody that is specifically reactive with a variant polypeptide encoded by a gene selected from Table I or II may be used to detect the presence of the selected polypeptide or to inhibit activity of a the selected polypeptide. For example, by using immunogens derived from the selected peptide, anti-protein/anti-peptide antisera or monoclonal antibodies can be made by standard protocols (see, for example, Antibodies: A Laboratory Manual ed. by Harlow and Lane (Cold Spring Harbor Press: 1988)). A mammal, such as a mouse, a hamster or rabbit can be immunized with an immunogenic form of a selected peptide, an antigenic fragment which is capable of eliciting an antibody response, or a fusion protein. In a particular embodiment, the inoculated mouse does not express endogenous the selected polypeptide, thus facilitating the isolation of antibodies that would otherwise be eliminated as anti-self antibodies. Techniques for conferring immunogenicity on a protein or peptide include conjugation to carriers or other techniques well known in the art. An immunogenic portion of a selected peptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of the selected polypeptide, antisera can be obtained and, if desired, polyclonal antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the selected polypeptide and monoclonal antibodies isolated from a culture comprising such hybridoma cells. The term “antibody” as used herein is intended to include fragments thereof which are also specifically reactive with a selected polypeptide encoded by a gene selected from Table I or II. Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific, single-chain, and chimeric and humanized molecules having affinity for the selected polypeptide conferred by at least one CDR region of the antibody. In preferred embodiments, the antibody further comprises a label attached thereto and able to be detected (e.g., the label can be a radioisotope, fluorescent compound, enzyme or enzyme co-factor).

In certain embodiments, an antibody of the invention is a monoclonal antibody, and in certain embodiments, the invention makes available methods for generating novel antibodies that bind specifically to a variant polypeptide encoded by a gene selected from Table I or II. For example, a method for generating a monoclonal antibody that binds specifically to said polypeptide may comprise administering to a mouse an amount of an immunogenic composition comprising the polypeptide effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the selected variant polypeptide. Once obtained, a hybridoma can be propagated in a cell culture, optionally in culture conditions where the hybridoma-derived cells produce the monoclonal antibody that binds specifically to the variant polypeptide. The monoclonal antibody may be purified from the cell culture. Antibodies reactive to HTRA1 are commercially available (for example from Imgenex) and are also described in, for example, PCT application No. WO 00/08134.

The term “specifically reactive with” as used in reference to an antibody is intended to mean, as is generally understood in the art, that the antibody is sufficiently selective between the antigen of interest (e.g., a polypeptide encoded by a gene selected from Table I or II) and other antigens that are not of interest that the antibody is useful for, at minimum, detecting the presence of the antigen of interest in a particular type of biological sample. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody-antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ M or less.

In addition, the techniques used to screen antibodies in order to identify a desirable antibody may influence the properties of the antibody obtained. For example, if an antibody is to be used for binding an antigen in solution, it may be desirable to test solution binding. A variety of different techniques are available for testing interaction between antibodies and antigens to identify particularly desirable antibodies. Such techniques include ELISAs, surface plasmon resonance binding assays (e.g., the BIAcore binding assay, BIAcore AB, Uppsala, Sweden), sandwich assays (e.g., the paramagnetic bead system of IGEN International, Inc., Gaithersburg, Md.), western blots, immunoprecipitation assays, and immunohistochemistry.

### VII. Therapeutic Methods

The invention also provides a method for treating or preventing AMD, comprising prophylactically or therapeutically treating an individual identified as having a genetic profile in at least one of the genes shown in Tables I or II indicative of increased risk of development or progression of AMD, wherein the genetic profile comprises one or more single nucleotide polymorphisms selected from Table I or Table II.

In some embodiments, the method of treating or preventing AMD in an individual includes prophylactically or therapeutically treating the individual by inhibiting a variant polypeptide encoded by a gene selected from Table I or II in the individual. A variant polypeptide encoded by a gene selected from Table I or II can be inhibited, for example, by administering an antibody or other protein that binds to the variant polypeptide. Alternatively, the variant polypeptide can be inhibited by administering a nucleic acid inhibiting its expression or activity, such as an inhibitory RNA, a nucleic acid encoding an inhibitory RNA, an antisense nucleic acid, or an aptamer, optionally in combination with a human complement factor H polypeptide, an HTRA1 inhibitor, C3 convertase inhibitor, an angiogenic inhibitor, and/or an anti-VEGF inhibitor.

In one embodiment, an individual with a genetic profile indicative of AMD can be treated by administering a composition comprising a human Complement Factor H polypeptide to an individual in need thereof. In one embodiment, the Factor H polypeptide is encoded by a Factor H protective haplotype. A protective Factor H haplotype can encode an isoleucine residue at amino acid position 62 and/or an amino acid other than a histidine at amino acid position 402. For example, a Factor H polypeptide can comprise an isoleucine residue at amino acid position 62, a tyrosine residue at amino acid position 402, and/or an arginine residue at amino acid position 1210. Exemplary Factor H protective haplotypes include the H2 haplotype or the H4 haplotype (see U.S. Patent Publication 2007/0020647, which is incorporated by reference in its entirety herein). Alternatively, the Factor H polypeptide may be encoded by a Factor H neutral haplotype. A neutral haplotype encodes an amino acid other than an isoleucine at amino acid position 62 and an amino acid other than a histidine at amino acid position 402. Exemplary Factor H neutral haplotypes include the H3 haplotype or the H5 haplotype (see U.S. Patent Publication 2007/0020647).

A therapeutic Factor H polypeptide may be a recombinant protein or it may be purified from blood. A Factor H polypeptide may be administered to the eye by intraocular injection or systemically.

Alternatively, or in addition, an individual with a genetic profile indicative of elevated risk of AMD could be treated by inhibiting the expression or activity of HTRA1. As one example, HTRA1 can be inhibited by administering an antibody or other protein (e.g. an antibody variable domain, an addressable fibronectin protein, etc.) that binds HTRA1. Alternatively, HTRA1 can be inhibited by administering a small molecule that interferes with HTRA1 activity (e.g., an inhibitor of the protease activity of HTRA1) or a nucleic acid inhibiting HTRA1 expression or activity, such as an inhibitory RNA (e.g. a short interfering RNA, a short hairpin RNA, or a microRNA), a nucleic acid encoding an inhibitory RNA, an antisense nucleic acid, or an aptamer that binds HTRA1. See, for example, International Publication No. WO 2007/044897. An inhibitor for HTRA1 activity, NVP-LBG976, is available from Novartis, Basel (see also, Grau S, PNAS, (2005) 102: 6021-6026). Antibodies reactive to HTRA1 are commercially available (for example from Imgenex) and are also described in, for example, PCT application No. WO 00/08134.

Alternatively, or in addition, the method of treating or preventing AMD in an individual includes prophylactically or therapeutically treating the individual by inhibiting Factor B and/or C2 in the individual. Factor B can be inhibited, for example, by administering an antibody or other protein (e.g., an antibody variable domain, an addressable fibronectin protein, etc.) that binds Factor B. Alternatively, Factor B can be inhibited by administering a nucleic acid inhibiting Factor B expression or activity, such as an inhibitory RNA, a nucleic acid encoding an inhibitory RNA, an antisense nucleic acid, or an aptamer, or by administering a small molecule that interferes with Factor B activity (e.g., an inhibitor of the protease activity of Factor B). C2 can be inhibited, for example, by administering an antibody or other protein (e.g., an antibody variable domain, an addressable fibronectin protein, etc.) that binds C2. Alternatively, C2 can be inhibited by administering a nucleic acid inhibiting C2 expression or activity, such as an inhibitory RNA, a nucleic acid encoding an inhibitory RNA, an antisense nucleic acid, or an aptamer, or by administering a small molecule that interferes with C2 activity (e.g., an inhibitor of the protease activity of C2).

In another embodiment, an individual with a genetic profile indicative of AMD (i.e., the individual's genetic profile comprises one or more single nucleotide polymorphisms selected from Table I or Table II) can be treated by administering a composition comprising a C3 convertase inhibitor, e.g., compstatin (See e.g. PCT publication WO 2007/076437). optionally in combination with a therapeutic factor H polypeptide. In another embodiment, an individual with a genetic profile indicative of AMD and who is diagnosed with AMD may be treated with an angiogenic inhibitor such as anecortave acetate (RETAANE®, Alcon), an anti-VEGF inhibitor such as pegaptanib (Macugen®, Eyetech Pharmaceuticals and Pfizer, Inc.) and ranibizumab (Lucentis®, Genentech), and/or verteporfin (Visudyne®, QLT, Inc./Novartis).

### VIII. Authorization of Treatment or Payment for Treatment

The invention also provides a healthcare method comprising paying for, authorizing payment for or authorizing the practice of the method of screening for susceptibility to developing or for predicting the course of progression of AMD in a patient, comprising screening for the presence or absence of a genetic profile in at least one gene shown in Table I or II, wherein the genetic profile comprises one or more single nucleotide polymorphisms selected from Table I or II.

According to the methods of the present invention, a third party, e.g., a hospital, clinic, a government entity, reimbursing party, insurance company (e.g., a health insurance company), HMO, third-party payor, or other entity which pays for, or reimburses medical expenses may authorize treatment, authorize payment for treatment, or authorize reimbursement of the costs of treatment. For example, the present invention relates to a healthcare method that includes authorizing the administration of, or authorizing payment or reimbursement for the administration of, a diagnostic assay for determining an individual's susceptibility for developing or for predicting the course of progression of AMD as disclosed herein. For example, the healthcare method can include authorizing the administration of, or authorizing payment or reimbursement for the administration of, a diagnostic assay to determine an individual's susceptibility for development or progression of AMD comprising screening for the presence or absence of a genetic profile in at least one gene shown in Table I or II, wherein the genetic profile comprises one or more SNPs selected from Table I or II.

### IX. Complement-Related Diseases

The polymorphisms provided herein have a statistically significant association with one or more disorders that involve dysfunction of the complement system. In certain embodiments, an individual may have a genetic predisposition based on their genetic profile to developing more than one disorder associated with dysregulation of the complement system. For example, said individual's genetic profile may comprise one or more polymorphism shown in Table I or II, wherein the genetic profile is informative of AMD and another disease characterized by dysregulation of the complement system. Accordingly, the invention contemplates the use of these polymorphisms for assessing an individual's risk for any complement-related disease or condition, including but not limited to AMD. Other complement-related diseases include membranoproliferative glomerulonephritis type II (MPGNII, also known as dense deposit disease), Barraquer-Simons Syndrome, asthma, lupus erythematosus, glomerulonephritis, various forms of arthritis including rheumatoid arthritis, autoimmune heart disease, multiple sclerosis, inflammatory bowel disease, Celiac disease, diabetes mellitus type 1, Sjögren's syndrome, and ischemia-reperfusion injuries. The complement system is also becoming increasingly implicated in diseases of the central nervous system such as Alzheimer's disease, and other neurodegenerative conditions. Applicant suspects that many patients may die of disease caused in part by dysfunction of the complement cascade well before any symptoms of AMD appear. Accordingly, the invention disclosed herein may well be found to be useful in early diagnosis and risk assessment of other disease, enabling opportunistic therapeutic or prophylactic intervention delaying the onset or development of symptoms of such disease.

The examples of the present invention presented below are provided only for illustrative purposes and not to limit the scope of the invention. Numerous embodiments of the invention within the scope of the claims that follow the examples will be apparent to those of ordinary skill in the art from reading the foregoing text and following examples.

### EXAMPLES

Additional sub-analyses were performed to support data derived from analyses described above in Tables I-II. These include:

Sub-analysis 1: One preliminary sub-analysis was performed on a subset of 2,876 SNPs using samples from 590 AMD cases and 375 controls. It was determined that this sample provided adequate power (>80%) for detecting an association between the selected markers and AMD (for a relative risk of 1.7, a sample size of 500 per group was required, and for a relative risk of 1.5, the sample size was calculated to be 700 per group).

The raw data were prepared for analysis in the following manner: 1) SNPs with more than 5% failed calls were deleted (45 total SNPs); 2) SNPs with no allelic variation were deleted (354 alleles); 3) subjects with more than 5% missing genotypes were deleted (11 subjects); and 4) the 2,876 remaining SNPs were assessed for LD, and only one SNP was retained for each pair with r2>0.90 (631 SNPs dropped, leaving 2245 SNPs for analysis). Genotype associations were assessed using a statistical software program (i.e., SAS® PROC CASECONTROL) and the results were sorted both by genotype p-value and by allelic p-value. For 2,245 SNPs, the Bonferroni—corrected alpha level for significance is 0.00002227. Seventeen markers passed this test. HWE was assessed for each of the 17 selected markers, both with all data combined and by group.

AMD-associated SNPs were further analyzed to determine q-values. Of 2245 SNPs analyzed, 74 SNPs were shown to be associated with AMD at a q-value less than 0.50. The first section of SNPs represent loci that passed the Bonferroni condition. The second section of SNPs were those that didn't make the Bonferroni cut-off, but had q-values less than 0.20; the third section of SNPs had q-values greater than 0.20, but less than 0.50. 16 AMD-associated SNPs, located in the CFH, LOC387715, FHR4, FHR5, PRSS11, PLEKHA1 and FHR2 genes passed the Bonferroni level of adjustment. These results confirm the published associations of the CFH and LOC387715, PLEKHA1 and PRSS11 genes with AMD. 14 additional SNPs located within the FHR5, FHR2, CFH, PRSS11, FHR1, SPOCK3, PLEKHA1, C2, FBN2, TLR3 and SPOCK loci were significantly associated with AMD; these SNPs didn't pass the Bonferroni cut-off, but had q-values less than 0.20 (after adjusting for false discovery rate). In addition, another 27 SNPs were significantly associated with AMD (p<0.05) at q-values between 0.20 and 0.50.

These data confirm existing gene associations in the literature. They also provide evidence that other complement-associated genes (e.g., FHR1, FHR2, FHR4, FHR5) may not be in linkage disequilibrium (LD) with CFH and, if replicated in additional cohorts, may be independently associated with AMD. It is also noted that FHR1, FHR2 and FHR4 are in the same LD bin and further genotyping will be required to identify the gene(s) within this group that drive the detected association with AMD.

Sub-analysis 2: Another sub-analysis was performed on a subset comprised of 516 AMD cases and 298 controls using criteria as described above. A total of 3,266 SNPs in 352 genes from these regions were tested. High significance was detected for previously established AMD-associated genes, as well as for several novel AMD genes. SNPs exhibiting p values<0.01 and difference in allele frequencies>10%, and >5%, are depicted in Table I.

Sub-analysis 3: Another sub-analysis was performed comparing 499 AMD cases to 293 controls; data were assessed for Hardy-Weinberg association, analyzed by Chi Square. Using a cutoff of p<0.005, 40 SNPs were significantly associated with AMD; these included SNPs within genes shown previously to be associated with AMD (CFH/ENSG00000000971, CFHR1, CFHR2, CFHR4, CFHR5, F13B, PLEKHA1, LOC387715 and PRSS11/HTRA1), as well as additional strong associations with CCL28 and ADAM12. The same samples were analyzed also by conditioning on the CFH Y402H SNP to determine how much association remained after accounting for this strongly associated SNP using a Cochran-Armitage Chi Square test for association within a bin and a Mantel-Haenszel test for comparing bins. The significance of association for most markers in the CFH region drops or disappears after stratification for Y402H, but this SNP has no effect on the PLEKHA1, LOC387715, PRSS11/HTRA1, CCL28 or ADAM12. Similarly LOC3877156 SNP rs3750847 has no effect on association on chromosome 1 SNPs, although association with chromosome 10-associated SNPs disappears except for ADAM12. Thus, the ADAM12 association is not in LD with the previously established AMD locus on chromosome 10 (PLEKHA1, LOC387715, and PRSS11/HTRA1 genes). The ADAM12 signal appears to be coming from association with the over 84 group.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

TABLE I
Genes and Polymorphisms Associated with AMD

| | | | Allele Frequencies (percentages): Control Population | | | | |
|---|---|---|---|---|---|---|---|
| | | | Homozygotes | | | | Allele 2 |
| Gene | SNP | Allele 1/Allele 2 | Allele 1 | Allele 2 | Heterozygotes | Allele 1 Overall | Overall |
| ADAM12 | rs1676717 | A/G | 17.6 | 29 | 53.4 | 44.3 | 55.7 |
| ADAM12 | rs1621212 | C/T | 29.7 | 17.2 | 53 | 56.3 | 43.8 |
| ADAM12 | rs12779767 | C/T | 41.9 | 10.8 | 47.3 | 65.5 | 34.5 |
| ADAM12 | rs11244834 | C/T | 10.8 | 41.4 | 47.8 | 34.7 | 65.3 |
| ADAM19 | rs12189024 | A/G | 6.4 | 59.1 | 34.5 | 23.6 | 76.4 |
| ADAM19 | rs7725839 | A/C | 2 | 75.3 | 22.6 | 13.3 | 86.7 |
| ADAM19 | rs11740315 | A/G | 8.1 | 58.1 | 33.7 | 25.0 | 75.0 |
| ADAM19 | rs7719224 | C/T | 74.9 | 2 | 23.1 | 86.4 | 13.6 |
| ADAM19 | rs6878446 | A/G | 9.5 | 54.1 | 36.5 | 27.7 | 72.3 |
| APBA2 | rs3829467 | C/T | 0.3 | 84.9 | 14.7 | 7.7 | 92.3 |
| APOB | rs12714097 | C/T | 98.6 | 0 | 1.4 | 99.3 | 0.7 |
| BMP7 | rs6014959 | A/G | 83.4 | 1.4 | 15.3 | 91.0 | 9.0 |
| BMP7 | rs6064517 | C/T | 83.8 | 1 | 15.2 | 91.4 | 8.6 |
| BMP7 | rs162315 | A/G | 5.1 | 64.5 | 30.4 | 20.3 | 79.7 |
| BMP7 | rs162316 | A/G | 5.1 | 64.5 | 30.4 | 20.3 | 79.7 |
| C1Qa | rs172378 | A/G | 34.9 | 18.6 | 46.4 | 58.1 | 41.9 |
| C1RL | rs61917913 | A/G | 0 | 94.9 | 5.1 | 2.5 | 97.5 |
| C4BPA | rs2842706 | A/G | 98.9 | 0 | 1.1 | 99.4 | 0.6 |
| C4BPA | rs1126618 | C/T | 63.5 | 2.4 | 34.1 | 80.6 | 19.4 |
| C5 | rs7033790 | C/T | 68.6 | 3 | 28.4 | 82.8 | 17.2 |
| C5 | rs10739585 | C/G | 68.6 | 3 | 28.4 | 82.8 | 17.2 |
| C5 | rs2230214 | A/G | 2 | 75.3 | 22.6 | 13.3 | 86.7 |
| C5 | rs10985127 | A/G | 61.3 | 4.8 | 33.9 | 78.3 | 21.7 |
| C5 | rs2300932 | A/C | 12.5 | 43.2 | 44.3 | 34.6 | 65.4 |
| C5 | rs12683026 | A/G | 78.4 | 1.7 | 19.9 | 88.3 | 11.7 |
| C5 | rs4837805 | A/G | 43.2 | 11.5 | 45.3 | 65.9 | 34.1 |
| C8A | MRD_4048 | C/G | 99.7 | 0 | 0.3 | 99.8 | 0.2 |
| C8A | MRD_4044 | A/C | 0 | 99.7 | 0.3 | 0.2 | 99.8 |
| CCL28 | rs7380703 | G/T | 4.1 | 62.8 | 33.1 | 20.6 | 79.4 |
| CCL28 | rs11741246 | A/G | 27 | 23.6 | 49.3 | 51.7 | 48.3 |
| CCL28 | rs4443426 | C/T | 24.3 | 27 | 48.6 | 48.6 | 51.4 |
| CLU | MRD_4452 | A/G | 0 | 98 | 2 | 1.0 | 99.0 |
| COL9A1 | rs1135056 | A/G | 28.4 | 17.6 | 54.1 | 55.4 | 44.6 |
| FGFR2 | rs2981582 | C/T | 31.8 | 19.6 | 48.6 | 56.1 | 43.9 |
| FGFR2 | rs2912774 | A/C | 20.6 | 32.1 | 47.3 | 44.3 | 55.7 |
| FGFR2 | rs1319093 | A/T | 2.7 | 66.7 | 30.6 | 18.0 | 82.0 |
| FGFR2 | rs10510088 | A/G | 59.1 | 4.4 | 36.5 | 77.4 | 22.6 |
| FGFR2 | rs12412931 | A/G | 2.7 | 66.9 | 30.4 | 17.9 | 82.1 |
| HABP2 | rs3740532 | C/T | 1.7 | 66.6 | 31.8 | 17.6 | 82.4 |
| HABP2 | rs7080536 | A/G | 0 | 95.2 | 4.8 | 2.4 | 97.6 |
| EMID2 | rs17135580 | C/T | 0.7 | 79 | 20.3 | 10.8 | 89.2 |
| EMID2 | rs12536189 | C/T | 0.7 | 79.1 | 20.3 | 10.8 | 89.2 |
| EMID2 | rs7778986 | A/G | 1.4 | 75.6 | 23 | 12.9 | 87.1 |
| EMID2 | rs11766744 | A/G | 1.7 | 78.6 | 19.7 | 11.5 | 88.5 |
| COL6A3 | rs4663722 | C/G | 81.4 | 2 | 16.6 | 89.7 | 10.3 |
| COL6A3 | rs1874573 | A/G | 48 | 9.8 | 42.2 | 69.1 | 30.9 |
| COL6A3 | rs12992087 | C/T | 68.9 | 0.3 | 30.7 | 84.3 | 15.7 |
| IFNAR2 | rs2826552 | A/T | 11.1 | 46.7 | 42.1 | 32.2 | 67.8 |
| COL4A1 | rs7338606 | C/T | 56.8 | 5.7 | 37.5 | 75.5 | 24.5 |
| COL4A1 | rs11842143 | C/G | 9.5 | 52 | 38.5 | 28.7 | 71.3 |
| COL4A1 | rs595325 | G/T | 4.4 | 72.3 | 23.3 | 16.0 | 84.0 |
| COL4A1 | rs9301441 | C/T | 16.2 | 40.5 | 43.2 | 37.8 | 62.2 |
| COL4A1 | rs754880 | A/G | 14.9 | 34.5 | 50.7 | 40.2 | 59.8 |
| COL4A1 | rs7139492 | C/T | 50.3 | 8.6 | 41.1 | 70.9 | 29.1 |
| COL4A1 | rs72509 | G/T | 3.4 | 67.9 | 28.7 | 17.7 | 82.3 |
| FBLN2 | rs9843344 | A/G | 13.9 | 37.5 | 48.6 | 38.2 | 61.8 |
| FBLN2 | rs1562808 | C/T | 41.8 | 10.2 | 48 | 65.8 | 34.2 |
| FBN2 | rs10057855 | A/G | 1.7 | 85.8 | 12.5 | 7.9 | 92.1 |
| FBN2 | rs10057405 | A/C | 82.4 | 1.7 | 15.9 | 90.4 | 9.6 |
| FBN2 | rs331075 | A/G | 36.5 | 13.2 | 50.3 | 61.7 | 38.3 |
| FBN2 | rs17676236 | C/G | 2 | 81.4 | 16.6 | 10.3 | 89.7 |
| FBN2 | rs6891153 | C/T | 1.4 | 87.8 | 10.8 | 6.8 | 93.2 |
| FBN2 | rs17676260 | C/T | 2 | 81.1 | 16.9 | 10.5 | 89.5 |
| FBN2 | rs154001 | C/T | 10.8 | 51.4 | 37.8 | 29.7 | 70.3 |
| FBN2 | rs3805653 | C/T | 63.2 | 3 | 33.8 | 80.1 | 19.9 |
| FBN2 | rs3828661 | A/C | 63.1 | 3.1 | 33.8 | 80.0 | 20.0 |
| FBN2 | rs11241955 | A/G | 10.8 | 42.6 | 46.6 | 34.1 | 65.9 |
| FBN2 | rs6882394 | C/T | 6.6 | 50.3 | 43.1 | 28.1 | 71.9 |
| FBN2 | rs432792 | C/T | 1.7 | 69.6 | 28.7 | 16.0 | 84.0 |
| FBN2 | rs13181926 | C/T | 62.5 | 3.4 | 34.1 | 79.6 | 20.4 |
| FCN1 | rs10117466 | G/T | 50.2 | 8.8 | 41 | 70.7 | 29.3 |
| FCN1 | rs7857015 | A/G | 46.3 | 9.1 | 44.6 | 68.6 | 31.4 |
| FCN1 | rs2989727 | C/T | 17.9 | 35.8 | 46.3 | 41.0 | 59.0 |
| FCN1 | rs3012788 | C/T | 68.9 | 0.7 | 30.3 | 84.1 | 15.9 |
| HS3ST4 | rs4441276 | A/G | 43.2 | 7.1 | 49.7 | 68.1 | 31.9 |
| HS3ST4 | rs12921387 | C/T | 6.8 | 51.2 | 42 | 27.8 | 72.2 |
| IGLC1 | rs1065464 | C/G | 1.4 | 77.7 | 20.9 | 11.8 | 88.2 |
| IGLC1 | rs4820495 | C/T | 50.7 | 9.8 | 39.5 | 70.4 | 29.6 |
| IL12RB1 | rs273493 | C/T | 92.6 | 0 | 7.4 | 96.3 | 3.7 |
| ITGAX | rs2230429 | C/G | 47.8 | 8.1 | 44.1 | 69.8 | 30.2 |
| ITGAX | rs11574630 | C/T | 49 | 7.8 | 43.2 | 70.6 | 29.4 |
| MASP1 | rs12638131 | G/T | 49.3 | 8.4 | 42.2 | 70.4 | 29.6 |
| MASP2 | rs12142107 | C/T | 94.9 | 0 | 5.1 | 97.4 | 2.6 |
| MYOC | rs2236875 | G/T | 79.7 | 2 | 18.2 | 88.9 | 11.1 |
| MYOC | rs12035960 | C/T | 80.1 | 2 | 17.9 | 89.0 | 11.0 |
| MYOC | rs235868 | A/G | 51.5 | 6.8 | 41.7 | 72.4 | 27.6 |
| PPID | rs8396 | A/G | 58.4 | 6.4 | 35.1 | 76.0 | 24.0 |
| PPID | rs7689418 | G/T | 58.1 | 6.4 | 35.5 | 75.8 | 24.2 |
| PTPRC | rs1932433 | C/T | 35.4 | 17.7 | 46.9 | 58.8 | 41.2 |
| PTPRC | rs17670373 | A/G | 48.6 | 12.2 | 39.2 | 68.2 | 31.8 |
| SLC2A2 | rs7646014 | C/G | 2 | 74 | 24 | 14.0 | 86.0 |
| SLC2A2 | rs1604038 | C/T | 46.6 | 8.8 | 44.6 | 68.9 | 31.1 |
| SLC2A2 | rs5400 | C/T | 74.3 | 2 | 23.6 | 86.1 | 13.9 |
| SLC2A2 | rs11721319 | A/G | 2 | 74.7 | 23.3 | 13.7 | 86.3 |
| SPOCK | rs1229729 | A/G | 31.4 | 24.3 | 44.3 | 53.5 | 46.5 |
| SPOCK | rs1229731 | A/G | 24.3 | 31.1 | 44.6 | 46.6 | 53.4 |
| SPOCK | rs2961633 | A/G | 19.7 | 32.9 | 47.5 | 43.4 | 56.6 |
| SPOCK | rs2961632 | C/T | 33.7 | 18.7 | 47.6 | 57.5 | 42.5 |
| SPOCK | rs12656717 | A/G | 18.9 | 29.4 | 51.7 | 44.8 | 55.2 |
| TGFBR2 | rs4955212 | C/T | 52 | 9.8 | 38.2 | 71.1 | 28.9 |
| TGFBR2 | rs1019855 | C/T | 0.3 | 80.7 | 19 | 9.8 | 90.2 |
| TGFBR2 | rs2082225 | A/G | 80.3 | 0.3 | 19.3 | 90.0 | 10.0 |
| TGFBR2 | rs9823731 | A/G | 16.9 | 35.8 | 47.3 | 40.5 | 59.5 |

| | | | | | | | | Frequencies |
|---|---|---|---|---|---|---|---|---|
| | | Allele Frequencies | | | | | Genotype- | Chi Square |
| | | (percentages): Disease Population | | | | | Likelihood | (both |
| | | Homozygotes | | | Allele 1 | Allele 2 | Ratio (3 | collapsed-2 |
| Gene | SNP | Allele 1 | Allele 2 | Heterozygotes | Overall | Overall | categories) | categories) |
| ADAM12 | rs1676717 | 13.5 | 41.2 | 45.3 | 36.1 | 63.9 | 2.16E−03 | 1.31E−03 |
| ADAM12 | rs1621212 | 40.8 | 13.5 | 45.7 | 63.7 | 36.3 | 6.13E−03 | 3.33E−03 |
| ADAM12 | rs12779767 | 34.7 | 15.4 | 49.9 | 59.6 | 40.4 | 5.33E−02 | 1.83E−02 |
| ADAM12 | rs11244834 | 15.4 | 34.7 | 49.9 | 40.4 | 59.6 | 6.87E−02 | 2.49E−02 |
| ADAM19 | rs12189024 | 10.1 | 48.3 | 41.6 | 30.9 | 69.1 | 8.23E−03 | 1.88E−03 |
| ADAM19 | rs7725839 | 4.4 | 66.9 | 28.8 | 18.8 | 81.3 | 2.06E−02 | 5.18E−03 |
| ADAM19 | rs11740315 | 10.5 | 47.5 | 42.0 | 31.5 | 68.5 | 2.61E−02 | 1.05E−02 |
| ADAM19 | rs7719224 | 67.1 | 4.4 | 28.5 | 81.4 | 18.6 | 3.24E−02 | 9.00E−03 |
| ADAM19 | rs6878446 | 11.5 | 45.3 | 43.2 | 33.1 | 66.9 | 5.85E−02 | 2.51E−02 |
| APBA2 | rs3829467 | 1.8 | 78.9 | 19.3 | 11.5 | 88.5 | 3.25E−02 | 1.67E−02 |
| APOB | rs12714097 | 100.0 | 0.0 | 0.0 | 100.0 | 0.0 | 4.68E−03 | 8.91E−03 |
| BMP7 | rs6014959 | 75.8 | 1.6 | 22.6 | 87.1 | 12.9 | 3.51E−02 | 1.77E−02 |
| BMP7 | rs6064517 | 76.4 | 1.6 | 22.0 | 87.4 | 12.6 | 4.31E−02 | 1.49E−02 |
| BMP7 | rs162315 | 6.9 | 56.0 | 37.0 | 25.4 | 74.6 | 5.71E−02 | 1.84E−02 |
| BMP7 | rs162316 | 6.7 | 56.0 | 37.2 | 25.3 | 74.7 | 5.89E−02 | 2.07E−02 |
| C1Qa | rs172378 | 42.1 | 13.3 | 44.5 | 64.4 | 35.6 | 4.93E−02 | 1.26E−02 |
| C1RL | rs61917913 | 0.0 | 91.1 | 8.9 | 4.5 | 95.5 | 3.97E−02 | 4.97E−02 |
| C4BPA | rs2842706 | 100.0 | 0.0 | 0.0 | 100.0 | 0.0 | 1.37E−02 | 2.20E−02 |
| C4BPA | rs1126618 | 71.4 | 2.2 | 26.4 | 84.6 | 15.4 | 6.43E−02 | 3.68E−02 |
| C5 | rs7033790 | 62.2 | 7.3 | 30.5 | 77.4 | 22.6 | 1.80E−02 | 1.07E−02 |
| C5 | rs10739585 | 62.2 | 7.3 | 30.5 | 77.4 | 22.6 | 1.80E−02 | 1.07E−02 |
| C5 | rs2230214 | 1.4 | 82.8 | 15.8 | 9.3 | 90.7 | 4.22E−02 | 1.20E−02 |
| C5 | rs10985127 | 69.9 | 3.2 | 26.9 | 83.3 | 16.7 | 4.42E−02 | 1.20E−02 |
| C5 | rs2300932 | 17.2 | 35.8 | 46.9 | 40.7 | 59.3 | 5.84E−02 | 1.60E−02 |
| C5 | rs12683026 | 84.6 | 0.8 | 14.7 | 91.9 | 8.1 | 7.39E−02 | 1.94E−02 |
| C5 | rs4837805 | 37.2 | 15.8 | 46.9 | 60.7 | 39.3 | 1.13E−01 | 3.84E−02 |
| C8A | MRD_4048 | 97.4 | 0.0 | 2.6 | 98.7 | 1.3 | 8.80E−03 | 2.04E−02 |
| C8A | MRD_4044 | 0.0 | 97.4 | 2.6 | 1.3 | 98.7 | 9.03E−03 | 2.08E−02 |
| CCL28 | rs7380703 | 10.1 | 50.2 | 39.7 | 30.0 | 70.0 | 1.87E−04 | 4.27E−05 |
| CCL28 | rs11741246 | 22.4 | 31.5 | 46.0 | 45.4 | 54.6 | 4.46E−02 | 1.56E−02 |
| CCL28 | rs4443426 | 31.5 | 22.0 | 46.5 | 54.8 | 45.2 | 6.27E−02 | 1.82E−02 |
| CLU | MRD_4452 | 0.0 | 94.7 | 5.3 | 2.7 | 97.3 | 1.62E−02 | 2.40E−02 |
| COL9A1 | rs1135056 | 38.3 | 16.9 | 44.8 | 60.7 | 39.3 | 1.27E−02 | 3.73E−02 |
| FGFR2 | rs2981582 | 41.6 | 13.7 | 44.8 | 64.0 | 36.0 | 8.59E−03 | 1.80E−03 |
| FGFR2 | rs2912774 | 14.5 | 40.6 | 45.0 | 36.9 | 63.1 | 1.82E−02 | 3.81E−03 |
| FGFR2 | rs1319093 | 2.4 | 74.3 | 23.4 | 14.1 | 85.9 | 7.17E−02 | 3.46E−02 |
| FGFR2 | rs10510088 | 67.1 | 3.8 | 29.1 | 81.7 | 18.3 | 7.41E−02 | 3.67E−02 |
| FGFR2 | rs12412931 | 2.4 | 74.3 | 23.4 | 14.1 | 85.9 | 8.22E−02 | 4.00E−02 |
| HABP2 | rs3740532 | 4.2 | 58.7 | 37.1 | 22.7 | 77.3 | 2.65E−02 | 1.43E−02 |
| HABP2 | rs7080536 | 0.2 | 90.9 | 8.9 | 4.7 | 95.3 | 4.99E−02 | 2.14E−02 |
| EMID2 | rs17135580 | 2.4 | 70.9 | 26.7 | 15.7 | 84.3 | 1.51E−02 | 6.38E−03 |
| EMID2 | rs12536189 | 2.4 | 71.0 | 26.6 | 15.7 | 84.3 | 1.55E−02 | 6.58E−03 |
| EMID2 | rs7778986 | 2.7 | 68.2 | 29.2 | 17.2 | 82.8 | 6.35E−02 | 2.18E−02 |
| EMID2 | rs11766744 | 2.2 | 71.8 | 26.0 | 15.2 | 84.8 | 9.59E−02 | 3.97E−02 |
| COL6A3 | rs4663722 | 86.5 | 0.6 | 12.9 | 92.9 | 7.1 | 6.42E−02 | 2.28E−02 |
| COL6A3 | rs1874573 | 36.4 | 12.5 | 51.1 | 62.0 | 38.0 | 5.84E−03 | 4.09E−03 |
| COL6A3 | rs12992087 | 65.9 | 3.4 | 30.7 | 81.3 | 18.7 | 6.10E−03 | 1.28E−01 |
| IFNAR2 | rs2826552 | 12.4 | 35.4 | 52.2 | 38.5 | 61.5 | 9.90E−03 | 1.55E−02 |
| COL4A1 | rs7338606 | 68.1 | 3.6 | 28.3 | 82.3 | 17.7 | 4.86E−03 | 1.13E−03 |
| COL4A1 | rs11842143 | 13.9 | 41.0 | 45.1 | 36.4 | 63.6 | 6.83E−03 | 1.59E−03 |
| COL4A1 | rs595325 | 5.6 | 63.3 | 31.2 | 21.1 | 78.9 | 3.14E−02 | 1.28E−02 |
| COL4A1 | rs9301441 | 20.6 | 31.7 | 47.7 | 44.5 | 55.5 | 3.24E−02 | 9.59E−03 |
| COL4A1 | rs754880 | 21.6 | 29.5 | 48.9 | 46.0 | 54.0 | 4.65E−02 | 2.31E−02 |
| COL4A1 | rs7139492 | 58.8 | 5.9 | 35.2 | 76.4 | 23.6 | 5.29E−02 | 1.45E−02 |
| COL4A1 | rs72509 | 2.2 | 74.5 | 23.4 | 13.9 | 86.1 | 1.23E−01 | 3.75E−02 |
| FBLN2 | rs9843344 | 10.1 | 46.5 | 43.4 | 31.8 | 68.2 | 3.06E−02 | 9.19E−03 |
| FBLN2 | rs1562808 | 50.0 | 7.1 | 42.9 | 71.4 | 28.6 | 5.51E−02 | 1.90E−02 |
| FBN2 | rs10057855 | 1.4 | 76.0 | 22.6 | 12.7 | 87.3 | 1.49E−03 | 3.37E−03 |
| FBN2 | rs10057405 | 72.5 | 1.8 | 25.7 | 85.3 | 14.7 | 4.00E−03 | 3.66E−03 |
| FBN2 | rs331075 | 27.7 | 20.8 | 51.5 | 53.5 | 46.5 | 4.32E−03 | 1.42E−03 |
| FBN2 | rs17676236 | 1.6 | 72.7 | 25.7 | 14.5 | 85.5 | 8.92E−03 | 1.68E−02 |
| FBN2 | rs6891153 | 0.8 | 80.6 | 18.7 | 10.1 | 89.9 | 8.93E−03 | 2.24E−02 |
| FBN2 | rs17676260 | 1.6 | 72.5 | 25.9 | 14.6 | 85.4 | 1.07E−02 | 1.92E−02 |
| FBN2 | rs154001 | 13.7 | 40.6 | 45.7 | 36.5 | 63.5 | 1.25E−02 | 5.52E−03 |
| FBN2 | rs3805653 | 54.7 | 4.6 | 40.8 | 75.0 | 25.0 | 5.30E−02 | 2.14E−02 |
| FBN2 | rs3828661 | 54.9 | 4.8 | 40.4 | 75.0 | 25.0 | 5.88E−02 | 2.28E−02 |
| FBN2 | rs11241955 | 7.7 | 49.9 | 42.4 | 28.9 | 71.1 | 8.74E−02 | 2.93E−02 |
| FBN2 | rs6882394 | 9.9 | 44.1 | 46.1 | 32.9 | 67.1 | 1.20E−01 | 4.91E−02 |
| FBN2 | rs432792 | 1.2 | 76.2 | 22.6 | 12.5 | 87.5 | 1.20E−01 | 4.54E−02 |
| FBN2 | rs13181926 | 56.4 | 5.7 | 37.8 | 75.3 | 24.7 | 1.27E−01 | 5.34E−02 |
| FCN1 | rs10117466 | 39.2 | 12.2 | 48.6 | 63.5 | 36.5 | 9.29E−03 | 3.66E−03 |
| FCN1 | rs7857015 | 36.8 | 13.3 | 49.9 | 61.8 | 38.2 | 1.83E−02 | 6.12E−03 |
| FCN1 | rs2989727 | 12.9 | 43.0 | 44.2 | 35.0 | 65.0 | 5.69E−02 | 1.48E−02 |
| FCN1 | rs3012788 | 60.7 | 1.3 | 38.0 | 79.7 | 20.3 | 7.65E−02 | 3.91E−02 |
| HS3ST4 | rs4441276 | 49.2 | 12.1 | 38.7 | 68.6 | 31.4 | 3.35E−03 | 8.43E−01 |
| HS3ST4 | rs12921387 | 11.5 | 45.7 | 42.7 | 32.9 | 67.1 | 5.76E−02 | 3.33E−02 |
| IGLC1 | rs1065464 | 0.0 | 72.9 | 27.1 | 13.5 | 86.5 | 3.33E−03 | 3.22E−01 |
| IGLC1 | rs4820495 | 42.4 | 11.3 | 46.3 | 65.5 | 34.5 | 7.49E−02 | 4.37E−02 |
| IL12RB1 | rs273493 | 86.8 | 0.0 | 13.2 | 93.4 | 6.6 | 1.14E−02 | 1.69E−02 |
| ITGAX | rs2230429 | 42.9 | 14.9 | 42.3 | 64.0 | 36.0 | 1.48E−02 | 1.72E−02 |
| ITGAX | rs11574630 | 42.8 | 13.9 | 43.4 | 64.5 | 35.5 | 1.91E−02 | 1.16E−02 |
| MASP1 | rs12638131 | 57.9 | 7.1 | 34.9 | 75.4 | 24.6 | 6.14E−02 | 2.99E−02 |
| MASP2 | rs12142107 | 97.8 | 0.0 | 2.2 | 98.9 | 1.1 | 2.81E−02 | 2.60E−02 |
| MYOC | rs2236875 | 85.9 | 0.2 | 13.9 | 92.9 | 7.1 | 5.92E−03 | 5.64E−03 |
| MYOC | rs12035960 | 85.9 | 0.2 | 13.9 | 92.9 | 7.1 | 7.27E−03 | 7.80E−03 |
| MYOC | rs235868 | 46.2 | 11.0 | 42.8 | 67.6 | 32.4 | 9.30E−02 | 4.73E−02 |
| PPID | rs8396 | 46.3 | 9.3 | 44.4 | 68.5 | 31.5 | 3.68E−03 | 1.37E−03 |
| PPID | rs7689418 | 46.6 | 9.1 | 44.2 | 68.8 | 31.3 | 6.52E−03 | 2.44E−03 |
| PTPRC | rs1932433 | 42.0 | 9.6 | 48.4 | 66.2 | 33.8 | 3.08E−03 | 3.11E−03 |
| PTPRC | rs17670373 | 37.2 | 12.3 | 50.5 | 62.5 | 37.5 | 4.02E−03 | 1.98E−02 |
| SLC2A2 | rs7646014 | 0.4 | 82.4 | 17.2 | 9.0 | 91.0 | 4.79E−03 | 1.87E−03 |
| SLC2A2 | rs1604038 | 56.7 | 6.2 | 37.1 | 75.3 | 24.7 | 1.81E−02 | 5.56E−03 |
| SLC2A2 | rs5400 | 81.8 | 0.6 | 17.6 | 90.6 | 9.4 | 1.91E−02 | 6.15E−03 |
| SLC2A2 | rs11721319 | 0.6 | 81.7 | 17.7 | 9.4 | 90.6 | 2.48E−02 | 8.59E−03 |
| SPOCK | rs1229729 | 33.5 | 14.9 | 51.7 | 59.3 | 40.7 | 3.70E−03 | 2.45E−02 |
| SPOCK | rs1229731 | 14.9 | 33.5 | 51.7 | 40.7 | 59.3 | 3.91E−03 | 2.07E−02 |
| SPOCK | rs2961633 | 11.6 | 37.4 | 51.0 | 37.1 | 62.9 | 8.54E−03 | 1.32E−02 |
| SPOCK | rs2961632 | 37.8 | 11.5 | 50.7 | 63.2 | 36.8 | 1.95E−02 | 2.46E−02 |
| SPOCK | rs12656717 | 25.0 | 22.0 | 53.1 | 51.5 | 48.5 | 2.74E−02 | 9.39E−03 |
| TGFBR2 | rs4955212 | 60.4 | 5.7 | 33.9 | 77.3 | 22.7 | 2.51E−02 | 5.56E−03 |
| TGFBR2 | rs1019855 | 1.8 | 74.7 | 23.6 | 13.6 | 86.4 | 3.93E−02 | 2.76E−02 |
| TGFBR2 | rs2082225 | 74.7 | 1.8 | 23.6 | 86.4 | 13.6 | 4.72E−02 | 3.59E−02 |
| TGFBR2 | rs9823731 | 13.1 | 42.2 | 44.8 | 35.4 | 64.6 | 1.33E−01 | 4.18E−02 |

TABLE 2
Additional Genes and Polymorphisms associated with AMD

| | | | Allele Frequencies (percentages): Control Population | | | | |
|---|---|---|---|---|---|---|---|
| | | | Homozygotes | | | | Allele 2 |
| Gene | SNP | Allele 1/Allele 2 | Allele 1 | Allele 2 | Heterozygotes | Allele 1 Overall | Overall |
| C3 | rs2547438 | G/T | 6.7 | 52.4 | 40.9 | 27.1 | 72.9 |
| C3 | rs2230199 | C/G | 6.1 | 63.4 | 30.5 | 21.4 | 78.6 |
| C3 | rs1047286 | A/G | 4.9 | 64.1 | 31 | 20.4 | 79.6 |
| C3 | rs3745567 | A/G | 1.4 | 76.1 | 22.5 | 12.6 | 87.4 |
| C3 | rs11569507 | A/G | 76.7 | 1.4 | 22 | 87.7 | 12.3 |
| C3 | rs11085197 | C/G | 5.7 | 63.2 | 31.1 | 21.3 | 78.7 |
| C7 | rs2271708 | A/G | 99.7 | 0 | 0.3 | 99.8 | 0.2 |
| C7 | rs1055021 | A/C | 0 | 82.1 | 17.9 | 9.0 | 91.0 |
| C9 | rs476569 | C/T | 23.6 | 25 | 51.4 | 49.3 | 50.7 |
| C1NH | rs2511988 | A/G | 10.1 | 43.2 | 46.6 | 33.4 | 66.6 |
| C1NH | rs4926 | A/G | 4.7 | 56.8 | 38.5 | 24.0 | 76.0 |
| ITGA4 | rs3770115 | C/T | 40.2 | 12.5 | 47.3 | 63.9 | 36.1 |
| ITGA4 | rs4667319 | A/G | 38.9 | 14.2 | 47 | 62.3 | 37.7 |

| | | | | | | | | Frequencies |
|---|---|---|---|---|---|---|---|---|
| | | Allele Frequencies | | | | | Genotype- | Chi Square |
| | | (percentages): Disease Population | | | | | Likelihood | (both |
| | | Homozygotes | | | Allele 1 | Allele 2 | Ratio (3 | collapsed-2 |
| Gene | SNP | Allele 1 | Allele 2 | Heterozygotes | Overall | Overall | categories) | categories) |
| C3 | rs2547438 | 3.2 | 62.3 | 34.5 | 20.4 | 79.6 | 1.01E−02 | 3.27E−03 |
| C3 | rs2230199 | 8.5 | 55.8 | 35.7 | 26.4 | 73.6 | 8.92E−02 | 2.40E−02 |
| C3 | rs1047286 | 8.2 | 57.5 | 34.3 | 25.4 | 74.6 | 9.08E−02 | 2.75E−02 |
| C3 | rs3745567 | 0.0 | 81.2 | 18.8 | 9.4 | 90.6 | 7.19E−03 | 4.37E−02 |
| C3 | rs11569507 | 81.4 | 0.0 | 18.6 | 90.7 | 9.3 | 8.56E−03 | 5.59E−02 |
| C3 | rs11085197 | 8.1 | 56.6 | 35.2 | 25.7 | 74.3 | 1.48E−01 | 4.40E−02 |
| C7 | rs2271708 | 97.4 | 0.0 | 2.6 | 98.7 | 1.3 | 8.66E−03 | 2.01E−02 |
| C7 | rs1055021 | 2.0 | 81.6 | 16.4 | 10.2 | 89.8 | 8.79E−03 | 4.17E−01 |
| C9 | rs476569 | 31.9 | 19.2 | 48.9 | 56.3 | 43.7 | 2.23E−02 | 6.59E−03 |
| C1NH | rs2511988 | 6.3 | 51.2 | 42.5 | 27.6 | 72.4 | 3.79E−02 | 1.32E−02 |
| C1NH | rs4926 | 8.1 | 45.9 | 45.9 | 31.1 | 68.9 | 6.66E−03 | 2.36E−03 |
| ITGA4 | rs3770115 | 51.9 | 9.7 | 38.4 | 71.1 | 28.9 | 5.83E−03 | 2.63E−03 |
| ITGA4 | rs4667319 | 48.1 | 11.7 | 40.2 | 68.2 | 31.8 | 3.79E−02 | 1.63E−02 |

TABLE III A
AMD Control Population Cases

| | | | | | Allele Frequencies: | | | | | | | |
|---|---|---|---|---|---|---|---|---|---|---|---|---|
| | | Allele | Control | | Control Population | | | Allele Frequencies (percentages): Control Population | | | | |
| | | 1/ | Undeter. | | Homozygotes | | | Homozygotes | | | Allele 1 | Allele 2 |
| Gene | SNP | Allele 2 | Frequency | Control N | Allele 1 | Allele 2 | Heterozygotes | Allele 1 | Allele 2 | Heterozygotes | Overall | Overall |
| ADAM12 | rs1676717 | A/G | 6 | 290 | 51 | 84 | 155 | 17.6 | 29 | 53.4 | 44.3 | 55.7 |
| ADAM12 | rs1621212 | C/T | 0 | 296 | 88 | 51 | 157 | 29.7 | 17.2 | 53 | 56.3 | 43.8 |
| ADAM12 | rs12779767 | C/T | 0 | 296 | 124 | 32 | 140 | 41.9 | 10.8 | 47.3 | 65.5 | 34.5 |
| ADAM12 | rs11244834 | C/T | 1 | 295 | 32 | 122 | 141 | 10.8 | 41.4 | 47.8 | 34.7 | 65.3 |
| ADAM19 | rs12189024 | A/G | 0 | 296 | 19 | 175 | 102 | 6.4 | 59.1 | 34.5 | 23.6 | 76.4 |
| ADAM19 | rs7725839 | A/C | 0 | 296 | 6 | 223 | 67 | 2 | 75.3 | 22.6 | 13.3 | 86.7 |
| ADAM19 | rs11740315 | A/G | 50 | 246 | 20 | 143 | 83 | 8.1 | 58.1 | 33.7 | 25.0 | 75.0 |
| ADAM19 | rs7719224 | C/T | 1 | 295 | 221 | 6 | 68 | 74.9 | 2 | 23.1 | 86.4 | 13.6 |
| ADAM19 | rs6878446 | A/G | 0 | 296 | 28 | 160 | 108 | 9.5 | 54.1 | 36.5 | 27.7 | 72.3 |
| APBA2 | rs3829467 | C/T | 4 | 292 | 1 | 248 | 43 | 0.3 | 84.9 | 14.7 | 7.7 | 92.3 |
| APOB | rs12714097 | C/T | 0 | 296 | 292 | 0 | 4 | 98.6 | 0 | 1.4 | 99.3 | 0.7 |
| BMP7 | rs6014959 | A/G | 1 | 295 | 246 | 4 | 45 | 83.4 | 1.4 | 15.3 | 91.0 | 9.0 |
| BMP7 | rs6064517 | C/T | 0 | 296 | 248 | 3 | 45 | 83.8 | 1 | 15.2 | 91.4 | 8.6 |
| BMP7 | rs162315 | A/G | 0 | 296 | 15 | 191 | 90 | 5.1 | 64.5 | 30.4 | 20.3 | 79.7 |
| BMP7 | rs162316 | A/G | 0 | 296 | 15 | 191 | 90 | 5.1 | 64.5 | 30.4 | 20.3 | 79.7 |
| C1Qa | rs172378 | A/G | 1 | 295 | 103 | 55 | 137 | 34.9 | 18.6 | 46.4 | 58.1 | 41.9 |
| C1RL | rs61917913 | A/G | 0 | 296 | 0 | 281 | 15 | 0 | 94.9 | 5.1 | 2.5 | 97.5 |
| C4BPA | rs2842706 | A/G | 25 | 271 | 268 | 0 | 3 | 98.9 | 0 | 1.1 | 99.4 | 0.6 |
| C4BPA | rs1126618 | C/T | 0 | 296 | 188 | 7 | 101 | 63.5 | 2.4 | 34.1 | 80.6 | 19.4 |
| C5 | rs7033790 | C/T | 0 | 296 | 203 | 9 | 84 | 68.6 | 3 | 28.4 | 82.8 | 17.2 |
| C5 | rs10739585 | C/G | 0 | 296 | 203 | 9 | 84 | 68.6 | 3 | 28.4 | 82.8 | 17.2 |
| C5 | rs2230214 | A/G | 0 | 296 | 6 | 223 | 67 | 2 | 75.3 | 22.6 | 13.3 | 86.7 |
| C5 | rs10985127 | A/G | 4 | 292 | 179 | 14 | 99 | 61.3 | 4.8 | 33.9 | 78.3 | 21.7 |
| C5 | rs2300932 | A/C | 0 | 296 | 37 | 128 | 131 | 12.5 | 43.2 | 44.3 | 34.6 | 65.4 |
| C5 | rs12683026 | A/G | 0 | 296 | 232 | 5 | 59 | 78.4 | 1.7 | 19.9 | 88.3 | 11.7 |
| C5 | rs4837805 | A/G | 0 | 296 | 128 | 34 | 134 | 43.2 | 11.5 | 45.3 | 65.9 | 34.1 |
| C8A | MRD_4048 | C/G | 1 | 295 | 294 | 0 | 1 | 99.7 | 0 | 0.3 | 99.8 | 0.2 |
| C8A | MRD_4044 | A/C | 2 | 294 | 0 | 293 | 1 | 0 | 99.7 | 0.3 | 0.2 | 99.8 |
| CCL28 | rs7380703 | G/T | 0 | 296 | 12 | 186 | 98 | 4.1 | 62.8 | 33.1 | 20.6 | 79.4 |
| CCL28 | rs11741246 | A/G | 0 | 296 | 80 | 70 | 146 | 27 | 23.6 | 49.3 | 51.7 | 48.3 |
| CCL28 | rs4443426 | C/T | 0 | 296 | 72 | 80 | 144 | 24.3 | 27 | 48.6 | 48.6 | 51.4 |
| CLU | MRD_4452 | A/G | 0 | 296 | 0 | 290 | 6 | 0 | 98 | 2 | 1.0 | 99.0 |
| COL9A1 | rs1135056 | A/G | 0 | 296 | 84 | 52 | 160 | 28.4 | 17.6 | 54.1 | 55.4 | 44.6 |
| FGFR2 | rs2981582 | C/T | 0 | 296 | 94 | 58 | 144 | 31.8 | 19.6 | 48.6 | 56.1 | 43.9 |
| FGFR2 | rs2912774 | A/C | 0 | 296 | 61 | 95 | 140 | 20.6 | 32.1 | 47.3 | 44.3 | 55.7 |
| FGFR2 | rs1319093 | A/T | 2 | 294 | 8 | 196 | 90 | 2.7 | 66.7 | 30.6 | 18.0 | 82.0 |
| FGFR2 | rs10510088 | A/G | 0 | 296 | 175 | 13 | 108 | 59.1 | 4.4 | 36.5 | 77.4 | 22.6 |
| FGFR2 | rs12412931 | A/G | 0 | 296 | 8 | 198 | 90 | 2.7 | 66.9 | 30.4 | 17.9 | 82.1 |
| HABP2 | rs3740532 | C/T | 0 | 296 | 5 | 197 | 94 | 1.7 | 66.6 | 31.8 | 17.6 | 82.4 |
| HABP2 | rs7080536 | A/G | 2 | 294 | 0 | 280 | 14 | 0 | 95.2 | 4.8 | 2.4 | 97.6 |
| EMID2 | rs17135580 | C/T | 1 | 295 | 2 | 233 | 60 | 0.7 | 79 | 20.3 | 10.8 | 89.2 |
| EMID2 | rs12536189 | C/T | 0 | 296 | 2 | 234 | 60 | 0.7 | 79.1 | 20.3 | 10.8 | 89.2 |
| EMID2 | rs7778986 | A/G | 5 | 291 | 4 | 220 | 67 | 1.4 | 75.6 | 23 | 12.9 | 87.1 |
| EMID2 | rs11766744 | A/G | 1 | 295 | 5 | 232 | 58 | 1.7 | 78.6 | 19.7 | 11.5 | 88.5 |
| COL6A3 | rs4663722 | C/G | 0 | 296 | 241 | 6 | 49 | 81.4 | 2 | 16.6 | 89.7 | 10.3 |
| COL6A3 | rs1874573 | A/G | 0 | 296 | 142 | 29 | 125 | 48 | 9.8 | 42.2 | 69.1 | 30.9 |
| COL6A3 | rs12992087 | C/T | 0 | 296 | 204 | 1 | 91 | 68.9 | 0.3 | 30.7 | 84.3 | 15.7 |
| IFNAR2 | rs2826552 | A/T | 35 | 261 | 29 | 122 | 110 | 11.1 | 46.7 | 42.1 | 32.2 | 67.8 |
| COL4A1 | rs7338606 | C/T | 0 | 296 | 168 | 17 | 111 | 56.8 | 5.7 | 37.5 | 75.5 | 24.5 |
| COL4A1 | rs11842143 | C/G | 0 | 296 | 28 | 154 | 114 | 9.5 | 52 | 38.5 | 28.7 | 71.3 |
| COL4A1 | rs595325 | G/T | 0 | 296 | 13 | 214 | 69 | 4.4 | 72.3 | 23.3 | 16.0 | 84.0 |
| COL4A1 | rs9301441 | C/T | 0 | 296 | 48 | 120 | 128 | 16.2 | 40.5 | 43.2 | 37.8 | 62.2 |
| COL4A1 | rs754880 | A/G | 0 | 296 | 44 | 102 | 150 | 14.9 | 34.5 | 50.7 | 40.2 | 59.8 |
| COL4A1 | rs7139492 | C/T | 4 | 292 | 147 | 25 | 120 | 50.3 | 8.6 | 41.1 | 70.9 | 29.1 |
| COL4A1 | rs72509 | G/T | 0 | 296 | 10 | 201 | 85 | 3.4 | 67.9 | 28.7 | 17.7 | 82.3 |
| FBLN2 | rs9843344 | A/G | 0 | 296 | 41 | 111 | 144 | 13.9 | 37.5 | 48.6 | 38.2 | 61.8 |
| FBLN2 | rs1562808 | C/T | 2 | 294 | 123 | 30 | 141 | 41.8 | 10.2 | 48 | 65.8 | 34.2 |
| FBN2 | rs10057855 | A/G | 0 | 296 | 5 | 254 | 37 | 1.7 | 85.8 | 12.5 | 7.9 | 92.1 |
| FBN2 | rs10057405 | A/C | 0 | 296 | 244 | 5 | 47 | 82.4 | 1.7 | 15.9 | 90.4 | 9.6 |
| FBN2 | rs331075 | A/G | 0 | 296 | 108 | 39 | 149 | 36.5 | 13.2 | 50.3 | 61.7 | 38.3 |
| FBN2 | rs17676236 | C/G | 0 | 296 | 6 | 241 | 49 | 2 | 81.4 | 16.6 | 10.3 | 89.7 |
| FBN2 | rs6891153 | C/T | 0 | 296 | 4 | 260 | 32 | 1.4 | 87.8 | 10.8 | 6.8 | 93.2 |
| FBN2 | rs17676260 | C/T | 0 | 296 | 6 | 240 | 50 | 2 | 81.1 | 16.9 | 10.5 | 89.5 |
| FBN2 | rs154001 | C/T | 0 | 296 | 32 | 152 | 112 | 10.8 | 51.4 | 37.8 | 29.7 | 70.3 |
| FBN2 | rs3805653 | C/T | 0 | 296 | 187 | 9 | 100 | 63.2 | 3 | 33.8 | 80.1 | 19.9 |
| FBN2 | rs3828661 | A/C | 3 | 293 | 185 | 9 | 99 | 63.1 | 3.1 | 33.8 | 80.0 | 20.0 |
| FBN2 | rs3828661 | A/C | 3 | 293 | 185 | 9 | 99 | 63.1 | 3.1 | 33.8 | 80.0 | 20.0 |
| FBN2 | rs11241955 | A/G | 0 | 296 | 32 | 126 | 138 | 10.8 | 42.6 | 46.6 | 34.1 | 65.9 |
| FBN2 | rs6882394 | C/T | 8 | 288 | 19 | 145 | 124 | 6.6 | 50.3 | 43.1 | 28.1 | 71.9 |
| FBN2 | rs432792 | C/T | 0 | 296 | 5 | 206 | 85 | 1.7 | 69.6 | 28.7 | 16.0 | 84.0 |
| FBN2 | rs13181926 | C/T | 0 | 296 | 185 | 10 | 101 | 62.5 | 3.4 | 34.1 | 79.6 | 20.4 |
| FCN1 | rs10117466 | G/T | 1 | 295 | 148 | 26 | 121 | 50.2 | 8.8 | 41 | 70.7 | 29.3 |
| FCN1 | rs7857015 | A/G | 0 | 296 | 137 | 27 | 132 | 46.3 | 9.1 | 44.6 | 68.6 | 31.4 |
| FCN1 | rs2989727 | C/T | 0 | 296 | 53 | 106 | 137 | 17.9 | 35.8 | 46.3 | 41.0 | 59.0 |
| FCN1 | rs3012788 | C/T | 29 | 267 | 184 | 2 | 81 | 68.9 | 0.7 | 30.3 | 84.1 | 15.9 |
| HS3ST4 | rs4441276 | A/G | 0 | 296 | 128 | 21 | 147 | 43.2 | 7.1 | 49.7 | 68.1 | 31.9 |
| HS3ST4 | rs12921387 | C/T | 1 | 295 | 20 | 151 | 124 | 6.8 | 51.2 | 42 | 27.8 | 72.2 |
| IGLC1 | rs1065464 | C/G | 0 | 296 | 4 | 230 | 62 | 1.4 | 77.7 | 20.9 | 11.8 | 88.2 |
| IGLC1 | rs4820495 | C/T | 0 | 296 | 150 | 29 | 117 | 50.7 | 9.8 | 39.5 | 70.4 | 29.6 |
| IL12RB1 | rs273493 | C/T | 13 | 283 | 262 | 0 | 21 | 92.6 | 0 | 7.4 | 96.3 | 3.7 |
| ITGAX | rs2230429 | C/G | 1 | 295 | 141 | 24 | 130 | 47.8 | 8.1 | 44.1 | 69.8 | 30.2 |
| ITGAX | rs11574630 | C/T | 0 | 296 | 145 | 23 | 128 | 49 | 7.8 | 43.2 | 70.6 | 29.4 |
| MASP1 | rs12638131 | G/T | 0 | 296 | 146 | 25 | 125 | 49.3 | 8.4 | 42.2 | 70.4 | 29.6 |
| MASP2 | rs12142107 | C/T | 3 | 293 | 278 | 0 | 15 | 94.9 | 0 | 5.1 | 97.4 | 2.6 |
| MYOC | rs2236875 | G/T | 0 | 296 | 236 | 6 | 54 | 79.7 | 2 | 18.2 | 88.9 | 11.1 |
| MYOC | rs12035960 | C/T | 0 | 296 | 237 | 6 | 53 | 80.1 | 2 | 17.9 | 89.0 | 11.0 |
| MYOC | rs235868 | A/G | 1 | 295 | 152 | 20 | 123 | 51.5 | 6.8 | 41.7 | 72.4 | 27.6 |
| PPID | rs8396 | A/G | 0 | 296 | 173 | 19 | 104 | 58.4 | 6.4 | 35.1 | 76.0 | 24.0 |
| PPID | rs7689418 | G/T | 0 | 296 | 172 | 19 | 105 | 58.1 | 6.4 | 35.5 | 75.8 | 24.2 |
| PTPRC | rs1932433 | C/T | 2 | 294 | 104 | 52 | 138 | 35.4 | 17.7 | 46.9 | 58.8 | 41.2 |
| PTPRC | rs17670373 | A/G | 0 | 296 | 144 | 36 | 116 | 48.6 | 12.2 | 39.2 | 68.2 | 31.8 |
| SLC2A2 | rs7646014 | C/G | 0 | 296 | 6 | 219 | 71 | 2 | 74 | 24 | 14.0 | 86.0 |
| SLC2A2 | rs1604038 | C/T | 0 | 296 | 138 | 26 | 132 | 46.6 | 8.8 | 44.6 | 68.9 | 31.1 |
| SLC2A2 | rs5400 | C/T | 0 | 296 | 220 | 6 | 70 | 74.3 | 2 | 23.6 | 86.1 | 13.9 |
| SLC2A2 | rs11721319 | A/G | 0 | 296 | 6 | 221 | 69 | 2 | 74.7 | 23.3 | 13.7 | 86.3 |
| SPOCK | rs1229729 | A/G | 0 | 296 | 93 | 72 | 131 | 31.4 | 24.3 | 44.3 | 53.5 | 46.5 |
| SPOCK | rs1229731 | A/G | 0 | 296 | 72 | 92 | 132 | 24.3 | 31.1 | 44.6 | 46.6 | 53.4 |
| SPOCK | rs2961633 | A/G | 1 | 295 | 58 | 97 | 140 | 19.7 | 32.9 | 47.5 | 43.4 | 56.6 |
| SPOCK | rs2961632 | C/T | 2 | 294 | 99 | 55 | 140 | 33.7 | 18.7 | 47.6 | 57.5 | 42.5 |
| SPOCK | rs12656717 | A/G | 0 | 296 | 56 | 87 | 153 | 18.9 | 29.4 | 51.7 | 44.8 | 55.2 |
| TGFBR2 | rs4955212 | C/T | 0 | 296 | 154 | 29 | 113 | 52 | 9.8 | 38.2 | 71.1 | 28.9 |
| TGFBR2 | rs1019855 | C/T | 1 | 295 | 1 | 238 | 56 | 0.3 | 80.7 | 19 | 9.8 | 90.2 |
| TGFBR2 | rs2082225 | A/G | 1 | 295 | 237 | 1 | 57 | 80.3 | 0.3 | 19.3 | 90.0 | 10.0 |
| TGFBR2 | rs9823731 | A/G | 0 | 296 | 50 | 106 | 140 | 16.9 | 35.8 | 47.3 | 40.5 | 59.5 |

TABLE III B
AMD Disease Population Cases

| | | | | | Allele Frequencies: | | | | | | | |
|---|---|---|---|---|---|---|---|---|---|---|---|---|
| | | Allele | Disease | | Disease Population | | | Allele Frequencies (percentages): Disease Population | | | | |
| | | 1/ | Undeter. | | Homozygotes | | | Homozygotes | | | Allele 1 | Allele 2 |
| Gene | SNP | Allele 2 | Frequency | Disease N | Allele 1 | Allele 2 | Heterozygotes | Allele 1 | Allele 2 | Heterozygotes | Overall | Overall |
| ADAM12 | rs1676717 | A/G | 0 | 505 | 68 | 208 | 229 | 13.5 | 41.2 | 45.3 | 36.1 | 63.9 |
| ADAM12 | rs1621212 | C/T | 0 | 505 | 206 | 68 | 231 | 40.8 | 13.5 | 45.7 | 63.7 | 36.3 |
| ADAM12 | rs12779767 | C/T | 0 | 505 | 175 | 78 | 252 | 34.7 | 15.4 | 49.9 | 59.6 | 40.4 |
| ADAM12 | rs11244834 | C/T | 0 | 505 | 78 | 175 | 252 | 15.4 | 34.7 | 49.9 | 40.4 | 59.6 |
| ADAM19 | rs12189024 | A/G | 0 | 505 | 51 | 244 | 210 | 10.1 | 48.3 | 41.6 | 30.9 | 69.1 |
| ADAM19 | rs7725839 | A/C | 1 | 504 | 22 | 337 | 145 | 4.4 | 66.9 | 28.8 | 18.8 | 81.3 |
| ADAM19 | rs11740315 | A/G | 48 | 457 | 48 | 217 | 192 | 10.5 | 47.5 | 42.0 | 31.5 | 68.5 |
| ADAM19 | rs7719224 | C/T | 0 | 505 | 339 | 22 | 144 | 67.1 | 4.4 | 28.5 | 81.4 | 18.6 |
| ADAM19 | rs6878446 | A/G | 0 | 505 | 58 | 229 | 218 | 11.5 | 45.3 | 43.2 | 33.1 | 66.9 |
| APBA2 | rs3829467 | C/T | 3 | 502 | 9 | 396 | 97 | 1.8 | 78.9 | 19.3 | 11.5 | 88.5 |
| APOB | rs12714097 | C/T | 0 | 505 | 505 | 0 | 0 | 100.0 | 0.0 | 0.0 | 100.0 | 0.0 |
| BMP7 | rs6014959 | A/G | 1 | 504 | 382 | 8 | 114 | 75.8 | 1.6 | 22.6 | 87.1 | 12.9 |
| BMP7 | rs6064517 | C/T | 0 | 505 | 386 | 8 | 111 | 76.4 | 1.6 | 22.0 | 87.4 | 12.6 |
| BMP7 | rs162315 | A/G | 0 | 505 | 35 | 283 | 187 | 6.9 | 56.0 | 37.0 | 25.4 | 74.6 |
| BMP7 | rs162316 | A/G | 0 | 505 | 34 | 283 | 188 | 6.7 | 56.0 | 37.2 | 25.3 | 74.7 |
| C1Qa | rs172378 | A/G | 2 | 503 | 212 | 67 | 224 | 42.1 | 13.3 | 44.5 | 64.4 | 35.6 |
| C1RL | rs61917913 | A/G | 1 | 504 | 0 | 459 | 45 | 0.0 | 91.1 | 8.9 | 4.5 | 95.5 |
| C4BPA | rs2842706 | A/G | 32 | 473 | 473 | 0 | 0 | 100.0 | 0.0 | 0.0 | 100.0 | 0.0 |
| C4BPA | rs1126618 | C/T | 1 | 504 | 360 | 11 | 133 | 71.4 | 2.2 | 26.4 | 84.6 | 15.4 |
| C5 | rs7033790 | C/T | 0 | 505 | 314 | 37 | 154 | 62.2 | 7.3 | 30.5 | 77.4 | 22.6 |
| C5 | rs10739585 | C/G | 0 | 505 | 314 | 37 | 154 | 62.2 | 7.3 | 30.5 | 77.4 | 22.6 |
| C5 | rs2230214 | A/G | 0 | 505 | 7 | 418 | 80 | 1.4 | 82.8 | 15.8 | 9.3 | 90.7 |
| C5 | rs10985127 | A/G | 4 | 501 | 350 | 16 | 135 | 69.9 | 3.2 | 26.9 | 83.3 | 16.7 |
| C5 | rs2300932 | A/C | 0 | 505 | 87 | 181 | 237 | 17.2 | 35.8 | 46.9 | 40.7 | 59.3 |
| C5 | rs12683026 | A/G | 0 | 505 | 427 | 4 | 74 | 84.6 | 0.8 | 14.7 | 91.9 | 8.1 |
| C5 | rs4837805 | A/G | 0 | 505 | 188 | 80 | 237 | 37.2 | 15.8 | 46.9 | 60.7 | 39.3 |
| C8A | MRD_4048 | C/G | 1 | 504 | 491 | 0 | 13 | 97.4 | 0.0 | 2.6 | 98.7 | 1.3 |
| C8A | MRD_4044 | A/C | 0 | 505 | 0 | 492 | 13 | 0.0 | 97.4 | 2.6 | 1.3 | 98.7 |
| CCL28 | rs7380703 | G/T | 1 | 504 | 51 | 253 | 200 | 10.1 | 50.2 | 39.7 | 30.0 | 70.0 |
| CCL28 | rs11741246 | A/G | 1 | 504 | 113 | 159 | 232 | 22.4 | 31.5 | 46.0 | 45.4 | 54.6 |
| CCL28 | rs4443426 | C/T | 0 | 505 | 159 | 111 | 235 | 31.5 | 22.0 | 46.5 | 54.8 | 45.2 |
| CLU | MRD_4452 | A/G | 0 | 505 | 0 | 478 | 27 | 0.0 | 94.7 | 5.3 | 2.7 | 97.3 |
| COL9A1 | rs1135056 | A/G | 1 | 504 | 193 | 85 | 226 | 38.3 | 16.9 | 44.8 | 60.7 | 39.3 |
| FGFR2 | rs2981582 | C/T | 0 | 505 | 210 | 69 | 226 | 41.6 | 13.7 | 44.8 | 64.0 | 36.0 |
| FGFR2 | rs2912774 | A/C | 0 | 505 | 73 | 205 | 227 | 14.5 | 40.6 | 45.0 | 36.9 | 63.1 |
| FGFR2 | rs1319093 | A/T | 0 | 505 | 12 | 375 | 118 | 2.4 | 74.3 | 23.4 | 14.1 | 85.9 |
| FGFR2 | rs10510088 | A/G | 0 | 505 | 339 | 19 | 147 | 67.1 | 3.8 | 29.1 | 81.7 | 18.3 |
| FGFR2 | rs12412931 | A/G | 0 | 505 | 12 | 375 | 118 | 2.4 | 74.3 | 23.4 | 14.1 | 85.9 |
| HABP2 | rs3740532 | C/T | 1 | 504 | 21 | 296 | 187 | 4.2 | 58.7 | 37.1 | 22.7 | 77.3 |
| HABP2 | rs7080536 | A/G | 2 | 503 | 1 | 457 | 45 | 0.2 | 90.9 | 8.9 | 4.7 | 95.3 |
| EMID2 | rs17135580 | C/T | 0 | 505 | 12 | 358 | 135 | 2.4 | 70.9 | 26.7 | 15.7 | 84.3 |
| EMID2 | rs12536189 | C/T | 1 | 504 | 12 | 358 | 134 | 2.4 | 71.0 | 26.6 | 15.7 | 84.3 |
| EMID2 | rs7778986 | A/G | 15 | 490 | 13 | 334 | 143 | 2.7 | 68.2 | 29.2 | 17.2 | 82.8 |
| EMID2 | rs11766744 | A/G | 2 | 503 | 11 | 361 | 131 | 2.2 | 71.8 | 26.0 | 15.2 | 84.8 |
| COL6A3 | rs4663722 | C/G | 2 | 503 | 435 | 3 | 65 | 86.5 | 0.6 | 12.9 | 92.9 | 7.1 |
| COL6A3 | rs1874573 | A/G | 0 | 505 | 184 | 63 | 258 | 36.4 | 12.5 | 51.1 | 62.0 | 38.0 |
| COL6A3 | rs12992087 | C/T | 0 | 505 | 333 | 17 | 155 | 65.9 | 3.4 | 30.7 | 81.3 | 18.7 |
| IFNAR2 | rs2826552 | A/T | 30 | 475 | 59 | 168 | 248 | 12.4 | 35.4 | 52.2 | 38.5 | 61.5 |
| COL4A1 | rs7338606 | C/T | 0 | 505 | 344 | 18 | 143 | 68.1 | 3.6 | 28.3 | 82.3 | 17.7 |
| COL4A1 | rs11842143 | C/G | 0 | 505 | 70 | 207 | 228 | 13.9 | 41.0 | 45.1 | 36.4 | 63.6 |
| COL4A1 | rs595325 | G/T | 1 | 504 | 28 | 319 | 157 | 5.6 | 63.3 | 31.2 | 21.1 | 78.9 |
| COL4A1 | rs9301441 | C/T | 0 | 505 | 104 | 160 | 241 | 20.6 | 31.7 | 47.7 | 44.5 | 55.5 |
| COL4A1 | rs754880 | A/G | 0 | 505 | 109 | 149 | 247 | 21.6 | 29.5 | 48.9 | 46.0 | 54.0 |
| COL4A1 | rs7139492 | C/T | 0 | 505 | 297 | 30 | 178 | 58.8 | 5.9 | 35.2 | 76.4 | 23.6 |
| COL4A1 | rs72509 | G/T | 0 | 505 | 11 | 376 | 118 | 2.2 | 74.5 | 23.4 | 13.9 | 86.1 |
| FBLN2 | rs9843344 | A/G | 0 | 505 | 51 | 235 | 219 | 10.1 | 46.5 | 43.4 | 31.8 | 68.2 |
| FBLN2 | rs1562808 | C/T | 1 | 504 | 252 | 36 | 216 | 50.0 | 7.1 | 42.9 | 71.4 | 28.6 |
| FBN2 | rs10057855 | A/G | 0 | 505 | 7 | 384 | 114 | 1.4 | 76.0 | 22.6 | 12.7 | 87.3 |
| FBN2 | rs10057405 | A/C | 0 | 505 | 366 | 9 | 130 | 72.5 | 1.8 | 25.7 | 85.3 | 14.7 |
| FBN2 | rs331075 | A/G | 0 | 505 | 140 | 105 | 260 | 27.7 | 20.8 | 51.5 | 53.5 | 46.5 |
| FBN2 | rs17676236 | C/G | 0 | 505 | 8 | 367 | 130 | 1.6 | 72.7 | 25.7 | 14.5 | 85.5 |
| FBN2 | rs6891153 | C/T | 1 | 504 | 4 | 406 | 94 | 0.8 | 80.6 | 18.7 | 10.1 | 89.9 |
| FBN2 | rs17676260 | C/T | 0 | 505 | 8 | 366 | 131 | 1.6 | 72.5 | 25.9 | 14.6 | 85.4 |
| FBN2 | rs154001 | C/T | 0 | 505 | 69 | 205 | 231 | 13.7 | 40.6 | 45.7 | 36.5 | 63.5 |
| FBN2 | rs3805653 | C/T | 0 | 505 | 276 | 23 | 206 | 54.7 | 4.6 | 40.8 | 75.0 | 25.0 |
| FBN2 | rs3828661 | A/C | 0 | 505 | 277 | 24 | 204 | 54.9 | 4.8 | 40.4 | 75.0 | 25.0 |
| FBN2 | rs3828661 | A/C | 0 | 505 | 277 | 24 | 204 | 54.9 | 4.8 | 40.4 | 75.0 | 25.0 |
| FBN2 | rs11241955 | A/G | 0 | 505 | 39 | 252 | 214 | 7.7 | 49.9 | 42.4 | 28.9 | 71.1 |
| FBN2 | rs6882394 | C/T | 8 | 497 | 49 | 219 | 229 | 9.9 | 44.1 | 46.1 | 32.9 | 67.1 |
| FBN2 | rs432792 | C/T | 0 | 505 | 6 | 385 | 114 | 1.2 | 76.2 | 22.6 | 12.5 | 87.5 |
| FBN2 | rs13181926 | C/T | 0 | 505 | 285 | 29 | 191 | 56.4 | 5.7 | 37.8 | 75.3 | 24.7 |
| FCN1 | rs10117466 | G/T | 3 | 502 | 197 | 61 | 244 | 39.2 | 12.2 | 48.6 | 63.5 | 36.5 |
| FCN1 | rs7857015 | A/G | 0 | 505 | 186 | 67 | 252 | 36.8 | 13.3 | 49.9 | 61.8 | 38.2 |
| FCN1 | rs2989727 | C/T | 0 | 505 | 65 | 217 | 223 | 12.9 | 43.0 | 44.2 | 35.0 | 65.0 |
| FCN1 | rs3012788 | C/T | 34 | 471 | 286 | 6 | 179 | 60.7 | 1.3 | 38.0 | 79.7 | 20.3 |
| HS3ST4 | rs4441276 | A/G | 1 | 504 | 248 | 61 | 195 | 49.2 | 12.1 | 38.7 | 68.6 | 31.4 |
| HS3ST4 | rs12921387 | C/T | 2 | 503 | 58 | 230 | 215 | 11.5 | 45.7 | 42.7 | 32.9 | 67.1 |
| IGLC1 | rs1065464 | C/G | 3 | 502 | 0 | 366 | 136 | 0.0 | 72.9 | 27.1 | 13.5 | 86.5 |
| IGLC1 | rs4820495 | C/T | 0 | 505 | 214 | 57 | 234 | 42.4 | 11.3 | 46.3 | 65.5 | 34.5 |
| IL12RB1 | rs273493 | C/T | 12 | 493 | 428 | 0 | 65 | 86.8 | 0.0 | 13.2 | 93.4 | 6.6 |
| ITGAX | rs2230429 | C/G | 1 | 504 | 216 | 75 | 213 | 42.9 | 14.9 | 42.3 | 64.0 | 36.0 |
| ITGAX | rs11574630 | C/T | 0 | 505 | 216 | 70 | 219 | 42.8 | 13.9 | 43.4 | 64.5 | 35.5 |
| MASP1 | rs12638131 | G/T | 1 | 504 | 292 | 36 | 176 | 57.9 | 7.1 | 34.9 | 75.4 | 24.6 |
| MASP2 | rs12142107 | C/T | 2 | 503 | 492 | 0 | 11 | 97.8 | 0.0 | 2.2 | 98.9 | 1.1 |
| MYOC | rs2236875 | G/T | 0 | 505 | 434 | 1 | 70 | 85.9 | 0.2 | 13.9 | 92.9 | 7.1 |
| MYOC | rs12035960 | C/T | 0 | 505 | 434 | 1 | 70 | 85.9 | 0.2 | 13.9 | 92.9 | 7.1 |
| MYOC | rs235868 | A/G | 3 | 502 | 232 | 55 | 215 | 46.2 | 11.0 | 42.8 | 67.6 | 32.4 |
| PPID | rs8396 | A/G | 0 | 505 | 234 | 47 | 224 | 46.3 | 9.3 | 44.4 | 68.5 | 31.5 |
| PPID | rs7689418 | G/T | 1 | 504 | 235 | 46 | 223 | 46.6 | 9.1 | 44.2 | 68.8 | 31.3 |
| PTPRC | rs1932433 | C/T | 3 | 502 | 211 | 48 | 243 | 42.0 | 9.6 | 48.4 | 66.2 | 33.8 |
| PTPRC | rs17670373 | A/G | 0 | 505 | 188 | 62 | 255 | 37.2 | 12.3 | 50.5 | 62.5 | 37.5 |
| SLC2A2 | rs7646014 | C/G | 0 | 505 | 2 | 416 | 87 | 0.4 | 82.4 | 17.2 | 9.0 | 91.0 |
| SLC2A2 | rs1604038 | C/T | 1 | 504 | 286 | 31 | 187 | 56.7 | 6.2 | 37.1 | 75.3 | 24.7 |
| SLC2A2 | rs5400 | C/T | 0 | 505 | 413 | 3 | 89 | 81.8 | 0.6 | 17.6 | 90.6 | 9.4 |
| SLC2A2 | rs11721319 | A/G | 1 | 504 | 3 | 412 | 89 | 0.6 | 81.7 | 17.7 | 9.4 | 90.6 |
| SPOCK | rs1229729 | A/G | 0 | 505 | 169 | 75 | 261 | 33.5 | 14.9 | 51.7 | 59.3 | 40.7 |
| SPOCK | rs1229731 | A/G | 0 | 505 | 75 | 169 | 261 | 14.9 | 33.5 | 51.7 | 40.7 | 59.3 |
| SPOCK | rs2961633 | A/G | 5 | 500 | 58 | 187 | 255 | 11.6 | 37.4 | 51.0 | 37.1 | 62.9 |
| SPOCK | rs2961632 | C/T | 0 | 505 | 191 | 58 | 256 | 37.8 | 11.5 | 50.7 | 63.2 | 36.8 |
| SPOCK | rs12656717 | A/G | 0 | 505 | 126 | 111 | 268 | 25.0 | 22.0 | 53.1 | 51.5 | 48.5 |
| TGFBR2 | rs4955212 | C/T | 0 | 505 | 305 | 29 | 171 | 60.4 | 5.7 | 33.9 | 77.3 | 22.7 |
| TGFBR2 | rs1019855 | C/T | 0 | 505 | 9 | 377 | 119 | 1.8 | 74.7 | 23.6 | 13.6 | 86.4 |
| TGFBR2 | rs2082225 | A/G | 0 | 505 | 377 | 9 | 119 | 74.7 | 1.8 | 23.6 | 86.4 | 13.6 |
| TGFBR2 | rs9823731 | A/G | 0 | 505 | 66 | 213 | 226 | 13.1 | 42.2 | 44.8 | 35.4 | 64.6 |

TABLE III C
Differences in Allele Frequencies between AMD Control and
Disease Populations

| | | | Difference in | | Difference in | |
|---|---|---|---|---|---|---|
| | | | Percentage | Difference in | Percentage | |
| | | | Allele | Percentage | Allele | Difference in |
| | | Allele 1/ | Freqeuency | (Hetero- | Freqeuency | Percentage |
| Gene | SNP | Allele 2 | (Allele 1) | Both) | (Allele 2) | (Undetermined) |
| ADAM12 | rs1676717 | A/G | 4.1 | 8.1 | 12.2 | 2.0 |
| ADAM12 | rs1621212 | C/T | 11.1 | 7.3 | 3.7 | 0.0 |
| ADAM12 | rs12779767 | C/T | 7.2 | 2.6 | 4.6 | 0.0 |
| ADAM12 | rs11244834 | C/T | 4.6 | 2.1 | 6.7 | 0.3 |
| ADAM19 | rs12189024 | A/G | 3.7 | 7.1 | 10.8 | 0.0 |
| ADAM19 | rs7725839 | A/C | 2.4 | 6.2 | 8.4 | 0.2 |
| ADAM19 | rs11740315 | A/G | 2.4 | 8.3 | 10.6 | 7.4 |
| ADAM19 | rs7719224 | C/T | 7.8 | 5.4 | 2.4 | 0.3 |
| ADAM19 | rs6878446 | A/G | 2 | 6.7 | 8.8 | 0.0 |
| APBA2 | rs3829467 | C/T | 1.5 | 4.6 | 6 | 0.8 |
| APOB | rs12714097 | C/T | 1.4 | 1.4 | 0 | 0.0 |
| BMP7 | rs6014959 | A/G | 7.6 | 7.3 | 0.2 | 0.1 |
| BMP7 | rs6064517 | C/T | 7.4 | 6.8 | 0.6 | 0.0 |
| BMP7 | rs162315 | A/G | 1.8 | 6.6 | 8.5 | 0.0 |
| BMP7 | rs162316 | A/G | 1.6 | 6.8 | 8.5 | 0.0 |
| C1Qa | rs172378 | A/G | 7.2 | 1.9 | 5.3 | 0.1 |
| C1RL | rs61917913 | A/G | 0 | 3.8 | 3.8 | 0.2 |
| C4BPA | rs2842706 | A/G | 1.1 | 1.1 | 0 | 2.1 |
| C4BPA | rs1126618 | C/T | 7.9 | 7.7 | 0.2 | 0.2 |
| C5 | rs7033790 | C/T | 6.4 | 2.1 | 4.3 | 0.0 |
| C5 | rs10739585 | C/G | 6.4 | 2.1 | 4.3 | 0.0 |
| C5 | rs2230214 | A/G | 0.6 | 6.8 | 7.5 | 0.0 |
| C5 | rs10985127 | A/G | 8.6 | 7 | 1.6 | 0.6 |
| C5 | rs2300932 | A/C | 4.7 | 2.6 | 7.4 | 0.0 |
| C5 | rs12683026 | A/G | 6.2 | 5.2 | 0.9 | 0.0 |
| C5 | rs4837805 | A/G | 6 | 1.6 | 4.3 | 0.0 |
| C8A | MRD_4048 | C/G | 2.3 | 2.3 | 0 | 0.1 |
| C8A | MRD_4044 | A/C | 0 | 2.3 | 2.3 | 0.7 |
| CCL28 | rs7380703 | G/T | 6 | 6.6 | 12.6 | 0.2 |
| CCL28 | rs11741246 | A/G | 4.6 | 3.3 | 7.9 | 0.2 |
| CCL28 | rs4443426 | C/T | 7.2 | 2.1 | 5 | 0.0 |
| CLU | MRD_4452 | A/G | 0 | 3.3 | 3.3 | 0.0 |
| COL9A1 | rs1135056 | A/G | 9.9 | 9.3 | 0.7 | 0.2 |
| FGFR2 | rs2981582 | C/T | 9.8 | 3.8 | 5.9 | 0.0 |
| FGFR2 | rs2912774 | A/C | 6.1 | 2.3 | 8.5 | 0.0 |
| FGFR2 | rs1319093 | A/T | 0.3 | 7.2 | 7.6 | 0.7 |
| FGFR2 | rs10510088 | A/G | 8 | 7.4 | 0.6 | 0.0 |
| FGFR2 | rs12412931 | A/G | 0.3 | 7 | 7.4 | 0.0 |
| HABP2 | rs3740532 | C/T | 2.5 | 5.3 | 7.9 | 0.2 |
| HABP2 | rs7080536 | A/G | 0.2 | 4.1 | 4.3 | 0.3 |
| EMID2 | rs17135580 | C/T | 1.7 | 6.4 | 8.1 | 0.3 |
| EMID2 | rs12536189 | C/T | 1.7 | 6.3 | 8.1 | 0.2 |
| EMID2 | rs7778986 | A/G | 1.3 | 6.2 | 7.4 | 1.3 |
| EMID2 | rs11766744 | A/G | 0.5 | 6.3 | 6.8 | 0.1 |
| COL6A3 | rs4663722 | C/G | 5.1 | 3.7 | 1.4 | 0.4 |
| COL6A3 | rs1874573 | A/G | 11.6 | 8.9 | 2.7 | 0.0 |
| COL6A3 | rs12992087 | C/T | 3 | 0 | 3.1 | 0.0 |
| IFNAR2 | rs2826552 | A/T | 1.3 | 10.1 | 11.3 | 5.9 |
| COL4A1 | rs7338606 | C/T | 11.3 | 9.2 | 2.1 | 0.0 |
| COL4A1 | rs11842143 | C/G | 4.4 | 6.6 | 11 | 0.0 |
| COL4A1 | rs595325 | G/T | 1.2 | 7.9 | 9 | 0.2 |
| COL4A1 | rs9301441 | C/T | 4.4 | 4.5 | 8.8 | 0.0 |
| COL4A1 | rs754880 | A/G | 6.7 | 1.8 | 5 | 0.0 |
| COL4A1 | rs7139492 | C/T | 8.5 | 5.9 | 2.7 | 1.4 |
| COL4A1 | rs72509 | G/T | 1.2 | 5.3 | 6.6 | 0.0 |
| FBLN2 | rs9843344 | A/G | 3.8 | 5.2 | 9 | 0.0 |
| FBLN2 | rs1562808 | C/T | 8.2 | 5.1 | 3.1 | 0.5 |
| FBN2 | rs10057855 | A/G | 0.3 | 10.1 | 9.8 | 0.0 |
| FBN2 | rs10057405 | A/C | 9.9 | 9.8 | 0.1 | 0.0 |
| FBN2 | rs331075 | A/G | 8.8 | 1.2 | 7.6 | 0.0 |
| FBN2 | rs17676236 | C/G | 0.4 | 9.1 | 8.7 | 0.0 |
| FBN2 | rs6891153 | C/T | 0.6 | 7.9 | 7.2 | 0.2 |
| FBN2 | rs17676260 | C/T | 0.4 | 9 | 8.6 | 0.0 |
| FBN2 | rs154001 | C/T | 2.9 | 7.9 | 10.8 | 0.0 |
| FBN2 | rs3805653 | C/T | 8.5 | 7 | 1.6 | 0.0 |
| FBN2 | rs3828661 | A/C | 8.2 | 6.6 | 1.7 | 1.0 |
| FBN2 | rs3828661 | A/C | 8.2 | 6.6 | 1.7 | 1.0 |
| FBN2 | rs11241955 | A/G | 3.1 | 4.2 | 7.3 | 0.0 |
| FBN2 | rs6882394 | C/T | 3.3 | 3 | 6.2 | 1.1 |
| FBN2 | rs432792 | C/T | 0.5 | 6.1 | 6.6 | 0.0 |
| FBN2 | rs13181926 | C/T | 6.1 | 3.7 | 2.3 | 0.0 |
| FCN1 | rs10117466 | G/T | 11 | 7.6 | 3.4 | 0.3 |
| FCN1 | rs7857015 | A/G | 9.5 | 5.3 | 4.2 | 0.0 |
| FCN1 | rs2989727 | C/T | 5 | 2.1 | 7.2 | 0.0 |
| FCN1 | rs3012788 | C/T | 8.2 | 7.7 | 0.6 | 3.1 |
| HS3ST4 | rs4441276 | A/G | 6 | 11 | 5 | 0.2 |
| HS3ST4 | rs12921387 | C/T | 4.7 | 0.7 | 5.5 | 0.1 |
| IGLC1 | rs1065464 | C/G | 1.4 | 6.2 | 4.8 | 0.6 |
| IGLC1 | rs4820495 | C/T | 8.3 | 6.8 | 1.5 | 0.0 |
| IL12RB1 | rs273493 | C/T | 5.8 | 5.8 | 0 | 2.0 |
| ITGAX | rs2230429 | C/G | 4.9 | 1.8 | 6.8 | 0.1 |
| ITGAX | rs11574630 | C/T | 6.2 | 0.2 | 6.1 | 0.0 |
| MASP1 | rs12638131 | G/T | 8.6 | 7.3 | 1.3 | 0.2 |
| MASP2 | rs12142107 | C/T | 2.9 | 2.9 | 0 | 0.6 |
| MYOC | rs2236875 | G/T | 6.2 | 4.3 | 1.8 | 0.0 |
| MYOC | rs12035960 | C/T | 5.8 | 4 | 1.8 | 0.0 |
| MYOC | rs235868 | A/G | 5.3 | 1.1 | 4.2 | 0.3 |
| PPID | rs8396 | A/G | 12.1 | 9.3 | 2.9 | 0.0 |
| PPID | rs7689418 | G/T | 11.5 | 8.7 | 2.7 | 0.2 |
| PTPRC | rs1932433 | C/T | 6.6 | 1.5 | 8.1 | 0.1 |
| PTPRC | rs17670373 | A/G | 11.4 | 11.3 | 0.1 | 0.0 |
| SLC2A2 | rs7646014 | C/G | 1.6 | 6.8 | 8.4 | 0.0 |
| SLC2A2 | rs1604038 | C/T | 10.1 | 7.5 | 2.6 | 0.2 |
| SLC2A2 | rs5400 | C/T | 7.5 | 6 | 1.4 | 0.0 |
| SLC2A2 | rs11721319 | A/G | 1.4 | 5.6 | 7 | 0.2 |
| SPOCK | rs1229729 | A/G | 2.1 | 7.4 | 9.4 | 0.0 |
| SPOCK | rs1229731 | A/G | 9.4 | 7.1 | 2.4 | 0.0 |
| SPOCK | rs2961633 | A/G | 8.1 | 3.5 | 4.5 | 0.7 |
| SPOCK | rs2961632 | C/T | 4.1 | 3.1 | 7.2 | 0.7 |
| SPOCK | rs12656717 | A/G | 6.1 | 1.4 | 7.4 | 0.0 |
| TGFBR2 | rs4955212 | C/T | 8.4 | 4.3 | 4.1 | 0.0 |
| TGFBR2 | rs1019855 | C/T | 1.5 | 4.6 | 6 | 0.3 |
| TGFBR2 | rs2082225 | A/G | 5.6 | 4.3 | 1.5 | 0.3 |
| TGFBR2 | rs9823731 | A/G | 3.8 | 2.5 | 6.4 | 0.0 |

TABLE IV A
Additional AMD Control Population Cases

| | | | | | Allele Frequencies: | | | | | | | |
|---|---|---|---|---|---|---|---|---|---|---|---|---|
| | | Allele | Control | | Control Population | | | Allele Frequencies (percentages): Control Population | | | | |
| | | 1/ | Undeter. | | Homozygotes | | | Homozygotes | | | Allele 1 | Allele 2 |
| Gene | SNP | Allele 2 | Frequency | Control N | Allele 1 | Allele 2 | Heterozygotes | Allele 1 | Allele 2 | Heterozygotes | Overall | Overall |
| C3 | rs2547438 | G/T | 27 | 269 | 18 | 141 | 110 | 6.7 | 52.4 | 40.9 | 27.1 | 72.9 |
| C3 | rs2230199 | C/G | 1 | 295 | 18 | 187 | 90 | 6.1 | 63.4 | 30.5 | 21.4 | 78.6 |
| C3 | rs1047286 | A/G | 12 | 284 | 14 | 182 | 88 | 4.9 | 64.1 | 31 | 20.4 | 79.6 |
| C3 | rs3745567 | A/G | 3 | 293 | 4 | 223 | 66 | 1.4 | 76.1 | 22.5 | 12.6 | 87.4 |
| C3 | rs11569507 | A/G | 0 | 296 | 227 | 4 | 65 | 76.7 | 1.4 | 22 | 87.7 | 12.3 |
| C3 | rs11085197 | C/G | 0 | 296 | 17 | 187 | 92 | 5.7 | 63.2 | 31.1 | 21.3 | 78.7 |
| C7 | rs2271708 | A/G | 0 | 296 | 295 | 0 | 1 | 99.7 | 0 | 0.3 | 99.8 | 0.2 |
| C7 | rs1055021 | A/C | 0 | 296 | 0 | 243 | 53 | 0 | 82.1 | 17.9 | 9.0 | 91.0 |
| C9 | rs476569 | C/T | 0 | 296 | 70 | 74 | 152 | 23.6 | 25 | 51.4 | 49.3 | 50.7 |
| C1NH | rs4926 | A/G | 0 | 296 | 14 | 168 | 114 | 4.7 | 56.8 | 38.5 | 24.0 | 76.0 |
| C1NH | rs2511988 | A/G | 0 | 296 | 30 | 128 | 138 | 10.1 | 43.2 | 46.6 | 33.4 | 66.6 |
| ITGA4 | rs3770115 | C/T | 0 | 296 | 119 | 37 | 140 | 40.2 | 12.5 | 47.3 | 63.9 | 36.1 |
| ITGA4 | rs4667319 | A/G | 0 | 296 | 115 | 42 | 139 | 38.9 | 14.2 | 47 | 62.3 | 37.7 |

TABLE IV B
Additional AMD Disease Population Cases

| | | | | | Allele Frequencies: | | | | | | | |
|---|---|---|---|---|---|---|---|---|---|---|---|---|
| | | | Disease | | Disease Population | | | Allele Frequencies (percentages): Disease Population | | | | |
| | | Allele 1/ | Undeter. | | Homozygotes | | | Homozygotes | | | Allele 1 | Allele 2 |
| Gene | SNP | Allele 2 | Frequency | Disease N | Allele 1 | Allele 2 | Heterozygotes | Allele 1 | Allele 2 | Heterozygotes | Overall | Overall |
| C3 | rs2230199 | G/T | 38 | 467 | 15 | 291 | 161 | 3.2 | 62.3 | 34.5 | 20.4 | 79.6 |
| C3 | rs1047286 | C/G | 1 | 504 | 43 | 281 | 180 | 8.5 | 55.8 | 35.7 | 26.4 | 73.6 |
| C3 | rs3745567 | A/G | 18 | 487 | 40 | 280 | 167 | 8.2 | 57.5 | 34.3 | 25.4 | 74.6 |
| C3 | rs11569507 | A/G | 0 | 505 | 0 | 410 | 95 | 0.0 | 81.2 | 18.8 | 9.4 | 90.6 |
| C3 | rs11085197 | A/G | 0 | 505 | 411 | 0 | 94 | 81.4 | 0.0 | 18.6 | 90.7 | 9.3 |
| C7 | rs2271708 | C/G | 0 | 505 | 41 | 286 | 178 | 8.1 | 56.6 | 35.2 | 25.7 | 74.3 |
| C7 | rs1055021 | A/G | 1 | 504 | 491 | 0 | 13 | 97.4 | 0.0 | 2.6 | 98.7 | 1.3 |
| C7 | rs1055021 | A/C | 0 | 505 | 10 | 412 | 83 | 2.0 | 81.6 | 16.4 | 10.2 | 89.8 |
| C9 | rs476569 | C/T | 0 | 505 | 161 | 97 | 247 | 31.9 | 19.2 | 48.9 | 56.3 | 43.7 |
| C1NH | rs4926 | A/G | 0 | 505 | 41 | 232 | 232 | 8.1 | 45.9 | 45.9 | 31.1 | 68.9 |
| C1NH | rs2511988 | A/G | 1 | 504 | 32 | 258 | 214 | 6.3 | 51.2 | 42.5 | 27.6 | 72.4 |
| ITGA4 | rs3770115 | C/T | 0 | 505 | 262 | 49 | 194 | 51.9 | 9.7 | 38.4 | 71.1 | 28.9 |
| ITGA4 | rs4667319 | A/G | 0 | 505 | 243 | 59 | 203 | 48.1 | 11.7 | 40.2 | 68.2 | 31.8 |

TABLE IV C
Differences in Allele Frequencies between Additional AMD Control
and Disease Populations

| | | | Difference in | | Difference in | |
|---|---|---|---|---|---|---|
| | | | Percentage | | Percentage | |
| | | | Allele | Difference in | Allele | Difference in |
| | | Allele 1/ | Freqeuency | Percentage | Freqeuency | Percentage |
| Gene | SNP | Allele 2 | (Allele 1) | (Hetero-Both) | (Allele 2) | (Undetermined) |
| C3 | rs2547438 | G/T | 3.5 | 6.4 | 9.9 | 1.6 |
| C3 | rs2230199 | C/G | 2.4 | 5.2 | 7.6 | 0.1 |
| C3 | rs1047286 | A/G | 3.3 | 3.3 | 6.6 | 0.5 |
| C3 | rs3745567 | A/G | 1.4 | 3.7 | 5.1 | 1.0 |
| C3 | rs11569507 | A/G | 4.7 | 3.4 | 1.4 | 0.0 |
| C3 | rs11085197 | C/G | 2.4 | 4.1 | 6.6 | 0.0 |
| C7 | rs2271708 | A/G | 2.3 | 2.3 | 0 | 0.2 |
| C7 | rs1055021 | A/C | 2 | 1.5 | 0.5 | 0.0 |
| C9 | rs476569 | C/T | 8.3 | 2.5 | 5.8 | 0.0 |
| C1NH | rs4926 | A/G | 3.4 | 7.4 | 10.9 | 0.0 |
| C1NH | rs2511988 | A/G | 3.8 | 4.1 | 8 | 0.2 |
| ITGA4 | rs3770115 | C/T | 11.7 | 8.9 | 2.8 | 0.0 |
| ITGA4 | rs4667319 | A/G | 9.2 | 6.8 | 2.5 | 0.0 |

TABLE V

| Gene Name | Gene Identifier |
|---|---|
| ADAM12 | ENSG00000148848 |
| ADAM19 | ENSG00000135074 |
| APBA2 | ENSG00000034053 |
| APOB | ENSG00000084674 |
| BMP7 | ENSG00000101144 |
| C1Qa | ENSG00000173372 |
| C1RL | ENSG00000139178 |
| C4BPA | ENSG00000123838 |
| C5 | ENSG00000106804 |
| C8A | ENSG00000157131 |
| CCL28 | ENSG00000151882 |
| CLU | ENSG00000120885 |
| COL9A1 | ENSG00000112280 |
| FGFR2 | ENSG00000066468 |
| HABP2 | ENSG00000148702 |
| EMID2 | ENSG00000160963 |
| COL6A3 | ENSG00000163359 |
| IFNAR2 | ENSG00000159110 |
| COL4A1 | ENSG00000187498 |
| FBLN2 | ENSG00000163520 |
| FBN2 | ENSG00000138829 |
| FCN1 | ENSG00000085265 |
| HS3ST4 | ENSG00000182601 |
| IGLC1 | ENSG00000211675 |
| IL12RB1 | ENSG00000096996 |
| ITGAX | ENSG00000140678 |
| MASP1 | ENSG00000127241 |
| MASP2 | ENSG00000009724 |
| MYOC | ENSG00000034971 |
| PPID | ENSG00000171497 |
| PTPRC | ENSG00000081237 |
| SLC2A2 | ENSG00000163581 |
| SPOCK | ENSG00000152377 |
| TGFBR2 | ENSG00000163513 |
| C3 | ENSG00000125730 |
| C7 | ENSG00000112936 |
| C9 | ENSG00000113600 |
| C1NH | ENSG00000149131 |
| ITGA4 | ENSG00000115232 |

TABLE VI
Risk-informative SNPs in the RCA locus

| | | | Allele Frequencies (percentages): Control Population | | | | |
|---|---|---|---|---|---|---|---|
| | | | Homozygotes | | | | Allele 2 |
| Gene | SNP | Allele 1/Allele 2 | Allele 1 | Allele 2 | Heterozygotes | Allele 1 Overall | Overall |
| F13B | rs5997 | A/G | 1 | 77.9 | 21 | 11.6 | 88.4 |
| F13B | rs6428380 | A/G | 1 | 78.4 | 20.6 | 11.3 | 88.7 |
| F13B | rs1412631 | C/T | 78.4 | 1 | 20.6 | 88.7 | 11.3 |
| F13B | rs1794006 | C/T | 78.4 | 1 | 20.6 | 88.7 | 11.3 |
| F13B | rs10801586 | C/T | 69.6 | 2 | 28.4 | 83.8 | 16.2 |
| F13B | rs2990510 | G/T | 8.4 | 45.6 | 45.9 | 31.4 | 68.6 |
| FHR1 | rs12027476 | C/G | 0 | 63.6 | 36.4 | 18.2 | 81.8 |
| FHR1 | rs436719 | A/C | 46.6 | 0 | 53.4 | 73.3 | 26.7 |
| FHR2 | rs12066959 | A/G | 5.5 | 58.7 | 35.8 | 23.4 | 76.6 |
| FHR2 | rs3828032 | A/G | 8.2 | 46.3 | 45.6 | 31.0 | 69.0 |
| FHR2 | rs6674522 | C/G | 1.4 | 76.7 | 22 | 12.3 | 87.7 |
| FHR2 | rs432366 | C/G | 0 | 47 | 53 | 26.5 | 73.5 |
| FHR4 | rs1409153 | A/G | 36.1 | 14.9 | 49 | 60.6 | 39.4 |
| FHR5 | rs10922153 | G/T | 23.6 | 25.7 | 50.7 | 49.0 | 51.0 |
| FHR5 | MRD_3905 | A/G | 3 | 57.8 | 39.2 | 22.6 | 77.4 |
| FHR5 | MRD_3906 | C/T | 57.8 | 3.7 | 38.5 | 77.0 | 23.0 |

| | | | | | | | | Frequencies |
|---|---|---|---|---|---|---|---|---|
| | | Allele Frequencies | | | | | Genotype- | Chi Square |
| | | (percentages): Disease Population | | | | | Likelihood | (both |
| | | Homozygotes | | | Allele 1 | Allele 2 | Ratio (3 | collapsed-2 |
| Gene | SNP | Allele 1 | Allele 2 | Heterozygotes | Overall | Overall | categories) | categories) |
| F13B | rs5997 | 0.4 | 90.1 | 9.5 | 5.2 | 94.8 | 2.48E−05 | 3.37E−06 |
| F13B | rs6428380 | 0.4 | 90.1 | 9.5 | 5.2 | 94.8 | 4.11E−05 | 5.81E−06 |
| F13B | rs1412631 | 90.1 | 0.4 | 9.5 | 94.8 | 5.2 | 4.11E−05 | 5.81E−06 |
| F13B | rs1794006 | 89.9 | 0.4 | 9.7 | 94.7 | 5.3 | 6.13E−05 | 8.87E−06 |
| F13B | rs10801586 | 82.2 | 1.4 | 16.4 | 90.4 | 9.6 | 2.43E−04 | 8.70E−05 |
| F13B | rs2990510 | 15.0 | 39.2 | 45.7 | 37.9 | 62.1 | 1.31E−02 | 8.67E−03 |
| FHR1 | rs12027476 | 0.0 | 78.2 | 21.8 | 10.9 | 89.1 | 1.24E−05 | 4.99E−05 |
| FHR1 | rs436719 | 58.8 | 0.0 | 41.2 | 79.4 | 20.6 | 8.32E−04 | 5.04E−03 |
| FHR2 | rs12066959 | 2.0 | 75.0 | 23.0 | 13.5 | 86.5 | 4.83E−06 | 4.38E−07 |
| FHR2 | rs3828032 | 5.0 | 62.7 | 32.3 | 21.1 | 78.9 | 3.29E−05 | 1.16E−05 |
| FHR2 | rs6674522 | 0.4 | 87.9 | 11.7 | 6.2 | 93.8 | 1.79E−04 | 2.40E−05 |
| FHR2 | rs432366 | 0.0 | 58.8 | 41.2 | 20.6 | 79.4 | 1.15E−03 | 6.34E−03 |
| FHR4 | rs1409153 | 17.0 | 36.8 | 46.1 | 40.1 | 59.9 | 3.25E−14 | 1.93E−15 |
| FHR5 | rs10922153 | 44.6 | 9.5 | 45.9 | 67.5 | 32.5 | 1.38E−12 | 2.27E−13 |
| FHR5 | MRD_3905 | 3.4 | 68.9 | 27.7 | 17.2 | 82.8 | 3.74E−03 | 8.03E−03 |
| FHR5 | MRD_3906 | 68.5 | 3.4 | 28.1 | 82.6 | 17.4 | 8.16E−03 | 6.81E−03 |

TABLE VII
Flanking Sequences for SNPs shown in Table I

| Gene | SNP | SNP Flanking Sequence |
|---|---|---|
| ADAM12 | rs1676717 | TCTTTAAAATGCTCTGTGCCTCTTAAGCAGNATTTATATGCTGAGGAATATATTTTAGTCA |
| ADAM12 | rs1621212 | GCTGGATTTTATTTTAAATTCTAAGCAGATNATGTTTTCATTTTTACAAAGAGATTCCATC |
| ADAM12 | rs12779767 | ggtgtgtatgtgtgtgtatgtgggcacgtgNgtatatttgtgtgtgtgcatgtgcatgggt |
| ADAM12 | rs11244834 | ACCCACTCTGCTGTAAGCTCTATTTTCCACNTGCTATTTTCTTCCACACTGACCCATTGCT |
| ADAM19 | rs12189024 | GCCAAGGGCAACTTGCCTTTATATATCCACNTTACATGTAAATTCGCTTTGACTCAGTTGG |
| ADAM19 | rs7725839 | CAAGAGGAGAGGAACAAAAATGACTGTGATNCCCATCTTTCTGGCTTCCCGAGGCCACCAT |
| ADAM19 | rs11740315 | GGTCACTGTTTTTCACCTGCACTCAATAGANAAAAGAATGTGTGCTTCTCACGGATGTCAT |
| ADAM19 | rs7719224 | AGAGAATTTAAACTATAGGAGAGATTTGTTNAGGGCGCTGCAGAACTCAAATACTGGCGGC |
| ADAM19 | rs6878446 | CAGTGGTTGATAAGCACAAAAACTATGTTCNACATCGCTAATAAACCACCTCCCCACGTCC |
| APBA2 | rs3829467 | AGGAGTGGAAGACACCCTCTGGTCCCCCTGNGCCCCCATGCCAGGCTCATGGGCTCTCTGG |
| APOB | rs12714097 | ATATTTGTCACAAACTCCACAGACACGGAGNGTTTTGCCACCAGTTCAGCCTGCATCTATA |
| BMP7 | rs6014959 | ctgaggctcagggaggccgggtaactttcaNaggtcacaaatcaggtgagcggctgaactc |
| BMP7 | rs6064517 | AATTGCATGGTTGTCCTTTAAACCTCTTTCNGGTGTGGGAAGCAGGAGAATATGAGATCAA |
| BMP7 | rs162315 | GCTTAAACAGAACCTGCAGTATTCAGAGCCNTGGGACAGTATTTAACACCCTAAAAATTAG |
| BMP7 | rs162316 | GGCTGGTCTGTGAGAAAGAAGCTTCCAAGCACNGAGGACTGAGGTTGGAATTGAGGAGAGAGA |
| C1Qa | rs172376 | AAAGCATTCTTCAAATTGTGAAGTGCTACANAAACATGAGCTATGCTGCAAGCCATGTACA |
| C1RL | rs61917913 | cgttgatggcctcagagcccctgctggcctcNctgatgggctgactatagttcacagctatag |
| C4BPA | rs2842706 | ACCTTCCATTAGAATTTGCATGAATTTTAGNTTTATCATGATGCCTTCCCTGAGTATTTCT |
| C4BPA | rs1126618 | tttcttgcactgtggagaatgaaacaataggNgtttggagaccaagccctcctacctgtgaaa |
| C5 | rs7033790 | TTGAGGGCAGGCATTTTTGTCTTATTTAGCNTTACATCCCAGTGTCTAGCATAGAATTTTG |
| C5 | rs10739585 | TTTGTAAGTAAATGGGGGAGGCCTTTATATNAGTTCTCAGTTGTTATGTGTACAGTTGAGG |
| C5 | rs2230214 | AGTGGGAGAACATCTGAATATTATTGTTACNCCCAAAAGCCCATATATTGACAAAATAACT |
| C5 | rs10985127 | ttaacctgcacctgtttgtcaaaacaatccaNatctatttcaacagctcatcacttattttaa |
| C5 | rs2300932 | TCGCACATAGTTGTGTGTAGTAAATGCATANTGAAGTCAAATAAAATTAAATGGAATAGCA |
| C5 | rs12683026 | tcagttaaaaacataagtttatataacctgaNttccctggatcaacttttctggaccactttt |
| C5 | rs4837805 | TCTACCTTTCCATCCCAACCACACcaaaatNgtgggagaatgtttacattatcctgtactt |
| C8A | MRD_4048 | agcttcgatatgactccacctgtgaacgtctNtactatggagatgatgagaaatactttcgga |
| C8A | MRD_4044 | aggagagtaagacgggcagctacacccgcagNagttacctgccagctgagcaactggtcagag |
| CCL28 | rs7380703 | tttgtttgtttttttcagatggagtctcacNttgtcagtcagactggagtgcagtagcacg |
| CCL28 | rs11741246 | gggggcaagtggactgagtccagaaagagcNtcagcaaagggagatggggtggggtagttt |
| CCL28 | rs4443426 | GATTCAGGATGCAAGGGTGGGAGTGGAGCANGTGCCCACAATCCACAGTGTGTTCTGTGGC |
| CLU | MRD_4452 | gcgtggtcaggggctgagttttccagttcagNatcaggactatggaggcacaacatggaggcc |
| COL9A1 | rs1135056 | CTCCAGGAGAGGTGGGACCCCGAGGACCCCNGGGGCTTCCTGTGAGTATTCCTTGCTGTTC |
| FGFR2 | rs2981582 | GCACTCATCGCCACTTAATGAACCTGTTTGNGGAGAGTCCACCTGGTGCCTGCCTGGCTTT |
| FGFR2 | rs2912774 | GGAAATTGATTTTTGGGTGCCTGGCTGTTANGCTAGGTAGGAAATATAGCTGGTGTGCTAC |
| FGFR2 | rs1319093 | CCCACCTCCCAGGGCTTTTAGGAGTGCAATNTGATGTGATAATAGGAAGATCTAGCACAGT |
| FGFR2 | rs10510088 | TGAGTGTGTATTCTGTGCCTTTTCATTCCGNGCTTTAAACACATCATCTATGTCGTTGATC |
| FGFR2 | rs12412931 | GAGGTGGCTTGAAGCCAGTAATATGCTCTTNGATGGAAACAGCTTTTTACTTTCACTCAGG |
| HABP2 | rs3740532 | TTTTCTCATCTTTGAACAGCAGGAAGAGGANTGGGACCTAGCACGTCTACAGGGTCCTACA |
| HABP2 | rs7080536 | ATGGGATAGTGAGCTGGGGCCTGGAGTGTGNGAAGAGGCCAGGGGTCTACACCCAAGTTAC |
| EMID2 | rs17135580 | ACCAAGCAGGTAGCTTCCGTGTGAGCGCAGNATTCCCCAGAGATGTGGATGGATCTCCTTC |
| EMID2 | rs12536189 | GGCAACTGCCTCCTGCCAGGAAAGGTGTGANCCTGAGTCTGACCCTGAGACTCAAGGAGTC |
| EMID2 | rs7778986 | AGAAGGCTTTGACTTGGGAAAGCATAACCCNCTGGACTCGGTTTCAGGGCTGGGTCTCCTG |
| EMID2 | rs11766744 | CGTGGCTTCCAAACCCTCCCCCTGGCGAAANGCAGCCTGAAGGAGCTGCTGCGGTTTAAGA |
| COL6A3 | rs4663722 | TCTTCTTCAAGAGGTATATGATGTTGGCCANCCACGCTTAGGTTCCCATCACACTGATGAC |
| COL6A3 | rs1874573 | CAGTCACCTCACCTCCTACCTCCTGTCCCANTGGCCATCTTGGTGGTAACCATTTTTAAAA |
| COL6A3 | rs12992087 | CCAGAACATTGTCTGCTGCGAAGAAGACAANCTTTGATTACAACCCCATGCTCCAAGCAGC |
| IFNAR2 | rs2826552 | acaacaacaaACTCACAGATACGTAGGTAANAAAGATAATACTTGGTAACAATGAATGGTA |
| COL4A1 | rs7338606 | TCTGTTTACAGAATGCCTTTTTAACTGAACNGAAGAATACCAGCTGCTATGCTACTCgtgt |
| COL4A1 | rs11842143 | AGGGCAGTGCTAGGCTAACTGGCACAGTGANCGTGCActggacatgggatataaaattgca |
| COL4A1 | rs595325 | ataaacgctggcatgctttccacttgagaNtcatcaattgcttacattttgccacattcg |
| COL4A1 | rs9301441 | TGAGCACTCTGATGCCTGTCGTGCCAGGCTNAGGAGCTTGGACCTGAATGCAAAAGATGGC |
| COL4A1 | rs754880 | ACTTCGTGCCTTctggtaatgttctggtccNtagtctgggttcactgctatgttcactttg |
| COL4A1 | rs7139492 | TGCATTAAAATGAGCAAGCTGCGtattattNttattattattttaaaattttGGCATATGg |
| COL4A1 | rs72509 | AGTGCGTCCCCAGCATCCGGGTCCCACGGANCCCCTCCCAAACCAAGTGTCAGCTCCAGGC |
| FBLN2 | rs9843344 | GCGCGCGGCTACCACGCCAGCGATGATGGGNCCAAGTGTGTGGGTAAGGCCAGCCGCCTCC |
| FBLN2 | rs1562808 | TATTAACCCAGGCAGGTGAGATGCGAGGCTNATGCTAGgatcaagagtgtggcctttgcag |
| FBN2 | rs10057855 | caaatcccagtctgtcatctactgagaacaNggccttgggaaaatcatttagtctctctgt |
| FBN2 | rs10057405 | GCAACTCCAGCCCTTAGACAGAAGGCACTCNAACTTTACCAAATGAATTATTTTGAATGGA |
| FBN2 | rs331075 | accactgatgggcatttaggctgtttccacNtctttatattgtgaacagtgctgtgatgaa |
| FBN2 | rs17676236 | CCTCACCTCGATAGAAACTAGCTTTGCAATNAACTATGTTACAACTCTGGGCCTTACTTTC |
| FBN2 | rs6891153 | TGGGATTTTGATATGAATGTTAGTGAAAAANAACATTTTTGATGCTATCATCCAGTCTAGT |
| FBN2 | rs17676260 | CTTTTCTTACTCCATGCAGCTAATTTCTGANTGGCAAAGAACAGCACTAAGATCAGTCACA |
| FBN2 | rs154001 | TACTGTTGACACAGCGTCCATTTGGACAAANGCCAGGGAACACCTCACACTCATTAACATC |
| FBN2 | rs3805653 | AAATGGTACCCTCATTCACTGAGACACATANATGTGCAATTTTTATGAAGAATGTTAATCT |
| FBN2 | rs3828661 | GATCTTACTTATTACATTCTTATTATAAACNAGAAATCTGGCATACTAACTTTTCCAGACC |
| FBN2 | rs11241955 | TTGCATGATAGTCTAAGAAAAACAATCAAGNTAACTTTCAGTAAGTTACATCACATCAAAA |
| FBN2 | rs6882394 | CATTCACAGGCTACACTTCTGAGAAAGGATNCTTGAAACAAAAGAGCCATGTGATAAGATA |
| FBN2 | rs432792 | TCCTGTTTATGTACACTGCTGTTTTTTCTANAGAGGATTTCAACTCTCCTTATTGATTAAT |
| FBN2 | rs13181926 | ATCAATCTATCTTTTTATGAATACTGTGTTNGTGATAGACTTCTGTCtttcttttctggtt |
| FCN1 | rs10117466 | ggctgcttagttgcaccaacaggaggtatgaNgtcatctcaaaggatgtttccttccactagg |
| FCN1 | rs7857015 | agatttgctcgaaggcaccgtcaagctagctNtgtagtaaaggattactttgccaagctctca |
| FCN1 | rs2989727 | tttttcaaatgaaatgttaaaacacaatcaaNgataaccaggctcatgggaataaaagacaat |
| FCN1 | rs3012788 | gccacggaaaggctggtccagagtgggctccNggcctggacactgcatctccccagccctgca |
| HS3ST4 | rs4441276 | TGTCTGCTTCAGGAAAAAACAGTTTCCTATNCCTCTTTCTCATAGCTGGTACTCAGAAAAA |
| HS3ST4 | rs12921387 | CAGAAATCCAAAATATTTTGCTTTTTATTTNATAAATCATGCAATGTTCTAGAAAAATTAG |
| IGLC1 | rs1065464 | TGCTCTGGAGATGCATTGCCAAAAAAATATNCTTATTGGTACCAGCAGAAGTCAGGCCAGG |
| IGLC1 | rs4820495 | gtttgttcctacaaccagtgtggaaatgtgNctaatcagaggcatcaggttaactaatcag |
| IL12RB1 | rs273493 | atacaagggaagtccctcatagcataagccNtttggaggctgaggttccaggagtcaccat |
| ITGAX | rs2230429 | ATGCTGTTCTCTACGGGGAGCAGGGCCACCNCTGGGGTCGCTTTGGGGCGGCTCTGACAGT |
| ITGAX | rs11574630 | TCCAGGGTCCTCCTCTCCTGCCTCCTCCGCNAGAGGTGGACCTCAACCCGGGGAAAGGGGG |
| MASP1 | rs12638131 | GCAGGAACCCTGAGGCGTGGAGAAAGCAGANATGTCCAGGGTCACCCAGCAGGTTGGTTTC |
| MASP2 | rs12142107 | taggtggcgccatcggataagggcaaggctgNgctgcgcagaggaaaccaggcttgttggttt |
| MYOC | rs2236875 | CCCTCAGCCCTAGGTGCCTATGGAGTTCACNTCTATCTATAGTTGCTCTTTCATCACGgtt |
| MYOC | rs12035960 | AGCCTGCTGGCTTCTTCTAGGTCATGTCAGNCAGGAGCATCTGGCAATGGTCAGACTCCAG |
| MYOC | rs235868 | ctgcccctoccaggggtagccagttcctacNgttagcaaaggactcacctgggaggacagc |
| PPID | rs8396 | TGCAATAAGAAAATGTAAAGGTTTTTGTCTNTGAATATGATCCCTAATGTGTTTCTTTTGA |
| PPID | rs7689418 | TAGCTTTATACTTTTTTGTAGGCTCTTGAANTAGACCCATCAAATACCAAAGCATTGTACC |
| PTPRC | rs1932433 | TTTTTTTAACATAATTCCTGATCTTAATTTNGATTACTCTAAGCAAATTTTTTTATCAATA |
| PTPRC | rs17670373 | ACATATTGTTTAAATTGAATCTTTATGATANTGCTTTATACTTCTCATTGTTTGGTAAACT |
| SLC2A2 | rs7646014 | CTCATTAGTCAGATACAGACATTCAAAAGCNAAGCACATCTGAAAAATCTAGGACCATAAT |
| SLC2A2 | rs1604038 | caaaccaaatccaagagtatgtcaaaaagaNaattcattgtgatcaagtagattttatttc |
| SLC2A2 | rs5400 | CTGTATCCAGCTTTGCAGTTGGTGGAATGANTGCATCATTCTTTGGTGGGTGGCTTGGGGA |
| SLC2A2 | rs11721319 | tgggcaaaggacatgaacagacatgtcctcNaaagaagacatacaagtggccaacatacat |
| SPOCK | rs1229729 | ACATGGGAAGCATGTATGTTGATAAAATAGNAACTCATGTCCCTTGAAAACTGATCAGACT |
| SPOCK | rs1229731 | GGCAAATTTTTCTTCTGATCTTAGCACCCANGCATTCATAGATAGCTCACTCTCAACATGC |
| SPOCK | rs2961633 | GTCCAGAGCAGCAGGGGGTCTTGCTCCTCANTTGAAAAGACAATCTGCTTTGCTCACCCAG |
| SPOCK | rs2961632 | AGCATGAGTTTGGTGGGTCATTATGTGTTANGTAAGGAGAGAGTTTACTAACAGTGTAAAG |
| SPOCK | rs12656717 | AGCACTTTAAAAAAAACTGCTTATCTTGTCNtccattttgtgttgctatgaaagaatacct |
| TGFBR2 | rs4955212 | TGGGGACTTTAATGatgctccataactgccNtattcattatgatacccaagagccacctgt |
| TGFBR2 | rs1019855 | ACACAAGCTGACTGAGTAAAGTATATTTAGNTCATTTCCAAATGACCAGGCTTTAGACCAA |
| TGFBR2 | rs2082225 | GCTGCAATCCAAGGCAGCGATCATCACTATNGTTGTTCTATGAGTTCCCACAGCCTAGGCC |
| TGFBR2 | rs9823731 | GTTGGGTCTACCCATCAAGTAACATGTATTNTATCTCGCCAGGGGCCCCGTTACAGTGGTG |

TABLE VIII
Flanking Sequences for SNPs shown in Table II

| Gene | SNP | SNP Flanking Sequence |
|---|---|---|
| C3 | rs2547438 | tctttgcctctcctaagcctgtgcccctgctNcccctggggccccctctggctggcacctcaa |
| C3 | rs2230199 | aaggtggcctgcacggtcacgaacttgttgcNccccttttctgacttgaactccctgttggct |
| C3 | rs1047286 | caaagacttccccaccaggtcttctgctcggNggttctgcaccccgtccagcagtaccttccg |
| C3 | rs3745567 | tgagatagagagcagaaagcaaggatggggtNaccggtgtgtccacacatggcagtcatcccc |
| C3 | rs11569507 | CTGCCCAGACCCCCTGATTCTGAATCTGCANGGGGGGATGACTGCCATGTGTGGACACACC |
| C3 | rs11085197 | cctctcagaccggcccacttggtggtcctgaNcctggccttcagactgggcctcacctgagtg |
| C7 | rs2271708 | CTATGTTAGGAGGAGGTTTATCGATATCACNGGAAGGTCTCCTTTCTGAGTCCTCACATCT |
| C7 | rs1055021 | CTGATTTGACATGCACTGACCTCTCCTATANCCCTCACTGGAGAAGGGCACAGATCACAGA |
| C9 | rs476569 | TCTGTTAGCTCTGGGTCATAACTAAGATAANAGAACATCCCAGTTTATAATGACCATTGTG |
| C1NH | rs2511988 | AGCTGCCCCACCTAGAAAATAAGAGATGCANCTTAACAGTCTTCCTACCGCATCTCTCTCC |
| C1NH | rs4926 | GTCTTTGAAGTGCAGCAGCCCTTCCTCTTCNTGCTCTGGGACCAGCAGCACAAGTTCCCTG |
| ITGA4 | rs3770115 | ACAATTTTGATGATCCCTTATTTACAGGAANGATGCCAAGAAACACAGGACTAAATAAACC |
| ITGA4 | rs4667319 | AGTGCAATGCAGACCTTGAAAGGCATAGTCCNGTTCTTGTCCAAGACTGATAAGAGGCTATT |

1. A method of screening for susceptibility to complement dysregulation in an individual comprising screening for the presence or absence of a genetic profile characterized by polymorphisms in the genome of the individual associated with complement dysregulation, wherein the presence of a said genetic profile is indicative of the individual's risk of complement dysregulation, wherein the genetic profile comprises at least one polymorphism selected from Table I or Table II.

2. A method for determining an individual's risk for development or progression of age-related macular degeneration (AMD) comprising screening for the presence or absence of a risk profile characterized by polymorphisms in the genome of the patient associated with risk for developing or with protection against developing AMD in genomic regions selected from at least one gene selected from Table I or II, and combinations thereof, wherein the presence of a said risk profile indicates that the patient has or is at risk of developing AMD.

3. The method of claim 2 comprising screening at least two of said genes.

4. The method of claim 2 comprising screening at least five of said genes.

5. The method of claim 2 comprising screening for at least ten of said genes.

6. The method of claim 2 comprising screening for a combination of at least one risk-associated polymorphism and at least one protective polymorphism.

7. The method of claim 2 comprising screening for a SNP selected from the group consisting of: rs7380703, rs10057855, rs1676717, rs1932433, rs1065464, rs4441276, rs947367, rs8396, rs1229729, rs1229731, rs10057405, rs17670373, rs331075, rs12714097, rs7646014, rs7338606, rs3770115, rs1874573, rs2236875, rs12992087, rs1621212, rs7689418, rs4926, rs11842143, rs12035960, rs12189024, rs2961633, rs2981582, MRD_{—}4048, rs17676236, rs6891153, MRD_{—}4044, rs10117466, rs2826552, rs2271708, rs1055021, and rs1055021, or combinations thereof.

8. The method of claim 2 comprising screening additionally for genomic deletions associated with AMD risk or AMD protection.

9. The method of claim 2 comprising screening for one or more additional AMD risk-associated or protection-associated polymorphisms in the genome of said patient.

10. The method of claim 9 comprising screening for an additional polymorphism selected from the group consisting of: a polymorphism in exon 22 of CFH (R1210C), rs2511989, rs1061170, rs203674, rs1061147, rs2274700, rs12097550, rs203674, rs9427661, rs9427662, rs10490924, rs11200638, rs2230199, rs800292, rs3766404, rs529825, rs641153, rs4151667, rs547154, rs9332739, rs3753395, rs1410996, rs393955, rs403846, rs1329421, rs10801554, rs12144939, rs12124794, rs2284664, rs16840422, and rs6695321.

11. The method of claim 9, comprising screening for an additional polymorphism selected from Table VI.

12. The method of claim 2 wherein the screening step is conducted by inspecting a data set indicative of genetic characteristics previously derived from analysis of the patient's genome.

13. The method of claim 2 wherein the screening comprises analyzing a sample of said patient's DNA or RNA.

14. The method of claim 2 wherein the screening comprises analyzing a sample of said individual's proteome to detect an isoform encoded by an allelic variant in a protein thereof consequent of the presence of a said polymorphism in said individual's genome.

15. The method of claim 2 wherein the screening comprises combining a nucleic acid sample from the subject with one or more polynucleotide probes capable of hybridizing selectively to DNA or RNA comprising a said polymorphism in a said genomic region.

16. The method of claim 2 wherein the screening comprises sequencing selected portions of the genome or transcriptome of said patient.

17. The method of claim 2 wherein said patient is determined to be at risk of developing AMD symptoms comprising the additional step of prophylactically or therapeutically treating said patient to inhibit development thereof.

18. The method of claim 2 comprising the further step of producing a report identifying the patient and the identity of the alleles at the sites of said one or more polymorphisms.

19. A detectably labeled oligonucleotide probe for hybridization with a genomic region of said patient for identification of the base present in said region at said polymorphism, wherein the genomic region is selected from at least one gene selected from Table I or II.

20. (canceled)

21. A method for treating or slowing the onset of AMD, the method comprising prophylactically or therapeutically treating a patient identified as having a risk profile characterized by polymorphisms in the genome of the patient associated with risk for developing or in genomic regions selected from at least one gene selected from Table I or II, and combinations thereof, wherein the presence of a said risk profile indicates that the patient has or is at risk of developing AMD.

22. (canceled)
